(19) United States
(12) Patent Application Publication
(10) Pub. No.: US 2024/0188561 A1
(43) Pub. Date: Jun. 13, 2024

(54) ADDITIVES FOR ENHANCING THE PESTICIDAL EFFECTIVENESS OF PESTICIDAL MICROORGANISMS

(71) Applicant: BASF SE, Ludwigshafen am Rhein (DE)

(72) Inventors: Timothy H. ANDERSON, Cincinnati, OH (US); Dean A. OESTER, Cincinnati, OH (US); David J. RODRIGUEZ, Tarrytown, NY (US)

(21) Appl. No.: 18/553,452

(22) PCT Filed: May 2, 2022
(86) PCT No.: PCT/EP22/61649

§ 371 Date: Sep. 29, 2023

(30) Foreign Application Priority Data
May 3, 2021 (EP) 21171741.8

(51) Int. Cl. A01N 25/30; A01N 63/22; A01N 63/27; A01P 1/00; A01P 3/00

(52) U.S. Cl. CPC A01N 25/30; A01N 63/22; A01N 63/27; A01P 1/00; A01P 3/00

(57) ABSTRACT

Disclosed herein are additives and additive blends, capable of enhancing the pesticidal effectiveness of pesticidal microorganisms, spraying liquids including such additives and additive blends and methods to protect plants or plant parts from fungal or bacterial attack using such spraying liquids.

### FIELD OF THE INVENTION

The present disclosure relates to additives and additive blends, capable to enhance the pesticidal effectiveness of pesticidal microorganisms.

### BACKGROUND OF THE INVENTION

Pesticidal microorganisms, also referred to as “microbials” or “biologicals” play an increasingly important role for protecting crops against various pests. Such pesticidal microorganisms are often applied in water based spraying liquids, prepared from wettable powder or aqueous or non-aqueous suspension formulations. The exact make-up of these formulations is usually carefully selected to ensure a) stability of the formulation during storage, b) a high survival rate of the pesticidal microorganism during storage, c) a low phytotoxicity after application and d) safety and ease of use during preparation of the spraying liquids and their application. Exemplary formulation types for pesticidal microorganisms are described in WO2018/067815, WO2009/126473, WO2020/205912, WO2015/184170, WO2016/109332, WO2010/128003, WO2009/037242, WO2018/128986 and WO2018/128985. Pesticidal microorganism are more dependent on environmental conditions for reliable full effectiveness than synthetic pesticides. Accordingly, there is a need to identify technical solutions to enhance the reliability and effectiveness of pesticidal microorganisms. However, the need to reach the technical requirement to ensure a high survival rate of the formulated microorganism during storage and transport limit the possibility to further enhance the pesticidal effectiveness of the formulated microorganism. Based on this situation, it was one objective of the present invention to identify additives and additive mixtures, which improve the pesticidal effectiveness of pesticidal microorganisms. It was a further objective of the invention to provide additives and additive mixtures which enhance the pesticidal effectiveness of pesticidal microorganisms and have low phytotoxic effects, having low negative or even supporting effects for survival and/or growth of the pesticidal microorganism on the plant, soil or plant propagation material after application. These objectives have been achieved by the following means.

### SUMMARY OF THE CLAIMED INVENTION

The invention comprises spraying liquids suitable for spraying plants, comprising:
a. 0.1%-v/v to 2%-v/v of at least one additive selected from the groups i. to x.:
   i. EO-PO block polymers having a HLB (hydrophilic-lipophilic-balance) value of 1, 2, 3, 4, 6, 8, 12, 13, 14, 15, 27 or 29 and an average molecular weight (AMW) between 1500 and 15000,
   ii. methyl esters of unsaturated or saturated C6 to C18 fatty acids,
   iii. ethylenediamine tetra-functional PO/EO block polymers with a molecular weight (AMW) between 2000 and 20000,
   iv. C8 to C14 alkyl polyglycosides,
   v. fatty acid polyethylene esters comprising C12 to C18 saturated or unsaturated fatty acids
   vi. ethoxylated triglycerides derived from plant oils,
   vii. alkoxylated alcohols, having from 2 to 80 oxyethylene units and from 2 to 40 oxypropylene units and C4 to C18 alcohols,
   viii. alkyl naphthalene sulfonates,
   ix. alkyl polyglycoside blended lignosulfonates,
   x. polyquaternium polymers,
b. 0.002%-v/v to 2.0%-v/v bacterial spores or vegetative cells,
c. optionally 0.001%-v/v to 10.0%-v/v of further components, and
d. up to 100%-v/v water.

In some embodiments the spraying liquids comprise a blend of additives comprising one or more additives selected from the group of ethoxylated triglycerides, preferably selected from ethoxylated soybean oil with POE 10, 30, 42 or 60, ethoxylated castor oil with POE 2.5, 5, 7, 16, 18, 20, 25, 30, 33, 35, 36, 40, 44, 60 or 200, ethoxylated rapeseed oil with POE 30 and one or more additives selected from the groups of a) to d):
a) EO-PO block polymers having a HLB (hydrophilic-lipophilic-balance) value of 1, 2, 3, 4, 6, 8, 12, 13, 14, 15, 27 or 29 and an average molecular weight between 1500 and 15000,
b) fatty acid polyethylene esters,
c) alkoxylated alcohols,
d) alkyl naphthalene sulfonates,
   wherein the % v/v ratio of the additives of the two groups is between 10:1 and 1:1, and if one or more additives of the group of ethoxylated triglycerides and/or group a), b), c) or d) is present, the total amount of all additives per group is calculated to determine the ratio.

Some spraying liquid comprise a blend of additives as defined in a. to g:
a. an additive blend comprising 1 to 99% v/v of at least one additive selected from: soybean oil POE 10, castor oil POE 16 and castor oil POE 40, preferably at least one or all is soybean oil POE 10, and 1 to 99% v/v of at least one additive selected from: PEG 400 monolaurate, PEG 400 monooleate and PEG 400 dioleate, preferably at least one or all is PEG 400 dioleate, wherein the % v/v of both components add up to 100%;
b. an additive blend comprising 1 to 98% v/v of at least one additive selected from: soybean oil POE 10, castor oil POE 16 and castor oil POE 40, preferably at least one or all is soybean oil POE 10, and 1 to 98 v/v of at least one additive selected from: PEG 400 monolaurate, PEG 400 monooleate and PEG 400 dioleate, preferably at least one or all is PEG 400 dioleate and 1 to 98% v/v of at least one additive selected from: alkoxylated alcohol having an HLB value of 9 or 12, preferably at least one or all has an HLB value of 12, wherein the % v/v of all components add up to 100%;
c. an additive blend comprising 1 to 99% v/v of at least one additive selected from: soybean oil POE 10, castor oil POE 16 and castor oil POE 40, preferably at least one or all is soybean oil POE 10, and 1 to 99% v/v of dibutyl naphthalene sulfonate, preferably sodium dibutyl naphthalene sulfonate, wherein the % v/v of all components add up to 100%;
d. an additive blend comprising 1 to 98% v/v of at least one additive selected from: soybean oil POE 10, castor oil POE 16 and castor oil POE 40, preferably at least one or all is soybean oil POE 10, and 1 to 98% v/v of at least one additive selected from: PEG 400 monolaurate, PEG 400 monooleate and PEG 400 dioleate, preferably at least one or all is PEG 400 dioleate, and 1 to 98% v/v of at least one additive selected from: EO/PO/EO block polymers with an HLB value of 1, 2, 3, 12, 13, 14, 15, 27 and 29, preferably at least one or all have an HLB value of 2 or 12, more preferred at least one or all have a HLB value of 2, wherein the % v/v of all components add up to 100%;
e. an additive blend comprising 1 to 95% v/v of at least one additive selected from: soybean oil POE 10, castor oil POE 16 and castor oil POE 40, preferably at least one or all is soybean oil POE 10, and 1 to 95% v/v of at least one additive selected from: PEG 400 monolaurate, PEG 400 monooleate and PEG 400 dioleate, preferably at least one or all is PEG 400 dioleate, and 1 to 95% v/v dibutyl naphthalene sulfonate, preferably sodium dibutyl naphthalene sulfonate, and 1 to 95% v/v of at least one additive selected from: alkoxylated alcohol having an HLB value of 9 or 12, preferably at least one or all has an HLB value of 12, and 1 to % v/v at least one selected from: EO/PO/EO block polymers with an HLB value of 1, 2, 3, 12, 13, 14, 15, 27 and 29, preferably at least one has an HLB value of 2, 12, or 15, more preferred at least one has an HLB value of 2, wherein the % v/v of all components add up to 100%;
f. an additive blend comprising 1 to 97% v/v of at least one additive selected from: soybean oil POE 10, castor oil POE 16 and castor oil POE 40, preferably at least one or all is soybean oil POE 10, and 1 to 97% v/v dibutyl naphthalene sulfonate, preferably sodium dibutyl naphthalene sulfonate, and 1 to 97% v/v an alkoxylated alcohol having an HLB value of 9 or 12, preferably at least one or all has an HLB value of 12, wherein the % v/v of all components add up to 100%;
g. an additive blend comprising 1 to 97% v/v of at least one additive selected from: EO/PO/EO block polymers with an HLB value of 1, 2, 3, 12, 13, 14, 15, 27 and 29, preferably at least one or all have an HLB value of 2, or 12, more preferred at least one or all have a HLB value of 2, and 1 to 97% v/v of at least one additive selected from: PEG 400 monolaurate, PEG 400 monooleate and PEG 400 dioleate, preferably at least one or all is PEG 400 dioleate, and 1 to 97% v/v of at least one additive selected from: soybean oil POE 10, castor oil POE 16 and castor oil POE 40, preferably at least one or all is soybean oil POE 10, wherein the % v/v of all components add up to 100%.

Preferably the spraying liquid comprise bacterial spores or vegetative cells from the genus Bacillus, Lysinibacilllus, Paenibacillus, Streptomyces Pseudomonas, Burkholderia, Paraburkholderia or Rhizobium.

Preferred additive blends to prepare the spraying liquids of the invention are additive blends comprising one or more additives selected from the group of ethoxylated triglycerides, preferably selected from ethoxylated soybean oil with POE 10, 30, 42 or 60, ethoxylated castor oil with POE 2.5, 5, 7, 16, 18, 20, 25, 30, 33, 35, 36, 40, 44, 60 or 200, ethoxylated rapeseed oil with POE 30 and one or more additives selected from the group A1) to D1):
A1) EO-PO block polymers having a HLB (hydrophilic-lipophilic-balance) value of 1, 2, 3, 4, 6, 8, 12, 13, 14, 15, 27 or 29 and an average molecular weight between 1500 and 15000,
B1) fatty acid polyethylene esters,
C1) alkoxylated alcohols,
D1) alkyl naphthalene sulfonates,
wherein the % v/v ratio of the additives of the two groups is between 10:1 and 1:1, and if one or more additives of the group of ethoxylated triglycerides and/or group A1), B1), C1) or D1) is present, the total amount of all additives per group is calculated to determine the ratio.

Other preferred additive blends comprise one or more additives selected from the group of EO-PO block polymers having a HLB (hydrophilic-lipophilic-balance) value of 1, 2, 3, 4, 6, 8, 12, 13, 14, 15, 27 or 29 and an average molecular weight between 1500 and 15000 and one or more additives selected from thegroup of A2) to C2):
A2) fatty acid methyl esters preferably C6-C10 methyl caproate-caprylate-caprate, C8-C10 methyl caprylate-caprate, C8 methyl caprylate, C10 methyl caprate, C12 methyl laurate, methyl coconate, palm kernel methyl ester, C14 methyl myristate, C16 methyl palmitate, C18 methyl stearate, methyl sunflowerate, palm oil methyl ester, methyl rapeate, methyl soyate and C18:1 methyl oleate more preferred C6-C10 fatty acid methyl ester, C12-C18 fatty acid methyl ester, soya fatty acid methyl ester, oleic acid methyl ester. More preferred are C6-C10 fatty acid methyl ester, soya fatty acid methyl ester, preferably a methyl soyate, and oleic acid methyl ester, preferably a C18:1 methyl oleate;
B2) ethylenediamine tetra-functional PO/EO block polymers, preferably ethylenediamine tetrafunctional PO/EO block polymers with a molecular weight (AMW) between 2000 and 20000, more preferred between 3000 and 18000, even more preferred they have a molecular weight of 3600, 4700 or 15000 and preferably have an HLB of 3 or 24,
C2) fatty acid polyethylene esters, preferably PEG 400 monolaurate, PEG 400 monooleate, PEG 400 dioleate more preferred is PEG 400 dioleate.
   wherein the % v/v ratio of the additives of the group of EO-PO block polymers and the one or more additives of the groups of A2), B2) or C2) is between 10:1 and 1:1, and if one or more additives of the group of EO-PO block polymers and/or group A2), B3), or C2) is present, the total amount of all additives per group is calculated to determine the ratio.

Alternative additive blends comprise one or more additives selected from the group of EO/PO/EO block polymers having a HLB (hydrophilic-lipophilic-balance) value of 1, 2, 3, 4, 6, 8, 12, 13, 14, 15, 27 or 29 and an average molecular weight between 1500 and 15000 and one or more additives selected from the group of PO/EO/PO block polymers with an HLB value of 4, 6, 8, or 12 and an average molecular weight between 1500 and 4000,
wherein the % v/v ratio of the EO/PO/EO block polymers and the PO/EO/PO block polymers is between 100:1 and 1:1, and if one or more additives of the group of EO/PO/EO block polymers and/or the group of PO/EO/PO block polymers is present, the total amount of all additives per group is calculated to determine the ratio.

Further preferred additive blends comprise a blend of additives as defined in any one of the alternatives a. to g.:
a. comprising 1 to 99% v/v of at least one additive selected from: soybean oil POE 10, castor oil POE 16 and castor oil POE 40, preferably at least one or all is soybean oil POE 10, and 1 to 99% v/v of at least one additive selected from: PEG 400 monolaurate, PEG 400 monooleate and PEG 400 dioleate, preferably at least one or all is PEG 400 dioleate,
b. comprising 1 to 98% v/v of at least one additive selected from: soybean oil POE 10, castor oil POE 16 and castor oil POE 40, preferably at least one or all is soybean oil POE 10, and 1 to 98 v/v of at least one additive selected from: PEG 400 monolaurate, PEG 400 monooleate and PEG 400 dioleate, preferably at least one or all is PEG 400 dioleate and 1 to 98% v/v of at least one additive selected from: alkoxylated alcohol having an HLB value of 9 or 12, preferably at least one or all has an HLB value of 12,
c. comprising 1 to 99% v/v of at least one additive selected from: soybean oil POE 10, castor oil POE 16 and castor oil POE 40, preferably at least one or all is soybean oil POE 10, and 1 to 99% v/v of dibutyl naphthalene sulfonate, preferably sodium dibutyl naphthalene sulfonate,
d. comprising 1 to 98% v/v of at least one additive selected from: soybean oil POE 10, castor oil POE 16 and castor oil POE 40, preferably at least one or all is soybean oil POE 10, and 1 to 98% v/v of at least one additive selected from: PEG 400 monolaurate, PEG 400 monooleate and PEG 400 dioleate, preferably at least one or all is PEG 400 dioleate, and 1 to 98% v/v of at least one additive selected from: EO/PO/EO block polymers with an HLB value of 1, 2, 3, 12, 13, 14, 15, 27 and 29, preferably at least one or all have an HLB value of 2 or 12, more preferred at least one or all have a HLB value of 2,
e. comprising 1 to 95% v/v of at least one additive selected from: soybean oil POE 10, castor oil POE 16 and castor oil POE 40, preferably at least one or all is soybean oil POE 10, and 1 to 95% v/v of at least one additive selected from: PEG 400 monolaurate, PEG 400 monooleate and PEG 400 dioleate, preferably at least one or all is PEG 400 dioleate, and 1 to 95% v/v dibutyl naphthalene sulfonate, preferably sodium dibutyl naphthalene sulfonate, and 1 to 95% v/v of at least one additive selected from: alkoxylated alcohol having an HLB value of 9 or 12, preferably at least one or all has an HLB value of 12, and 1 to % v/v at least one selected from: EO/PO/EO block polymers with an HLB value of 1, 2, 3, 12, 13, 14, 15, 27 and 29, preferably at least one has an HLB value of 2, 12, or 15, more preferred at least one has an HLB value of 2,
f. comprising 1 to 97% v/v of at least one additive selected from: soybean oil POE 10, castor oil POE 16 and castor oil POE 40, preferably at least one or all is soybean oil POE 10, and 1 to 97% v/v dibutyl naphthalene sulfonate, preferably sodium dibutyl naphthalene sulfonate, and 1 to 97% v/v an alkoxylated alcohol having an HLB value of 9 or 12, preferably at least one or all has an HLB value of 12,
g. comprising 1 to 97% v/v of at least one additive selected from: EO/PO/EO block polymers with an HLB value of 1, 2, 3, 12, 13, 14, 15, 27 and 29, preferably at least one or all have an HLB value of 2, or 12, more preferred at least one or all have a HLB value of 2, and 1 to 97% v/v of at least one additive selected from: PEG 400 monolaurate, PEG 400 monooleate and PEG 400 dioleate, preferably at least one or all is PEG 400 dioleate, and 1 to 97% v/v of at least one additive selected from: soybean oil POE 10, castor oil POE 16 and castor oil POE 40, preferably at least one or all is soybean oil POE 10,
   wherein the % v/v of all components of additive blends a. to g. and optionally water add up to 100%.

Another embodiment of the invention is a kit of at least two parts to prepare a spraying liquid suitable for spraying plants or for preparing a seed treatment composition for treating seeds of plants, wherein the bacterial spores or vegetative cells are provided in a first concentrated form and at least one of the additive blends is provided in at least one further concentrated form and wherein the relative amounts of the bacterial spores or vegetative cells and the relative amounts of the additive in the kit are adapted to provide the amounts of the final spraying liquid.

The invention further comprises a method to control phytopathogenic fungi or phytopathogenic bacteria wherein an additive blend or a kit of parts is used to prepare a spraying liquid for spraying plants or for preparing a seed treatment composition for treating seeds and the spraying liquid for spraying plants is sprayed on plants or the plant seeds are treated with the seed treatment composition for treating seeds.

Further encompassed by the invention are the use of an additive blend to enhance the pesticidal effectiveness of a pesticidal microorganism in a method to control phytopathogenic fungi or phytopathogenic bacteria, the use of an additive blend to prepare a spraying liquid to enhance the pesticidal effectiveness of the comprised pesticidal microorganisms, the use of a spraying liquid comprising additives to enhance the pesticidal effectiveness of the comprised pesticidal microorganisms in a method to control phytopathogenic fungi or phytopathogenic bacteria and the use of a kit of at least two parts to prepare such spraying liquids.

### DETAILED DESCRIPTION

The spraying liquids of the invention comprise one or more additives and pesticidal microorganisms in the form of spores or cells and are capable to enhance the pesticidal effectiveness of such pesticidal microorganism in methods to protect plants or plant material from attack of phytopathogenic fungi and/or bacteria. The enhanced pesticidal effectiveness of such pesticidal microorganism results in a higher protection of the plants or plant material against fungal and/or bacterial attack in comparison to a situation in which the same amount of pesticidal microorganism is applied without the additives or additive blends to plants or plant parts and such plants are under attack by the same fungus and/or bacterial species.

“Spores” as contemplated by the present invention refers to viable reproductive units in the form of endo- or exospores of a bacterial species belonging to the phylum Firmicutes or Actinobacteria. It is further recognized that the spores disclosed herein are produced via culturing of the bacteria and are usually harvested from the fermentation broth before used to prepare pesticidal formulations or the spraying liquids of the invention. They can therefore comprise a combination of vegetative cells and forespores (cells in an intermediate stage of spore formation); a combination of forespores and spores; or a combination of forespores, vegetative cells and/or spores. “vegetative cells” refers to cells of bacterial species which are not in a dormant state and not in the process to produce spores. Vegetative cells are actively growing as soon as enough nutrients are available. While spores are usually preferred to produce formulations of pesticidal microorganism, viable cells are also used, in particular for bacterial species, which do not produce endo- or exospores, like species of the phylum Pseudomonadales.

For the purpose of the invention, the spores and vegetative cells have usually been harvested from a fermentation broth used to grow the respective pesticidal microorganism before they are used to produce pesticidal formulations. The process of spore or cell harvesting is performed via concentration and washing of the spores or vegetative cells via processes used in the art for these purposes, like centrifugation or filtration. These processes do usually not completely purify the harvested spores or vegetative cells from other solid components of the fermentation broth, so that the harvested spores or vegetative cells comprise also solid components of the fermentation broth to a certain degree, which is dependent on the exact type of the fermentation broth used and the method to harvest them. After harvest, the spores or vegetative cells are usually provided in form of a mush or pulp or, which is then preferably dried, e.g. via after freeze or spray drying, to form a dry powder to facilitate formulation. However, in some cases, the spraying liquids or formulations of pesticidal microorganisms used to prepare the spraying liquids, may be produced from liquid concentrates of spores or vegetative cells which still comprise liquid parts of the fermentation both.

The amount of spores or vegetative cells of the harvested material is usually determined in colony forming units (CFU) per gram of harvested material. Typically, the harvested material used for formulation comprises between 1×10⁸ CFU/g to 1×10¹¹ CFU/g.

The amount of spores and vegetative cells in the spraying liquids is preferably between 0.002%-v/v to 2.0%-v/v.

For spores of the phylum Firmicutes or Actinobacteria, e.g. for spores of Bacillus species the amount is preferably between 0.1%-v/v and 1.8%-v/v, or 0.2%-v/v and 1.6%-v/v, or 0.38%-v/v and 1.15%-v/v, or 0.5%-v/v and 1.55%-v/v.

For vegetative cells of the phylum Pseudomonadales, e.g. vegetative cells of Pseudomonas species, the amount is preferably between. 0.001%-v/v and 0.02%-v/v, or 0.003%-v/v and 0.015%-or 0.005%-v/v and 0.01%-v/v.

The total amount of additives in the spraying solutions is between 0.1%- to 2%-v/v, 0.2% to 2%-v/v, 0.3% to 2%-v/v, 0.4% to 2%-v/v, 0.5% to 2%-v/v, 0.6% to 2%-v/v, 0.7% to 2%-v/v, 0.8% to 2%-v/v, 0.9% to 2%-v/v, 0.1%- to 1.5%-v/v, 0.2% to 1.5%-v/v, 0.3% to 1.5%-v/v, 0.4% to 1.5%-v/v, 0.5% to 1.5%-v/v, 0.6% to 1.5%-v/v, 0.7% to 1.5%-v/v, 0.8% to 1.5%-v/v or 0.9% to 1.5%-v/v, 0.1%- to 1%-v/v, 0.2% to 1%-v/v, 0.3% to 1%-v/v, 0.4% to 1%-v/v, 0.5% to 1%-v/v, 0.6% to 1%-v/v, 0.7% to 1%-v/v, 0.8% to 1%-v/v or 0.9% to 1%-v/v. Conventional spraying liquids for pesticidal microorganisms are usually prepared by diluting agrochemical formulations comprising the microorganisms with water. Agrochemical formulations of pesticidal microorganism may also comprise additives, however, the kind and amount of these additives, as selected to create stable agrochemical formulations, are, due to the dilution effect, only present in a low amounts in conventional spraying solutions, usually below 500 ppm or 0.05%-v/v.

The spraying solutions of the inventions are usually prepared by diluting a concentrate comprising a pesticidal microorganism, usually an agrochemical formulation comprising a pesticidal microorganism, with water and adding an at least one additive, preferably an additive mixture, to an amount of 0.1%- to 2%-v/v of the final volume of the spraying solution. The final volume is the volume selected by the user for application on the plants or crops.

The additives used in the spraying liquids and in the additive mixtures are selected from groups i. to x.

Group i. consists of EO-PO block polymers. EO-PO block polymers are polyethylene glycol—polypropylene glycol block copolymers of the A-B-A type or the B-A-B type. in which A is a hydrophilic block and B is a hydrophobic block. EO-PO block polymers of the A-B-A type are also called poloxamers or EO/PO/EO block polymers. EO-PO block polymers of the B-A-B type are also called meroxapoles or PO/EO/PO block polymers.

As described above, poloxamers are nonionic triblock copolymers and are composed of a central hydrophobic chain of polyoxypropylene (poly(propylene oxide)) flanked by two hydrophilic chains of polyoxyethylene (poly(ethylene oxide)). Because the lengths of the polymer blocks can be customized, many different poloxamers exist that have different properties. For the generic term poloxamer, these copolymers are commonly named with the letter P (for poloxamer) followed by three digits: the first two digits multiplied by 100 give the approximate molecular mass of the polyoxypropylene core, and the last digit multiplied by 10 gives the percentage polyoxyethylene content (e.g. P407=poloxamer with a polyoxypropylene molecular mass of 4000 g/mo} and a 70% polyoxyethylene content). For the Pluronic® and Synperonic® tradenames, coding of these copolymers starts with a letter to define its physical form at room temperature (L=liquid, P=paste, F=flake (solid)) followed by two or three digits, The first digit (two digits in a three-digit number) in the numerical designation, multiplied by 300, indicates the approximate molecular weight of the hydrophobe; and the last digit×10 gives the percentage polyoxyethylene content (e.g., L61 indicates a polyoxypropylene molecular mass of 1800 g/mol and a 10% polyoxyethylene content).

Preferred EO-PO block polymers comprise polyethylene glycol-polypropylene glycol polyethylene glycol block polymers with a molar mass of approximately 950 to 6500 g/mol and a polyethylene oxide content of 10% to 50% w/w; preferably: poloxamers of the types polyethylene—polypropylene glycol block polymer with 10% ethyleneoxide and a molar mass of approximately 850 g/mol, polyethylene—polypropylene glycol block polymer with 40% ethylene oxide and a molar mass of approximately 3250 g/mol,

Preferred EO/PO/EO and PO/EO/PO block polymers have a HLB (hydrophilic-lipophilic-balance) value of 1, 2, 3, 4, 6, 8, 12, 13, 14, 15, 27 or 29 value, more preferred EO/PO/EO block polymers have a HLB value of 1, 2, 3, 12, 13, 14, 15, 27 or 29, even more preferred have a HLB value of 2. More preferred PO/EO/PO block polymers have a HLB value of 4, 6, 8, 12 or 15, and preferably have a HLB value of 4.

Preferred EO/PO/EO and PO/EO/PO block polymers have an average molecular weight (AMW) in Daltons between 1500 and 15000, preferably between 1800 and 15000, like 1850, 2000, 2750, 2900, 3800, 4200, 5900, 8400, or 14600. More preferred they have an average molecular weight of 2750. Preferred PO/EO/PO block polymers have an average molecular weight between 1500 and 4000, preferably between 1900 and 4000, like 1950, 2150, 2650, 3100, or 3600. More preferred they have an average molecular weight of 3100.

The average molecular weight (AMW) for EO/PO/EO and PO/EO/PO block polymers is calculated using the midpoint of the hydroxyl number specification of the individual EO/PO/EO or PO/EO/PO block polymer and the following equation 56100×[2/midpoint of the hydroxyl number]. For example, an EO/PO/EO block polymer having a hydroxyl number specification between 18 and 20, the average molecular weight is calculated as 56100 Daltons×[2/19]=5900 Daltons.

Preferred EO/PO/EO in the spraying liquids and additive blends have
an AMW of 1850 Daltons and an HLB value of 12, or
an AMW of 2000 Daltons and an HLB value of 3, or
an AMW of 2900 Daltons and an HLB value of 15, or
an AMW of 8400 Daltons and an HLB value of 29, or
an AMW of 2750 Daltons and an HLB value of 2, or
an AMW of 4200 Daltons and an HLB value of 14, or
an AMW of 3800 Daltons and an HLB value of 1, or
an AMW of 5900 Daltons and an HLB value of 13, or
an AMW of 14600 Daltons and an HLB value of 27.
more preferred EO/PO/EO block polymers have an AMW of 2750 Daltons and an HLB value of 2.

Preferred PO/EO/PO block polymers in the spraying liquids and additive blends have an AMW of 1950 Daltons and an HLB value of 15, or
an AMW of 2150 Daltons and an HLB value of 6, or
an AMW of 2650 Daltons and an HLB value of 12, or
an AMW of 3100 Daltons and an HLB value of 4, or
an AMW of 3600 Daltons and an HLB value of 8, or
more preferred PO/EO/PO block polymers have an AMW of 3100 Daltons and a HLB value of 4

Group ii. consists of fatty acid methyl esters. Fatty acid methyl esters are methyl esters of unsaturated or saturated fatty acids. Non-limiting examples of fatty acid methyl esters are: C6-C10 methyl caproate-caprylate-caprate, C8-C10 methyl caprylate-caprate, C8 methyl caprylate, C10 methyl caprate, C12 methyl laurate, methyl coconate, palm kernel methyl ester, C14 methyl myristate, C16 methyl palmitate, C18 methyl stearate, methyl sunflowerate, palm oil methyl ester, methyl rapeate, methyl soyate and C18:1 methyl oleate.

Preferred methyl esters are C6-C10 fatty acid methyl ester, C12-C18 fatty acid methyl ester, soya fatty acid methyl ester, oleic acid methyl ester. More preferred are C6-C10 fatty acid methyl ester, soya fatty acid methyl ester, preferably a methyl soyate, and oleic acid methyl ester, preferably a C18:1 methyl oleate.

Group iii. consists of ethylenediamine tetra-functional PO/EO block polymers. Ethylenediamine tetra-functional PO/EO block polymers are synthesized by the sequential reaction of the acceptor ethylenediamine molecule first with propylene oxide (PO) and then with ethylene oxide (EO) precursors, resulting in a four-arm PEO-terminated molecular structure, in which an ethylenediamine central group is bonded to four chains of PPO-PEO blocks. Preferred are ethylenediamine tetra-functional PO/EO block polymers with a molecular weight (AMW) in Daltons between 2000 and 20000. More preferred between 3000 and 18000. In one embodiment they have a molecular weight of 3600, 4700 or 15000. Preferably they have an HLB of 3 or 24.

The average molecular weight (AMW) for ethylenediamine tetra-functional PO/EO block polymers is calculated using the midpoint of the hydroxyl number specification of the individual ethylenediamine tetra-functional PO/EO block polymers and the following equation 56100×[2/midpoint of the hydroxyl number].

Preferred ethylenediamine tetra-functional PO/EO block polymers in the spraying liquids and additive blends have an
an AMW of 3600 Daltons and an HLB value of 3, or
an AMW of 4700 Daltons and an HLB value of 3, or
an AMW of 15000 Daltons and an HLB value of 24.

Group iv. consists of alkyl polyglycosides. Alkyl polyglycosides are non-ionic and comprise sugars, usually glucose derivatives, reacted with fatty alcohols. In many cases they are produced from complex sources like starch and fat and consist therefore of complex mixtures of compounds with different sugars and alkyl groups of variable length. Non-limiting examples for suitable alkyl polyglycosides are C8-C10 alkyl polyglycosides, C8-C16 alkyl polyglycosides, C9-C11 alkyl polyglycosides, C12-C16 alkyl polyglycosides and C12-C16 alkyl polyglycosides, Preferred are alkyl polyglycosides with a specific degree of polymerization of the sugar moeties (D.P.), like C8-C10 alkyl polyglycosides with a D.P. of 1.5, C8-C10 alkyl polyglycosides with a D.P. of 1.7, C8-C16 alkyl polyglycosides with a D.P. of 1.6, C9-C11 alkyl polyglycosides with a D.P. of 16, C12-C16 alkyl polyglycosides with a D.P. of 1.4 and C12-C16 alkyl polyglycosides with a D.P. of 1.6.

More preferred are alkyl polyglycosides are C8-C10 alkylpolyglycosides and C12-C16 alkylpolyglycosides. Even more preferred are C8-C10 alkylpolyglycosides.

Group v. consists of fatty acid polyethylene esters. Fatty acid polyethylene (PEG) esters are non-ionic and synthesized by combining a fatty acid with polyethylene glycol. The fatty acid part usually comprises C12 to C18 saturated or unsaturated fatty acids.

Preferred fatty acid polyethylene ester additives are PEG 400 monolaurate, PEG 400 monooleate, PEG 400 dioleate more preferred is PEG 400 dioleate.

Group vi. consists of ethoxylated triglycerides. Ethoxylated triglycerides are produced by combining several moles of ethylene oxide with every mole of triglyceride or by alternate routes of hydrotalcite catalyst or triglyceride transesterification with glycerin. The triglycerides are usually derived from plant oils, for example soybean oil, castor oil or rapeseed oil. Non-limiting examples are: ethoxylated soybean oil with POE 10, 30, 42 or 60, ethoxylated castor oil with POE 2.5, 5, 7, 16, 18, 20, 25, 30, 33, 35, 36, 40, 44, 60 or 200, ethoxylated rapeseed oil with POE 30. Preferred ethoxylated triglyceride additives are soybean oil POE 10, soybean oil POE 30, castor oil POE 16, castor oil POE 40, or mixtures thereof. Industrial production of ethoxylated triglycerides results in a Poisson distribution of hydrophilic chain lengths, attached to a generally hydrophobic portion.

The POE number of an ethoxylated triglyceride is therefore a weighted average of all hydrophilic chain lengths present in the ethoxylated triglyceride, with the weighted average typically representing 75% of the hydrophilic chain lengths present.

Group vii. consists of alkoxylated alcohols. Alkoxylated alcohols are produced by combining ethylene oxide and/or propylene oxide with an alcohol. Preferred alkoxylated alcohols have from 2 to 80 oxyethylene units and from 2 to 40 oxypropylene units, wherein the alcohols are preferably C4 to C18 alcohols, more preferably C10 to C13 alcohols, most preferred C10 alcohols, wherein the oxyethylene units are preferably 3 to 40 oxyethylene units and 3 to 20 oxypropylene units more preferred from 7 to 16 oxyethylene units and 3 to 10 oxypropylene units. Preferred alkoxylated alcohol additives have an HLB (hydrophilic-lipophilic-balance) value of 2.8, 3.4, 8, 9, 10.6 or 12 More preferred have an HLB value of 9 or 12, even more preferred of 12. Also preferred is a POP 31 POE 31 butyl alcohol.

Group viii. consists of alkyl naphthalene sulfonates. Alkyl naphthalene sulfonates are derivatives of sulfonic acid which contain a at least one alkyl naphthalene functional unit. Preferred is dibutyl naphthalene sulfonate, more preferred the sodium salt of dibutyl naphthalene sulfonate.

Group ix. consists of alkyl polyglycoside lignosulfonate blends: Lignosulfonates are produced by sulfonation of lignin. The average molecular weight of the lignosulfonate is at least about 1,000 Da. For example, the average molecular weight of the lignosulfonate can be from about 1,000 Da to about 75,000 Da, from about 1,000 Da to about 50,000 Da, from about 1,000 Da to about 20,000 Da, with preferred molecular weights from about 1,000 Da to about 10,000 Da and the most preferred molecular weights from about 2,500 Da to about 4,000 Da.

Lignosulfonates can be characterized in part by the degree of sulfonation of the polymer molecule. For example, in some embodiments, the lignosulfonate has a degree of sulfonation that is from about 0.3 moles/kg to about 4 moles/kg, with preferred embodiments having a degree of sulfonation from about 0.5 moles/kg to about 4 moles/kg, and the most preferred degree of sulfonation from about 0.5 moles/kg to about 3.5 moles/kg. Lignosulfonates can also be characterized in part by content of organic sulfur. In various embodiments, the organic sulfur content of the lignosulfonate is from about 0.5 wt. % to about 20 wt. %, with a preferred sulfur content from about 2 wt. % to about 15 wt. %, and a most preferred sulfur content from about 4 wt. % to about 11 wt. %.

The sulfonic acid group of the lignosulfonate can be present at different locations on the polymer molecule. For example, the sulfonic acid group can be located on an aliphatic side chain and/or on an aromatic nucleus.

Lignosulfonates include various lignosulfonate salts such as sodium lignosulfonates, magnesium lignosulfonates, ammonium lignosulfonates, potassium lignosulfonates, calcium lignosulfonates, and combination thereof. In some embodiments, the polymeric additive comprises a sodium lignosulfonate.

Lignosulfonates can be spray dried with other surfactant chemistries to create a blended product which provide wider functionality. The lignosulfonates used in the invention are blended with alkylpolyglycosides. Non-limiting examples for suitable alkyl polyglycosides are C8-C10 alkyl polyglycosides, C8-C16 alkyl polyglycosides, C9-C11 alkyl polyglycosides, C12-C16 alkyl polyglycosides and C12-C16 alkyl polyglycosides,

A preferred blend is a lignosulfonate C8-C10 alkyl polyglycoside blend.

Group x. consists of polyquaternium polymers. Polyquaternium polymers comprise quaternary ammonium centers in the polymer. A preferred polyquaternium additive is polyquaternium-7.

In some embodiments the spraying liquids comprise spores of the phylum Firmicutes or Actinobacteria, preferably of the genus Bacillus, Lysinibacilllus or Paenibacillus or of the genus Streptomyces.

Preferred species of the genus Bacillus are Bacillus subtilis, Bacillus licheniformis, Bacillus firmus, Bacillus amyloliquefaciens, Bacillus velezenis, Bacillus mycoides, Bacillus simplex, Bacillus thuringiensis, and Bacillus pumilus. Preferably of the genus Bacillus subtilis and Bacillus amyloliquefaciens.

Preferred strains of the genus Bacillus are: B. amyloliquefaciens MBI600, B. amyloliquefaciens PTA-4838, B. amyloliquefaciens F727, B. amyloliquefaciens D747, B. amyloliquefaciens ENV503, B. amyloliquefaciens FZB24, B. amyloliquefaciens FZB42, B. amyloliquefaciens RTI301, B. licheniformis FMCH001, B. subtilis FMCH002, B. subtilis RTI477, B. subtilis DSM 24682, B. subtilis QST 713, B. subtilis IAB/BS03, B. subtilis BU1814, B. mycoides BMJ, B. firmus NCIM 2637, B. firmus I-1582, B. thuringiensis subsp. kurstaki strains EVB-113-19, ABTS 351, PB 54, SA 11, SA12 and EG 2348, B. thuringiensis subsp. aizawai strains ABTS-1857 and GC-91, B. thuringiensis subsp. israeliensis AM65-52, B. pumilus F33 (syn. INR7, AP18, F-22, BU 1433), B. simplex ABU-288, Bacillus sp. ITB105.

In some embodiments the spraying liquids comprise spores of B. amyloliquefaciens MBI600, B. subtilis QST 713, B. subtilis BU1814, B. firmus I-1582, B. pumilus F33 or B. simplex ABU-288. In some embodiments the spraying liquids comprise spores of B. amyloliquefaciens MBI600. B. amyloliquefaciens MBI600 has been renamed several times and is also known as B. subtilis MBI600 and B. velezensis MBI600.

A preferred species of the genus Lysinibacilllus is Lysinibacilllus sphaericus. Preferred strains of the genus Lysinibacilllus are Lysinibacillus sphaericus NRRL B-67351 and Lysinibacilllus sphaericus NRRL B-67486.

Preferred species of the genus Paenibacillus are Paenibacillus polymyxa, Paenibacillus peoriae, Paenibacillus kribbensis and Paenibacillus terrae.

Preferred strains of the genus Paenibacillus are: P. polymyxa PKB1, P. polymyxa JB05-01-1, P. polymyxa AC-1, P. polymyxa HY96-2, Paenibacillus sp. NRRL B-50972, NRRL B-67129, NRRL B-67304, NRRL B-67306 and NRRL B-67615, NRRL B-50374, NRRL B-67721, NRRL B-67723, NRRL B-67724, P. polymyxa VMC10/96, Paenibacillus sp. 10.6D, Paenibacillus sp. 9.4E, Paenibacillus sp. Lu16774, Lu17007 and Lu17015, P. polymyxa M1, P. polymyxa SC2 and P. polymyxa Sb3-1 and P. polymyxa E681.

Preferred species of the genus Streptomyces are Streptomyces sp. K61 and Streptomyces lydicus, for example Streptomyces lydicus WYEC 108.

Preferred spraying liquids comprise spores of the phylum Firmicutes or Actinobacteria, preferably of the genus Bacillus or Paenibacillus or of the genus Streptomyces and more preferred of the genus Bacillus and most preferred comprising spores of the strain B. amyloliquefaciens MBI600 comprise at least one additive selected from EO/PO/EO block polymers with an HLB value of 1, 2, 3, 12, 13, 14, 15, 27 or 29, PO/EO/PO block polymers with an HLB value of 4, 6, 8, 12 or 15, C6-C10 fatty acid methyl ester, C12-C18 fatty acid methyl ester, soya fatty acid methyl ester, oleic acid methyl ester, ethylenediamine tetra-functional PO/EO block polymers with an molecular weight of 3600, 4700 or 15000, C8-C10 alkylpolyglycosides, PEG 400 monolaurate, PEG 400 monooleate, PEG 400 dioleate, soybean oil POE 10, castor oil POE 16, castor oil POE 40, alkoxylated alcohol HLB 9 or 12, butyl alcohol POP 31 POE 31, dibutyl naphthalene sulfonate, or a lignosulfonate C8-C10 alkylpolyglycoside blend.

In some embodiments at least one additive is selected from the group comprising: EO/PO/EO block polymers with an HLB value of 2, 12, or 15, PO/EO/PO block polymer with an HLB value of 4, C6-C10 fatty acid methyl ester, C12-C18 fatty acid methyl ester, soya fatty acid methyl ester, oleic acid methyl ester, ethylenediamine tetra-functional PO/EO block polymer with a molecular weight of 15000, C8-C10 alkylpolyglycosides, PEG 400 dioleate, soybean oil POE 10, alkoxylated alcohol with an HLB value of 12, or a dibutyl naphthalene sulfonate.

In some embodiments at least one additive is selected from the group comprising: EO/PO/EO block polymers with an HLB value 2 or 12, C6-C10 fatty acid methyl ester, C12-C18 fatty acid methyl ester, soya fatty acid methyl ester, oleic acid methyl ester, PEG 400 dioleate, soybean oil POE 10, or dibutyl naphthalene sulfonate.

In some embodiments at least one additive is selected from the group comprising: EO/PO/EO block polymers with an HLB value of 2 or 12, C6-C10 fatty acid methyl ester, soya fatty acid methyl ester, PEG 400 dioleate, soybean oil POE 10, alkoxylated alcohol HLB 12, dibutyl naphthalene sulfonate.

In some embodiments at least one additive is selected from the group comprising: EO/PO/EO block polymers with an HLB value of 2 or 12, PEG 400 dioleate, soybean oil POE 10, alkoxylated alcohol with an HLB value of 12, or dibutyl naphthalene sulfonate,

In some embodiments the spraying liquid is prepared by adding one or more additives to the spraying tank comprising the spores or vegetative cells of one or more pesticidal microorganisms and water.

In case the spraying liquid comprises more than one additive, preferably the additives are provided as an additive blend, which comprises the additives in ratios, which are selected to achieve the ratios of the different additives to each other present in the final spraying liquids. These additive blends may comprise any combination of additives and ratios of the different additives disclosed herein as being comprised by the spraying liquids.

In some embodiments the spraying solutions are prepared by using an additive blend comprising one or more additives selected from at least two groups of a) to d)
a) EO-PO block polymers having a HLB (hydrophilic-lipophilic-balance) value of 1, 2, 3, 4, 6, 8, 12, 13, 14, 15, 27 or 29 and an average molecular weight (AMW) between 1500 and 15000,
b) fatty acid methyl esters preferably C6-C10 methyl caproate-caprylate-caprate, C8-C10 methyl caprylate-caprate, C8 methyl caprylate, C10 methyl caprate, C12 methyl laurate, methyl coconate, palm kernel methyl ester, C14 methyl myristate, C16 methyl palmitate, C18 methyl stearate, methyl sunflowerate, palm oil methyl ester, methyl rapeate, methyl soyate and C18:1 methyl oleate more preferred C6-C10 fatty acid methyl ester, C12-C18 fatty acid methyl ester, soya fatty acid methyl ester, oleic acid methyl ester. More preferred are C6-C10 fatty acid methyl ester, soya fatty acid methyl ester, preferably a methyl soyate, and oleic acid methyl ester, preferably a C18:1 methyl oleate;
c) ethylenediamine tetra-functional PO/EO block polymers, preferably ethylenediamine tetra-functional PO/EO block polymers with a molecular weight (AMW) between 2000 and 20000, more preferred between 3000 and 18000, even more preferred they have a molecular weight of 3600, 4700 or 15000 and preferably have an HLB of 3 or 24,
d) fatty acid polyethylene esters preferably PEG 400 monolaurate, PEG 400 monooleate, PEG 400 dioleate more preferred is PEG 400 dioleate.

Additive blends comprising additives selected from two groups of a), b), c), or d) have an % v/v ratio of the two additives of the two groups of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1, wherein, if one or more additives of one group a), b), c) or d) is present, the total amount of all additives per group is calculated to determine the ratio.

Additive blends comprising additives selected from three groups of a), b), c), or d) have an % v/v ratio of the additives of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1 of the first group to the second group and a ratio of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1 of the first group to the third group, wherein, if one or more additives of one group a), b), c) or d) is present, the total amount of all additives per group is calculated to determine the ratio.

In some embodiments the spraying solutions are prepared by using an additive blend comprising one or more additives selected from the group of EO-PO block polymers having a HLB (hydrophilic-lipophilic-balance) value of 1, 2, 3, 4, 6, 8, 12, 13, 14, 15, 27 or 29 and an average molecular weight (AMW) between 1500 and 15000 and one or more additives selected from the groups of a) to c):
a) fatty acid methyl esters preferably C6-C10 methyl caproate-caprylate-caprate, C8-C10 methyl caprylate-caprate, C8 methyl caprylate, C10 methyl caprate, C12 methyl laurate, methyl coconate, palm kernel methyl ester, C14 methyl myristate, C16 methyl palmitate, C18 methyl stearate, methyl sunflowerate, palm oil methyl ester, methyl rapeate, methyl soyate and C18:1 methyl oleate more preferred C6-C10 fatty acid methyl ester, C12-C18 fatty acid methyl ester, soya fatty acid methyl ester, oleic acid methyl ester. More preferred are C6-C10 fatty acid methyl ester, soya fatty acid methyl ester, preferably a methyl soyate, and oleic acid methyl ester, preferably a C18:1 methyl oleate;
b) ethylenediamine tetra-functional PO/EO block polymers, preferably ethylenediamine tetra-functional PO/EO block polymers with a molecular weight (AMW) between 2000 and 20000, more preferred between 3000 and 18000, even more preferred they have a molecular weight of 3600, 4700 or 15000 and preferably have an HLB of 3 or 24,
c) fatty acid polyethylene esters preferably PEG 400 monolaurate, PEG 400 monooleate, PEG 400 dioleate more preferred is PEG 400 dioleate.

Additive blends comprising additives selected from the group of EO-PO block polymers of one group of a), b), or c) have an % v/v ratio of the additives of the two groups of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1, wherein, if one or more additives of one group is present, the total amount of all additives per group is calculated to determine the ratio.

Additive blends comprising additives selected from the group of EO-PO block polymers and two groups of a), b), or c) have an % v/v ratio of the additives of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1 of the EO-PO block polymers group to the second group and a ratio of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1 of the EO-PO block polymers group to the third group, wherein, if one or more additives of one group is present, the total amount of all additives per group is calculated to determine the ratio.

In some embodiments the spraying solutions are prepared by using an additive blend comprising one or more additives selected from the group of fatty acid methyl esters preferably C6-C10 methyl caproate-caprylate-caprate, C8-C10 methyl caprylate-caprate, C8 methyl caprylate, C10 methyl caprate, C12 methyl laurate, methyl coconate, palm kernel methyl ester, C14 methyl myristate, C16 methyl palmitate, C18 methyl stearate, methyl sunflowerate, palm oil methyl ester, methyl rapeate, methyl soyate and C18:1 methyl oleate more preferred C6-C10 fatty acid methyl ester, C12-C18 fatty acid methyl ester, soya fatty acid methyl ester, oleic acid methyl ester. More preferred are C6-C10 fatty acid methyl ester, soya fatty acid methyl ester, preferably a methyl soyate, and oleic acid methyl ester, preferably a C18:1 methyl oleate and one or more additives selected from the group of a) to c):
a) EO-PO block polymers having a HLB (hydrophilic-lipophilic-balance) value of 1, 2, 3, 4, 6, 8, 12, 13, 14, 15, 27 or 29 and an average molecular weight (AMW) between 1500 and 15000,
b) ethylenediamine tetra-functional PO/EO block polymers, preferably ethylenediamine tetra-functional PO/EO block polymers with a molecular weight (AMW) between 2000 and 20000, more preferred between 3000 and 18000 even more preferred they have a molecular weight of 3600, 4700 or 15000 and preferably they have an HLB of 3 or 24.
c) fatty acid polyethylene esters preferably PEG 400 monolaurate, PEG 400 monooleate, PEG 400 dioleate more preferred is PEG 400 dioleate.

Additive blends comprising additives selected from the group of fatty acid methyl esters and one group of a), b), or c) have an % v/v ratio of the additives of the two groups of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1, wherein, if one or more additives of one group is present, the total amount of all additives per group is calculated to determine the ratio.

Additive blends comprising additives selected from the group of fatty acid methyl esters and two groups of a), b), or c) have an % v/v ratio of the additives of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1 of the EO-PO block polymers group to the second group and a ratio of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1 of the EO-PO block polymers group to the third group, wherein, if one or more additives of one group is present, the total amount of all additives per group is calculated to determine the ratio.

In some embodiments the spraying solutions are prepared by using an additive blend comprising one or more additives selected from the group of ethylenediamine tetra-functional PO/EO block polymers, preferably ethylenediamine tetra-functional PO/EO block polymers with a molecular weight (AMW) between 2000 and 20000, more preferred between 3000 and 18000, even more preferred they have a molecular weight of 3600, 4700 or 15000 and have preferably an HLB of 3 or 24; and one or more additives selected from the group of a) to c):
a) EO-PO block polymers having a HLB (hydrophilic-lipophilic-balance) value of 1, 2, 3, 4, 6, 8, 12, 13, 14, 15, 27 or 29 and an average molecular weight (AMW) between 1500 and 15000
b) fatty acid methyl esters preferably C6-C10 methyl caproate-caprylate-caprate, C8-C10 methyl caprylate-caprate, C8 methyl caprylate, C10 methyl caprate, C12 methyl laurate, methyl coconate, palm kernel methyl ester, C14 methyl myristate, C16 methyl palmitate, C18 methyl stearate, methyl sunflowerate, palm oil methyl ester, methyl rapeate, methyl soyate and C18:1 methyl oleate more preferred C6-C10 fatty acid methyl ester, C12-C18 fatty acid methyl ester, soya fatty acid methyl ester, oleic acid methyl ester. More preferred are C6-C10 fatty acid methyl ester, soya fatty acid methyl ester, preferably a methyl soyate, and oleic acid methyl ester, preferably a C18:1 methyl oleate,
c) fatty acid polyethylene esters, preferably PEG 400 monolaurate, PEG 400 monooleate, PEG 400 dioleate more preferred is PEG 400 dioleate.

Additive blends comprising additives selected from the group of ethylenediamine tetra-functional PO/EO block polymers and one group of a), b), or c) have an % v/v ratio of the additives of the two groups of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1, wherein, if one or more additives of one group is present, the total amount of all additives per group is calculated to determine the ratio.

Additive blends comprising additives selected from the group of ethylenediamine tetra-functional PO/EO block polymers and two groups of a), b), or c) have an % v/v ratio of the additives of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1 of the EO-PO block polymers group to the second group and a ratio of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1 of the EO-PO block polymers group to the third group, wherein, if one or more additives of one group is present, the total amount of all additives per group is calculated to determine the ratio.

In some embodiments the spraying solutions are prepared by using an additive blend comprising one or more additives selected from the group of fatty acid polyethylene esters, preferably PEG 400 monolaurate, PEG 400 monooleate, PEG 400 dioleate even more preferred is PEG 400 dioleate and one or more additives selected from the group of a) to c):
a) EO-PO block polymers having a HLB (hydrophilic-lipophilic-balance) value of 1, 2, 3, 4, 6, 8, 12, 13, 14, 15, 27 or 29 and an average molecular weight (AMW) between 1500 and 15000
b) fatty acid methyl esters preferably C6-C10 methyl caproate-caprylate-caprate, C8-C10 methyl caprylate-caprate, C8 methyl caprylate, C10 methyl caprate, C12 methyl laurate, methyl coconate, palm kernel methyl ester, C14 methyl myristate, C16 methyl palmitate, C18 methyl stearate, methyl sunflowerate, palm oil methyl ester, methyl rapeate, methyl soyate and C18:1 methyl oleate more preferred C6-C10 fatty acid methyl ester, C12-C18 fatty acid methyl ester, soya fatty acid methyl ester, oleic acid methyl ester. More preferred are C6-C10 fatty acid methyl ester, soya fatty acid methyl ester, preferably a methyl soyate, and oleic acid methyl ester, preferably a C18:1 methyl oleate,
c) ethylenediamine tetra-functional PO/EO block polymers, preferably ethylenediamine tetra-functional PO/EO block polymers with a molecular weight (AMW) between 2000 and 20000, more preferred between 3000 and 18000, even more preferred they have a molecular weight of 3600, 4700 or 15000 and preferably they have an HLB of 3 or 24.

Additive blends comprising additives selected from the group of fatty acid polyethylene esters and one group of a), b), or c) have an % v/v ratio of the additives of the two groups of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1, wherein, if one or more additives of one group is present, the total amount of all additives per group is calculated to determine the ratio.

Additive blends comprising additives selected from the group of fatty acid polyethylene esters and two groups of a), b), or c) have an % v/v ratio of the additives of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1 of the EO-PO block polymers group to the second group and a ratio of between 10:1 and 1:1, 9:1 and 1:1, 8:1 and 1:1, 7:1 and 1:1, 6:1 and 1:1, 5:1 and 1:1, 4:1 and 1:1, 3:1 and 1:1, 2:1 and 1:1 or 1:1 of the EO-PO block polymers group to the third group, wherein, if one or more additives of one group is present, the total amount of all additives per group is calculated to determine the ratio.

Preferred examples are additive blends comprising additives selected from fatty acid polyethylene esters and one or both groups of a) or b):
a) EO-PO block polymers having a HLB (hydrophilic-lipophilic-balance) value of 1, 2, 3, 4, 6, 8, 12, 13, 14, 15, 27 or 29 and an average molecular weight (AMW) between 1500 and 15000,
b) fatty acid methyl esters preferably C6-C10 methyl caproate-caprylate-caprate, C8-C10 methyl caprylate-caprate, C8 methyl caprylate, C10 methyl caprate, C12 methyl laurate, methyl coconate, palm kernel methyl ester, C14 methyl myristate, C16 methyl palmitate, C18 methyl stearate, methyl sunflowerate, palm oil methyl ester, methyl rapeate, methyl soyate and C18:1 methyl oleate more preferred C6-C10 fatty acid methyl ester, C12-C18 fatty acid methyl ester, soya fatty acid methyl ester, oleic acid methyl ester. More preferred are C6-C10 fatty acid methyl ester, soya fatty acid methyl ester, preferably a methyl soyate, and oleic acid methyl ester, preferably a C18:1 methyl oleate.

Such additive blends may comprise additives selected from the group of fatty acid polyethylene esters and one group of a) or b) and have an % v/v ratio of the additives of the two groups of between 10:1 and 1:1, 9:1 and 1:1, 8:1 and 1:1, 7:1 and 1:1, 6:1 and 1:1, 5:1 and 1:1, preferably between 4:1 and 1:1, more preferred between 3:1 and 1:1, and even more preferred between 2:1 and 1:1 or 1:1, wherein, if one or more additives of one group is present, the total amount of all additives per group is calculated to determine the ratio.

Such additive blends may also comprise additives selected from the group of fatty acid polyethylene esters and both groups of a) and b) and have an % v/v ratio of the additives of the fatty acid polyethylene esters to EO-PO block polymers of between 10:1 and 1:1, 9:1 and 1:1, 8:1 and 1:1, 7:1 and 1:1, 6:1 and 1:1, 5:1 and 1:1, preferably between 4:1 and 1:1, more preferred between 3:1 and 1:1, and even more preferred between 2:1 and 1:1 or 1:1 and have an % v/v ratio of the additives of the fatty acid polyethylene esters to fatty acid methyl esters of between 10:1 and 1:1, 9:1 and 1:1, 8:1 and 1:1, 7:1 and 1:1, 6:1 and 1:1, 5:1 and 1:1, preferably between 4:1 and 1:1, more preferred between 3:1 and 1:1, and even more preferred between 2:1 and 1:1 or 1:1, wherein, if one or more additives of one group is present, the total amount of all additives per group is calculated to determine the ratio.

In some embodiments the spraying solutions are prepared by using an additive blend comprising one or more additives selected from the group of ethoxylated triglycerides, preferably selected from ethoxylated soybean oil with POE 10, 30, 42 or 60, ethoxylated castor oil with POE 2.5, 5, 7, 16, 18, 20, 25, 30, 33, 35, 36, 40, 44, 60 or 200, ethoxylated rapeseed oil with POE 30 and more preferred selected from soybean oil POE 10, soybean oil POE 30, castor oil POE 16, castor oil POE 40, most preferred is soybean oil POE 30, and one or more additives selected from the group of a) to d):
a) EO-PO block polymers having a HLB (hydrophilic-lipophilic-balance) value of 1, 2, 3, 4, 6, 8, 12, 13, 14, 15, 27 or 29 and an average molecular weight (AMW) between 1500 and 15000,
b) fatty acid polyethylene esters, preferably PEG 400 monolaurate, PEG 400 monooleate, PEG 400 dioleate even more preferred is PEG 400 dioleate,
c) alkoxylated alcohols, preferably having an HLB value of 9 or 12 or POP 31 POE 31 butyl alcohol,
d) alkyl naphthalene sulfonates, preferably dibutyl naphthalene sulfonate, more preferred the sodium salt of dibutyl naphthalene sulfonate.

Additive blends comprising additives selected from the group of ethoxylated triglycerides and one group of a), b), c) or b) have an % v/v ratio of the additives of the two groups of between 10:1 and 1:1, 9:1 and 1:1, 8:1 and 1:1, 7:1 and 1:1, 6:1 and 1:1, 5:1 and 1:1, preferably between 4:1 and 1:1, more preferred between 3:1 and 1:1, and even more preferred between 2:1 and 1:1 or 1:1, wherein, if one or more additives of the group of ethoxylated triglycerides and/or group a), b), c) or d) is present, the total amount of all additives per group id calculated to determine the ratio.

Additive blends comprising additives selected from the group of ethoxylated triglycerides and at least two groups of a), b), c) or b) have an % v/v ratio of the additives of between 10:1 and 1:1, 9:1 and 1:1, 8:1 and 1:1, 7:1 and 1:1, 6:1 and 1:1, 5:1 and 1:1, preferably between 4:1 and 1:1, more preferred between 3:1 and 1:1, and even more preferred between 2:1 and 1:1 or 1:1 of the ethoxylated triglycerides group to the second group and a ratio of between 10:1 and 1:1, 9:1 and 1:1, 8:1 and 1:1, 7:1 and 1:1, 6:1 and 1:1, 5:1 and 1:1, preferably between 4:1 and 1:1, more preferred between 3:1 and 1:1, and even more preferred between 2:1 and 1:1 or 1:1 of the ethoxylated triglycerides group to the third group, wherein, if one or more additives of one group is present, the total amount of all additives per group is calculated to determine the ratio.

Examples for such blends are additive blends comprising
a) additives selected from ethoxylated triglycerides and the group of EO-PO block polymers as second group, and
b) additive blends comprising additives selected from ethoxylated triglycerides and the group of fatty acid polyethylene esters as second group, and
c) additive blends comprising additives selected from ethoxylated triglycerides and the group of alkyl naphthalene sulfonates as second group, and
d) additive blends comprising additives selected from ethoxylated triglycerides and the group of fatty acid polyethylene esters as second group and EO-PO block polymers as third group, and
e) additive blends comprising additives selected from ethoxylated triglycerides and the group of fatty acid polyethylene esters as second group and alkoxylated alcohols as third group, and
f) additive blends comprising additives selected from ethoxylated triglycerides and the group of alkyl naphthalene sulfonates as second group and alkoxylated alcohols as third group.

In one embodiment the additive blend comprises one or more additives selected from the group of EO/PO/EO block polymers having a HLB (hydrophilic-lipophilic-balance) value of 1, 2, 3, 4, 6, 8, 12, 13, 14, 15, 27 or 29, preferably an EO/PO/EO block polymer with an HLB value of 2, 12, or 15, more preferred with an HLB value of 2 or 12 and even more preferred with an HLB value of 2, and an average molecular weight between 1500 and 15000
and
one or more additives selected from the group of PO/EO/PO block polymers with an HLB value of 4, 6, 8, or 12, preferably with an HLB value of 4,
and an average molecular weight between 1500 and 4000, preferably selected from 1950, 2150, 2650, 3100, or 3600, more preferred they have an average molecular weight of 3100, wherein the % v/v ratio of the EO/PO/EO block polymers and the PO/EO/PO block polymers is between 10:1 and 1:1,
and if one or more additives of the group of EO/PO/EO block polymers and/or the group of PO/EO/PO block polymers is present, the total amount of all additives per group is calculated to determine the ratio,
wherein the % v/v ratio of the EO/PO/EO block polymers and the PO/EO/PO block polymers is between 100:1 and 1:1, 90:1 and 1:1, 80:1 and 1:1, 70:1 and 1:1, 60:1 and 1:1, 50:1 and 1:1, 40:1 and 1:1, 30:1 and 1:1, 20:1 and 1:1, 10:1 and 1:1, and if one or more additives of the group of EO-PO block polymers and/or the group of PO/EO/PO block polymers is present, the total amount of all additives per group is calculated to determine the ratio.

In one embodiment the additive blend comprises 1 to 99%, 5 to 99%, 10 to 99%, 15 to 99%, 20 to 99%, 25 to 99%, 30 to 99%, 35 to 99%, 40 to 99%, 45 to 99%, 50 to 99%, 55 to 99%, 60 to 99%, 65 to 99%, 70 to 99%, 75 to 99%, 80 to 99%, 85 to 99%, 90 to 99%, 95 to 99% v/v of a first additive selected from: EO/PO/EO block polymers with an HLB value of 1, 2, 3, 12, 13, 14, 15, 27 or 29, and 1 to 5%, 1 to 10%, 1 to 15%, 1 to 20%, 1 to 25%, 1 to 30%, 1 to 35%, 1 to 40%, 1 to 45%, 1 to 50%, 1 to 55%, 1 to 60%, 1 to 65%, 1 to 70%, 1 to 75%, 1 to 80%, 1 to 85%, 1 to 90%, 1 to 95% v/v of one or more additive selected from
EO/PO/EO block polymers with an HLB value of 1, 2, 3, 12, 13, 14, 15, 27 or 29, PO/EO/PO block polymers with an HLB value of 4, 6, 8, 12 or 15, C6-C10 fatty acid methyl ester, C12-C18 fatty acid methyl ester, soya fatty acid methyl ester, oleic acid methyl ester, ethylenediamine tetra-functional PO/EO block polymer with an molecular weight of 3600, 4700 or 15000, C8-C10 alkylpolyglycosides, PEG 400 monolaurate, PEG 400 monooleate, PEG 400 dioleate, soybean oil POE 10, castor oil POE 16, castor oil POE 40, alkoxylated alcohol HLB 9 or 12, butyl alcohol POP 31 POE 31, dibutyl naphthalene sulfonate, or a lignosulfonatesC8-C10 alkylpolyglycoside blend, wherein the selected additives are not identical and the % v/v of all components add up to 100%.

The first additive is preferably an EO/PO/EO block polymer with an HLB value of 2, 12, or 15, more preferred with an HLB value of 2 or 12 and even more preferred with an HLB value of 2. In a preferred embodiment at least one of the further additives is a EO/PO/EO block polymer with an HLB value of 2, 12, or 15 a PO/EO/PO block polymer with an HLB value of 4, C6-C10 fatty acid methyl ester, a C12-C18 fatty acid methyl ester, a soya fatty acid methyl ester, an oleic acid methyl ester, a ethylenediamine tetra-functional PO/EO block polymer with a molecular weight of 15000, a C8-C10 alkylpolyglycosides, a PEG 400 dioleate, a soybean oil POE 10, an alkoxylated alcohol with an HLB value of 12, or a dibutyl naphthalene sulfonate.

In a more preferred embodiment, at least one of the further additives is a EO/PO/EO block polymer with an HLB value 2, or 12, a C6-C10 fatty acid methyl ester, a C12-C18 fatty acid methyl ester, a soya fatty acid methyl ester, an oleic acid methyl ester, a PEG 400 dioleate, a soybean oil POE 10, or a dibutyl naphthalene sulfonate.

In another embodiment at least one of the further additives is a EO/PO/EO block polymer with an HLB value 2 or 12, a C6-C10 fatty acid methyl ester, a C12-C18 fatty acid methyl ester, soya fatty acid methyl ester, a oleic acid methyl ester, a PEG 400 dioleate, a soybean oil POE 10, or a dibutyl naphthalene sulfonate.

At least one of the further additives is preferably
an EO/PO/EO block polymer with an HLB value of 2, 12, or 15, preferably with an HLB value of 2 or 12, or a PO/EO/PO block polymer with an HLB value of 4, 6, 8, or 12, preferably with an HLB value of 4, or a PEG 400 monolaurate, PEG 400 monooleate, PEG 400 dioleate, preferably a PEG 400 dioleate, or a soybean oil POE 10, castor oil POE 16, castor oil POE 40, preferably a soybean oil POE 10, or an alkoxylated alcohol with an HLB value of 9 or 12, preferably with an HLB value of 12, or a dibutyl naphthalene sulfonate.

Further preferred additive blends comprise 1 to 99%, 5 to 99%, 10 to 99%, 15 to 99%, 20 to 99%, 25 to 99%, 30 to 99%, 35 to 99%, 40 to 99%, 45 to 99%, 50 to 99%, 55 to 99%, 60 to 99%, 65 to 99%, 70 to 99%, 75 to 99%, 80 to 99%, 85 to 99%, 90 to 99%, 95 to 99% v/v of an EO/PO/EO block polymer with an HLB value of 2, 12, or 15, more preferred with an HLB value of 2 or 12 and even more preferred with an HLB value of 2 and an amount of 1 to 5%, 1 to 10%, 1 to 15%, 1 to 20%, 1 to 25%, 1 to 30%, 1 to 35%, 1 to 40%, 1 to 45%, 1 to 50%, 1 to 55%, 1 to 60%, 1 to 65%, 1 to 70%, 1 to 75%, 1 to 80%, 1 to 85%, 1 to 90%, 1 to 95% v/v of an PO/EO/PO block polymer with an HLB value of 4, 6, 8, 12 or 15, preferably with an HLB value of 4, show low phytotoxic effects.

Further exemplary additive blends suitable to produce spraying solutions of the invention are Additive Blends 1 to 7:

### Additive Blend 1:

Additive Blend 1 comprises 1 to 99% v/v of at least one additive selected from: soybean oil POE 10, castor oil POE 16 and castor oil POE 40, preferably at least one or all is soybean oil POE 10, and 1 to 99% v/v of at least one additive selected from: PEG 400 monolaurate, PEG 400 monooleate and PEG 400 dioleate, preferably at least one or all is PEG 400 dioleate, wherein the % v/v of both components add up to 100%.

A preferred variant of Additive Blend 1 comprises 20 to 80% v/v of at least one additive selected from: soybean oil POE 10, castor oil POE 16 and castor oil POE 40, preferably at least one or all is soybean oil POE 10, and 20 to 80% v/v of at least one additive selected from: PEG 400 monolaurate, PEG 400 monooleate and PEG 400 dioleate, preferably at least one or all is PEG 400 dioleate, wherein the % v/v of all components add up to 100%.

A more preferred variant of Additive Blend 1 comprises 40 to 60% v/v of at least one additive selected from: soybean oil POE 10, castor oil POE 16 and castor oil POE 40, at least one or all is soybean oil POE 10, and 40 to 60% v/v of at least one additive selected from: PEG 400 monolaurate, PEG 400 monooleate and PEG 400 dioleate, preferably at least one or all is PEG 400 dioleate, wherein the % v/v of both components add up to 100%.

In one variant, Additive Blend 1 comprises 45 to 55% v/v of at least one additive selected from: soybean oil POE 10, castor oil POE 16 and castor oil POE 40, preferably at least one or all is soybean oil POE 10, and 45 to 55% v/v of at least one additive selected from: PEG 400 monolaurate, PEG 400 monooleate and PEG 400 dioleate, preferably at least one or all is PEG 400 dioleate, wherein the % v/v of both components add up to 100%.

Additive Blend 1 comprises 5 to 60% v/v of at least one additive selected from: soybean oil POE 10, soybean oil POE 30, castor oil POE 16 and castor oil POE 40, preferably at least one or all is soybean oil POE 10, and 40 to 60% v/v of at least one additive selected from: PEG 400 monolaurate, PEG 400 monooleate and PEG 400 dioleate, preferably at least one or all is PEG 400 dioleate, wherein the % v/v of both components add up to 100%.

### Additive Blend 2:

Additive Blend 2 comprises 1 to 98% v/v of at least one additive selected from: soybean oil POE 10, castor oil POE 16 and castor oil POE 40, preferably at least one or all is soybean oil POE 10, and 1 to 98 v/v of at least one additive selected from: PEG 400 monolaurate, PEG 400 monooleate and PEG 400 dioleate, preferably at least one or all is PEG 400 dioleate and 1 to 98% v/v of at least one additive selected from: alkoxylated alcohol having an HLB value of 9 or 12, preferably at least one or all has an HLB value of 12, wherein the % v/v of all components add up to 100%.

A preferred variant of Additive Blend 2 comprises 5 to 94% v/v of at least one additive selected from: soybean oil POE 10, castor oil POE 16 and castor oil POE 40, preferably at least one or all is soybean oil POE 10, 5 to 89% v/v of at least one additive selected from: PEG 400 monolaurate, PEG 400 monooleate and PEG 400 dioleate, preferably at least one or all is PEG 400 dioleate, and 1 to 40% v/v of at least one additive selected from: alkoxylated alcohol having an HLB value of 9 or 12, preferably at least one or all has an HLB value of 12, wherein the % v/v of all components add up to 100%.

A more preferred variant of Additive Blend 2 comprises 30 to 89% v/v of at least one additive selected from: soybean oil POE 10, castor oil POE 16 and castor oil POE 40, preferably at least one or all is soybean oil POE 10, 10 to 40% v/v of at least one additive selected from: PEG 400 monolaurate, PEG 400 monooleate and PEG 400 dioleate, preferably at least one or all is PEG 400 dioleate, and 1 to 30% v/v of at least one additive selected from: alkoxylated alcohol having an HLB value of 9 or 12, preferably at least one or all has an HLB value of 12, alkoxylated alcohol having an HLB value of 9 or 12, wherein the % v/v of all components add up to 100%.

An even more preferred variant of Additive Blend 2 comprises 50 to 80% v/v of at least one additive selected from: soybean oil POE 10, castor oil POE 16 and castor oil POE 40, preferably at least one or all is soybean oil POE 10, 15 to 35% v/v of at least one additive selected from: PEG 400 monolaurate, PEG 400 monooleate and PEG 400 dioleate, preferably at least one or all is PEG 400 dioleate, and 5 to 25% v/v of at least one additive selected from: alkoxylated alcohol having an HLB value of 9 or 12, preferably at least one or all has an HLB value of 12, wherein the % v/v of all components add up to 100%.

In one variant Additive Blend 2 comprises 40 to 75% v/v of at least one additive selected from: soybean oil POE 10, castor oil POE 16 and castor oil POE 40, preferably at least one or all is soybean oil POE 10, 15 to 40% v/v of at least one additive selected from: PEG 400 monolaurate, PEG 400 monooleate and PEG 400 dioleate, preferably at least one or all is PEG 400 dioleate, and 10 to 20% v/v of at least one additive selected from: alkoxylated alcohol having an HLB value of 9 or 12, preferably at least one or all has an HLB value of 12 wherein the % v/v of both components add up to 100%.

### Additive Blend 3:

Additive Blend 3 comprises 1 to 99% v/v of at least one additive selected from: soybean oil POE 10, castor oil POE 16 and castor oil POE 40, preferably at least one or all is soybean oil POE 10, and 1 to 99% v/v of dibutyl naphthalene sulfonate, preferably sodium dibutyl naphthalene sulfonate, wherein the % v/v of all components add up to 100%.

A preferred variant of Additive Blend 3 comprises 40 to 90% v/v of at least one additive selected from: soybean oil POE 10, castor oil POE 16 and castor oil POE 40, preferably at least one or all is soybean oil POE 10, and 10 to 60% v/v of dibutyl naphthalene sulfonate, preferably sodium dibutyl naphthalene sulfonate, wherein the % v/v of all components add up to 100%.

An even more preferred variant of Additive Blend 3 comprises 55 to 80% v/v of at least one additive selected from: soybean oil POE 10, castor oil POE 16 and castor oil POE 40, preferably at least one or all is soybean oil POE 10, and 20 to 45% v/v dibutyl naphthalene sulfonate, preferably sodium dibutyl naphthalene sulfonate, wherein the % v/v of all components add up to 100%.

In one variant Additive Blend 3 comprises 60 to 75% v/v of at least one additive selected from: soybean oil POE 10, castor oil POE 16 or castor oil POE 40, preferably a soybean oil POE 10, and 25 to 40% v/v dibutyl naphthalene sulfonate, preferably sodium dibutyl naphthalene sulfonate, wherein the % v/v of all components add up to 100%.

### Additive Blend 4:

Additive Blend 4 comprises 1 to 98% v/v of at least one additive selected from: soybean oil POE 10, castor oil POE 16 and castor oil POE 40, preferably at least one or all is soybean oil POE 10, and 1 to 98% v/v of at least one additive selected from: PEG 400 monolaurate, PEG 400 monooleate and PEG 400 dioleate, preferably at least one or all is PEG 400 dioleate, and 1 to 98% v/v of at least one additive selected from: EO/PO/EO block polymers with an HLB value of 1, 2, 3, 12, 13, 14, 15, 27 and 29, preferably at least one or all have an HLB value of 2 or 12, more preferred at least one or all have a HLB value of 2, wherein the % v/v of all components add up to 100%.

In a preferred variant of Additive Blend 4 comprises 1 to 90% v/v of at least one additive selected from: soybean oil POE 10, castor oil POE 16 and castor oil POE 40, preferably at least one or all is soybean oil POE 10, and 10 to 88% v/v of at least one additive selected from: PEG 400 monolaurate, PEG 400 monooleate and PEG 400 dioleate, preferably at least one or all is PEG 400 dioleate, and 10 to 88% v/v of at least one additive selected from: EO/PO/EO block polymers with an HLB value of 1, 2, 3, 12, 13, 14, 15, 27 and 29, preferably at least one or all have an HLB value of 2 or 12, more preferred at least one or all have a HLB value of 2, wherein the % v/v of all components add up to 100%.

An equally preferred variant of Additive Blend 4 comprises 1 to 70% v/v of at least one additive selected from: soybean oil POE 10, castor oil POE 16 and castor oil POE 40, preferably at least one or all is soybean oil POE 10, and 20 to 80% v/v of at least one additive selected from: PEG 400 monolaurate, PEG 400 monooleate and PEG 400 dioleate, preferably at least one or all is PEG 400 dioleate, and 10 to 79% v/v of at least two additives selected from: EO/PO/EO block polymers with an HLB value of 1, 2, 3, 12, 13, 14, 15, 27 and 29, preferably at least one has an HLB value of 12 and at least one has an HLB value of 2, wherein the % v/v of all components add up to 100%.

A preferred variant of Additive Blend 4 comprises 40 to 60% v/v of at least one additive selected from: soybean oil POE 10, castor oil POE 16 and castor oil POE 40, preferably at least one or all is soybean oil POE 10, and 15 to 40% v/v of at least one additive selected from: PEG 400 monolaurate, PEG 400 monooleate and PEG 400 dioleate, preferably at least one or all is PEG 400 dioleate, and 15 to 30% v/v of an EO/PO/EO block polymers with an HLB value of 12 and 15 to 30% v/v of an EO/PO/EO block polymers with an HLB value of 2, wherein the % v/v of all components add up to 100%.

### Additive Blend 5:

Additive Blend 5 comprises 1 to 95% v/v of at least one additive selected from: soybean oil POE 10, castor oil POE 16 and castor oil POE 40, preferably at least one or all is soybean oil POE 10, and 1 to 95% v/v of at least one additive selected from: PEG 400 monolaurate, PEG 400 monooleate and PEG 400 dioleate, preferably at least one or all is PEG 400 dioleate, and 1 to 95% v/v dibutyl naphthalene sulfonate, preferably sodium dibutyl naphthalene sulfonate, and 1 to 95% v/v of at least one additive selected from: alkoxylated alcohol having an HLB value of 9 or 12, preferably at least one or all has an HLB value of 12, and 1 to % v/v at least one selected from: EO/PO/EO block polymers with an HLB value of 1, 2, 3, 12, 13, 14, 15, 27 and 29, preferably at least one has an HLB value of 2, 12, or 15, more preferred at least one has an HLB value of 2, wherein the % v/v of all components add up to 100%.

A preferred variant of Additive Blend 5 comprises 10 to 78% v/v of at least one additive selected from: soybean oil POE 10, castor oil POE 16 and castor oil POE 40, preferably at least one or all is soybean oil POE 10, and 1 to 30% v/v of at least one additive selected from: PEG 400 monolaurate, PEG 400 monooleate and PEG 400 dioleate, preferably at least one or all is PEG 400 dioleate, and 10 to 78% v/v dibutyl naphthalene sulfonate, preferably sodium dibutyl naphthalene sulfonate, and 10 to 78% v/v of at least one additive selected from: alkoxylated alcohol having an HLB value of 9 or 12, preferably at least one or all has an HLB value of 12, and 1 to 30% v/v at least one selected from: EO/PO/EO block polymers with an HLB value of 1, 2, 3, 12, 13, 14, 15, 27 and 29, preferably at least one has an HLB value of 2, 12, or 15, more preferred at least one has an HLB value of 2, wherein the % v/v of all components add up to 100%.

A more preferred variant of Additive Blend 5 comprises 20 to 78% v/v of at least one additive selected from: soybean oil POE 10, castor oil POE 16 and castor oil POE 40, preferably at least one or all is soybean oil POE 10, and 1 to 20% v/v at least one additive selected from: PEG 400 monolaurate, PEG 400 monooleate and PEG 400 dioleate, preferably at least one or all is PEG 400 dioleate, and 10 to 40% v/v dibutyl naphthalene sulfonate, preferably sodium dibutyl naphthalene sulfonate, and 10 to 40% of at least one additive selected from: alkoxylated alcohol having an HLB value of 9 or 12, preferably at least one or all has an HLB value of 12, and 1 to 20% v/v at least one selected from: EO/PO/EO block polymers with an HLB value of 1, 2, 3, 12, 13, 14, 15, 27 and 29, preferably at least one has an HLB value of 2, 12, or 15, more preferred at least one has an HLB value of 2.

### Additive Blend 6:

Additive Blend 6 comprises 1 to 97% v/v of at least one additive selected from: soybean oil POE 10, castor oil POE 16 and castor oil POE 40, preferably at least one or all is soybean oil POE 10, and 1 to 97% v/v dibutyl naphthalene sulfonate, preferably sodium dibutyl naphthalene sulfonate, and 1 to 97% v/v an alkoxylated alcohol having an HLB value of 9 or 12, preferably at least one or all has an HLB value of 12, wherein the % v/v of all components add up to 100%.

A preferred variant of Additive Blend 6 comprises 20 to 90% v/v of at least one additive selected from: soybean oil POE 10, castor oil POE 16 and castor oil POE 40, preferably at least one or all is soybean oil POE 10, and 5 to 50% v/v dibutyl naphthalene sulfonate, preferably sodium dibutyl naphthalene sulfonate, and 5 to 50% v/v an alkoxylated alcohol having an HLB value of 9 or 12, preferably at least one or all has an HLB value of 12, wherein the % v/v of all components add up to 100%.

A more preferred variant of Additive Blend 6 comprises 30 to 70% v/v of at least one additive selected from: soybean oil POE 10, castor oil POE 16 and castor oil POE 40, preferably at least one or all is soybean oil POE 10, and 15 to 40% v/v dibutyl naphthalene sulfonate, preferably sodium dibutyl naphthalene sulfonate, and 15 to 40% v/v alkoxylated alcohol having an HLB value of 9 or 12, preferably at least one or all has an HLB value of 12, wherein the % v/v of all components add up to 100%.

### Additive Blend 7:

Additive Blend 7 comprises 1 to 97% v/v of at least one additive selected from: EO/PO/EO block polymers with an HLB value of 1, 2, 3, 12, 13, 14, 15, 27 and 29, preferably at least one or all have an HLB value of 2, or 12, more preferred at least one or all have a HLB value of 2, and 1 to 97% v/v of at least one additive selected from: PEG 400 monolaurate, PEG 400 monooleate and PEG 400 dioleate, preferably at least one or all is PEG 400 dioleate, and 1 to 97% v/v of at least one additive selected from:
soybean oil POE 10, castor oil POE 16 and castor oil POE 40, preferably at least one or all is soybean oil POE 10, wherein the % v/v of all components add up to 100%.

A preferred variant of Additive Blend 7 comprises 5 to 50% v/v of at least one additive selected from: EO/PO/EO block polymers with an HLB value of 1, 2, 3, 12, 13, 14, 15, 27 and 29, preferably at least one or all have an HLB value of 2 or 12, more preferred at least one or all have a HLB value of 2, and 5 to 50% v/v of at least one additive selected from: PEG 400 monolaurate, PEG 400 monooleate and PEG 400 dioleate, preferably at least one or all is PEG 400 dioleate, and 20 to 90% v/v of at least one additive selected from: soybean oil POE 10, castor oil POE 16 and castor oil POE 40, preferably at least one or all is soybean oil POE 10, wherein the % v/v of all components add up to 100%.

A more preferred variant of Additive Blend 7 comprises 10 to 30% v/v of at least one additive selected from: EO/PO/EO block polymers with an HLB value of 1, 2, 3, 12, 13, 14, 15, 27 and 29, preferably at least one or all have an HLB value of 2 or 12, more preferred at least one or all have a HLB value of 2, and 10 to 30% v/v of at least one additive selected from: PEG 400 monolaurate, PEG 400 monooleate and PEG 400 dioleate, preferably at least one or all is PEG 400 dioleate, and 40 to 80% v/v of at least one additive selected from: soybean oil POE 10, castor oil POE 16 and castor oil POE 40, preferably at least one or all is soybean oil POE 10, wherein the % v/v of all components add up to 100%.

The additive blends for preparing the spraying liquids may comprise further components like water, without changing the relative amounts of additives in blends to each other as having been described above.

The amount of water is usually kept low in order to keep the total volume of the additive blend low. Usual amounts of water are between 1 to 70% v/v of the total volume. Preferably the amount of water is between 5 to 65% v/v, 10 to 60% v/v, 15 to 60% v/v, 20 to 60% v/v, 25 to 60% v/v, 30 to 60% v/v, or between 1 to 60% v/v, 1 to 55% v/v, 1 to 50% v/v, 1 to 45% v/v, 1 to 40% v/v, 1 to 35% v/v or 1 to 30% v/v.

For example, a variant of Additive Blend 7 comprises 40 to 70% v/v of at least one additive selected from: soybean oil POE 10, castor oil POE 16 or castor oil POE 40, preferably a soybean oil POE 10, and 10 to 50% v/v dibutyl naphthalene sulfonate, preferably sodium dibutyl naphthalene sulfonate, and 10 to 50% v/v of water, wherein the % v/v of all components add up to 100%

In one variant Additive Blend 3 comprises 40 to 70% v/v at least one additive selected from: soybean oil POE 10, castor oil POE 16 or castor oil POE 40, preferably a soybean oil POE 10, 10 to 50% v/v dibutyl naphthalene sulfonate, preferably sodium dibutyl naphthalene sulfonate, and 15 to 35% v/v of water, wherein the % v/v of all components add up to 100%.

In one variant Additive Blend 5 comprises 15 to 30% v/v of at least one additive selected from: soybean oil POE 10, castor oil POE 16 and castor oil POE 40, preferably at least one or all is soybean oil POE 10, and 1 to 10% v/v of at least one additive selected from: PEG 400 monolaurate, PEG 400 monooleate and PEG 400 dioleate, preferably at least one or all is PEG 400 dioleate, and 1 to 20% v/v dibutyl naphthalene sulfonate, preferably sodium dibutyl naphthalene sulfonate, and 15 to 25% v/v of at least one additive selected from: alkoxylated alcohol having an HLB value of 9 or 12, preferably at least one or all has an HLB value of 12, and 1 to 10% v/v at least one selected from: EO/PO/EO block polymers with an HLB value of 1, 2, 3, 12, 13, 14, 15, 27 and 29, preferably at least one has an HLB value of 2, 12, or 15, more preferred at least one has an HLB value of 2, and 15 to 35% v/v of water, wherein the % v/v of all components add up to 100%.

Additive Blend 6 comprises 30 to 60% v/v of at least one additive selected from: soybean oil POE 10, castor oil POE 16 and castor oil POE 40, preferably at least one or all is soybean oil POE 10, and 1 to 20% v/v dibutyl naphthalene sulfonate, preferably sodium dibutyl naphthalene sulfonate, and 20 to 30% v/v an alkoxylated alcohol having an HLB value of 9 or 12, preferably at least one or all has an HLB value of 12, and 15 to 35% v/v of water, wherein the % v/v of all components add up to 100%.

Further preferred Additive Blends comprising or not comprising water are disclosed in the Examples.

In some embodiments the spraying liquids comprise viable cells of the phylum Pseudomonadales, preferably of the genus Pseudomonas, even more preferred of the species Pseudomonas fluorescens or Pseudomonas chlororaphis, and most preferred of the strain Pseudomonas fluorescens A506.

Spraying liquids comprising viable cells of the phylum Pseudomonadales and additive blends for preparation of these spraying liquids may comprise all additives described above, but less preferred additives are: EO/PO/EO Block Polymers with an HLB value of 29, PO/EO/PO Block Polymers with an HLB value of 4, PEG 400 monolaurate and PEG 400 monooleate. Preferred additives are C6-C10 fatty acid methyl ester, C12-C18 fatty acid methyl ester, soya fatty acid methyl ester, oleic acid methyl ester, PEG 400 dioleate, soybean oil POE 10, and polyquaternium-7, more preferred C6-C10 fatty acid methyl ester, oleic acid methyl ester, PEG 400 dioleate and soybean oil POE 10.

Preferred additive blends for Pseudomonadales are Additive Blends 1, 2 and 7.

The spraying liquids for spores or viable cells may comprise further components. These further components may be components present in the agrochemical formulation of the pesticidal microorganism used to prepare the spraying liquid via dilution with water.

Such further components can also be water miscible mineral oil or synthetic agrochemicals, like fungicides, insecticides, herbicides or plant growth regulators, which may be comprised by the agrochemical formulation of the pesticidal microorganisms or are used in a tank mix application by adding one or more additional agrochemical formulations comprising the one or more synthetic agrochemicals.

In addition to synthetic agrochemicals, or alternatively to synthetic agrochemicals, the spraying liquids my comprise pesticidal natural compounds as further component. These pesticidal natural compound can be any naturally produced compound which has fungicidal, insecticidal, miticidal, nematocidal, herbicidal or plant growth regulating activity.

If agrochemical formulations of synthetic agrochemicals and/or pesticidal natural compounds are used, the spraying liquids will also comprise the formulation components of the respective synthetic agrochemical and/or pesticidal natural compound as further components.

The spraying liquids are preferably prepared by adding an additive blend to a water diluted agropesticidal formulation comprising the pesticidal microorganism. Usually the total amount of spraying liquids per hectare is between 1000 L/ha and 100L/ha, 600 L/ha and 100L/ha, 400 L/ha and 100L/ha, 200L/ha and 100L/ha or between 1000 L/ha and 600 L/ha, 1000 L/ha and 400 L/ha or 1000 L/ha and 200L/ha, or between 600 L/ha and 200L/ha, 600 L/ha and 400 L/ha. Wherein a higher amount of spraying liquid is usually selected to achieve an even and complete coverage of the plants surface when the spraying liquid is applied to the plant or plant parts of interest.

Accordingly, the invention comprises also kits of at least two parts to prepare a spraying liquid suitable for spraying plants or for preparing a seed treatment composition for treating seeds of plants, wherein the bacterial spores or vegetative cells are provided in a first concentrated form and at least one of the additive blends is provided in at least one further concentrated form and wherein the relative amounts of the bacterial spores or vegetative cells and the relative amounts of the additive blends in the kit are adapted to provide the amounts described for the spraying liquids described above, preferably the spraying liquids comprise one of additive blends 1 to 7. Preferably the spraying liquids produced by using the kit of at least two parts are prepared to be used in a method to control phytopathogenic fungi.

The spraying liquids are, dependent on the pesticidal microorganism present in the spraying liquid, useful in methods of suppressing or preventing or reducing infection of plants with phytopathogenic fungi, bacteria or insects.

The spraying liquids are employed by treating the fungi, the bacteria, insects the plants, young plants, like seedlings, rooted/unrooted cuttings, plants derived from cell-culture or plant propagation materials, such as seeds; seedlings, cuttings, soil, surfaces, materials, or rooms to be protected from fungal or bacterial attack with an effective amount of the pesticidal microorganism. The application can be carried out both before and after the infection of the plants, plant propagation materials, such as seeds; soil, surfaces, materials or rooms by the fungi and/or bacteria.

The spraying liquids can be employed in pre-harvest and in post-harvest applications.

The term “effective amount” denotes an amount of the spraying liquids, which is sufficient for preventing or reducing the infection of a plant by harmful fungi or bacteria, but which does not result in a substantial damage to the treated plant, young plants, like seedlings, rooted/unrooted cuttings, plants derived from cell-culture or plant propagation material, such as seeds. Such an amount can vary in a broad range and is dependent on various factors, such as the fungal or bacterial species to be controlled, the treated plant species, the climatic conditions and the specific mixture used. Plant propagation materials may be treated at or before planting or transplanting.

The user applies the spraying liquids according to the invention usually from a predosage device, a knapsack sprayer, a spray tank, a spray plane, or an irrigation system. Usually, 20 to 2000 liters, preferably between 50, 100, 200, 300, 400, 500, 600, 700, 800, 900 to 1000 liters, of the spraying liquid is applied per hectare of agricultural useful area.

One further embodiment of the invention are methods to control phytopathogenic fungi or phytopathogenic bacteria wherein an additive blend described above or a kit of at least two parts as described above is used to prepare a spraying liquid for spraying plants or for preparing a seed treatment composition for treating seeds and the spraying liquid for spraying plants is sprayed on plants or the plant seeds are treated with the seed treatment composition for treating seeds.

The spraying liquids are preferably useful in the control of phytopathogenic fungi and/or bacteria on various cultivated plants, such as cereals, e. g. wheat, rye, barley, triticale, oats, or rice; beet, e. g. sugar beet or fodder beet; fruits, e. g. pomes (apples, pears, etc.), stone fruits (e.g. plums, peaches, almonds, cherries), or soft fruits, also called berries (strawberries, raspberries, blackberries, gooseberries, etc.); leguminous plants, e. g. lentils, peas, alfalfa, or soybeans; oil plants, e. g. oilseed rape, mustard, olives, sunflowers, coconut, cocoa beans, castor oil plants, oil palms, ground nuts, or soybeans; cucurbits, e. g. squashes, cucumber, or melons; fiber plants, e. g. cotton, flax, hemp, or jute; citrus fruits, e. g. oranges, lemons, grapefruits, or mandarins; vegetables, e. g. spinach, lettuce, asparagus, cabbages, carrots, onions, tomatoes, potatoes, cucurbits, or paprika; lauraceous plants, e. g. avocados, cinnamon, or camphor; energy and raw material plants, e. g. corn, soybean, oilseed rape, sugar cane, or oil palm; corn; tobacco; nuts; coffee; tea; bananas; vines (table grapes and grape juice grape vines); hop; turf; sweet leaf (also called Stevia); natural rubber plants; or ornamental and forestry plants, e. g. flowers, shrubs, broad-leaved trees, or evergreens (conifers, eucalypts, etc.); on the plant propagation material, such as seeds; and on the crop material of these plants.

More preferably, the spraying liquids are used for controlling fungi and/or bacteria on crops, such as potatoes, sugar beets, tobacco, wheat, rye, barley, oats, rice, corn, cotton, soybeans, oilseed rape, legumes, sunflowers, coffee or sugar cane; fruits; vines; table grapes, grapes for wine making or table grapes, ornamentals; or vegetables, such as cucumbers, tomatoes, pepper, beans or squashes.

According to the invention all of the above cultivated plants are understood to comprise all species, subspecies, variants and/or hybrids which belong to the respective cultivated plants. Corn is also known as Indian corn or maize (Zea mays) which comprises all kinds of corn such as field corn and sweet corn. According to the invention all maize or corn subspecies and/or varieties are comprised, in particular flour corn (Zea mays var. amylacea), popcorn (Zea mays var. everta), dent corn (Zea mays var. indentata), flint corn (Zea mays var. indurata), sweet corn (Zea mays var. saccharata and var. rugosa), waxy corn (Zea mays var. ceratina), amylomaize (high amylose Zea mays varieties), pod corn or wild maize (Zea mays var. tunicata) and striped maize (Zea mays var. japonica).

The term “cultivated plants” is to be understood as including plants which have been modified by mutagenesis or genetic engineering to provide a new trait to a plant or to modify an already present trait. Mutagenesis includes random mutagenesis using X-rays or mutagenic chemicals, but also targeted mutagenesis to create mutations at a specific locus of a plant genome. Targeted mutagenesis frequently uses oligonucleotides or proteins like CRISPR/Cas, zinc-finger nucleases, TALENs or meganucleases. Genetic engineering usually uses recombinant DNA techniques to create modifications in a plant genome which under natural circumstances cannot readily be obtained by cross breeding, mutagenesis or natural recombination. Typically, one or more genes are integrated into the genome of a plant to add a trait or improve or modify a trait. These integrated genes are also referred to as transgenes, while plant comprising such transgenes are referred to as transgenic plants. The process of plant transformation usually produces several transformation events, which differ in the genomic locus in which a transgene has been integrated. Plants comprising a specific transgene on a specific genomic locus are usually described as comprising a specific “event”, which is referred to by a specific event name. Traits which have been introduced in plants or have been modified include herbicide tolerance, insect resistance, increased yield and tolerance to abiotic conditions, like drought.

Herbicide tolerance has been created by using mutagenesis and genetic engineering. Plants which have been rendered tolerant to acetolactate synthase (ALS) inhibitor herbicides by mutagenesis and breeding are e.g. available under the name Clearfield®. Herbicide tolerance to glyphosate, glufosinate, 2,4-D, dicamba, oxynil herbicides, like bromoxynil and ioxynil, sulfonylurea herbicides, ALS inhibitors and 4-hydroxyphenylpyruvate dioxygenase (HPPD) inhibitors, like isoxaflutole and mesotrione, has been created via the use of transgenes.

Transgenes to provide herbicide tolerance traits comprise: for tolerance to glyphosate: cp4 epsps, epsps grg23ace5, mepsps, 2mepsps, gat4601, gat4621, goxv247; for tolerance to glufosinate: pat and bar, for tolerance to 2,4-D: aad-1, aad-12; for tolerance to dicamba: dmo; for tolerance to oxynil herbicies: bxn; for tolerance to sulfonylurea herbicides: zm-hra, csr1-2, gm-hra, S4-HrA; for tolerance to ALS inhibitors: csr1-2; and for tolerance to HPPD inhibitors: hppdPF, W336, avhppd-03.

Transgenic corn events comprising herbicide tolerance genes include, but are not limited to, DAS40278, MON801, MON802, MON809, MON810, MON832, MON87411, MON87419, MON87427, MON88017, MON89034, NK603, GA21, MZHGOJG, HCEM485, VCO-Ø1981-5, 676, 678, 680, 33121, 4114, 59122, 98140, Bt10, Bt176, CBH-351, DBT418, DLL25, MS3, MS6, MZIR098, T25, TC 1507 and TC6275. Transgenic soybean events comprising herbicide tolerance genes include, but are not limited to, GTS 40-3-2, MON87705, MON87708, MON87712, MON87769, MON89788, A2704-12, A2704-21, A5547-127, A5547-35, DP356043, DAS44406-6, DAS68416-4, DAS-81419-2, GU262, SYHTØH2, W62, W98, FG72 and CV127. Transgenic cotton events comprising herbicide tolerance genes include, but are not limited to, 19-51a, 31707, 42317, 81910, 281-24-236, 3006-210-23, BXN10211, BXN10215, BXN10222, BXN10224, MON1445, MON1698, MON88701, MON88913, GHB119, GHB614, LLCotton25, T303-3 and T304-40. Transgenic canola events comprising herbicide tolerance genes are for example, but not excluding others, MON88302, HCR-1, HCN10, HCN28, HCN92, MS1, MS8, PHY14, PHY23, PHY35, PHY36, RF1, RF2 and RF3.

Transgenes to provide insect resistance preferably are toxin genes of Bacillus spp. and synthetic variants thereof, like cry1A, cry1Ab, cry1Ab-Ac, cry1Ac, cry1A. 105, cry1F, cry1Fa2, cry2Ab2, cry2Ae, mcry3A, ecry3.1Ab, cry3Bb1, cry34Ab1, cry35Ab1, cry9C, vip3A(a), vip3Aa20. In addition, transgenes of plant origin, such as genes coding for protease inhibitors, like CpTI and pinII, can be used. A further approach uses transgenes such as dvsnf7 to produce double-stranded RNA in plants.

Transgenic corn events comprising genes for insecticidal proteins or double stranded RNA include, but are not limited to, Bt10, Bt11, Bt176, MON801, MON802, MON809, MON810, MON863, MON87411, MON88017, MON89034, 33121, 4114, 5307, 59122, TC1507, TC6275, CBH-351, MIR162, DBT418 and MZIR098. Transgenic soybean events comprising genes for insecticidal proteins include, but are not limited to, MON87701, MON87751 and DAS-81419. Transgenic cotton events comprising genes for insecticidal proteins include, but are not limited to, SGK321, MON531, MON757, MON1076, MON15985, 31707, 31803, 31807, 31808, 42317, BNLA-601, Event1, COT67B, COT102, T303-3, T304-40, GFM CrylA, GK12, MLS 9124, 281-24-236, 3006-210-23, GHB119 and SGK321.

Cultivated plants with increased yield have been created by using the transgene athb17 (e.g. corn event MON87403), or bbx32 (e.g. soybean event MON87712).

Cultivated plants comprising a modified oil content have been created by using the transgenes: gm-fad2-1, Pj.D6D, Nc.Fad3, fad2-1A and fatb1-A (e.g. soybean events 260-05, MON87705 and MON87769).

Tolerance to abiotic conditions, such as drought, has been created by using the transgene cspB (corn event MON87460) and Hahb-4 (soybean event IND-ØØ41Ø-5).

Traits are frequently combined by combining genes in a transformation event or by combining different events during the breeding process resulting in a cultivated plant with stacked traits. Preferred combinations of traits are combinations of herbicide tolerance traits to different groups of herbicides, combinations of insect tolerance to different kind of insects, in particular tolerance to lepidopteran and coleopteran insects, combinations of herbicide tolerance with one or several types of insect resistance, combinations of herbicide tolerance with increased yield as well as combinations of herbicide tolerance and tolerance to abiotic conditions.

Plants comprising singular or stacked traits as well as the genes and events providing these traits are well known in the art. For example, detailed information as to the mutagenized or integrated genes and the respective events are available from websites of the organizations “International Service for the Acquisition of Agri-biotech Applications (ISAAA)” (http://www.isaaa.org/gmapprovaldatabase) and the “Center for Environmental Risk Assessment (CERA)” (http://cera-gmc.org/GMCropDatabase). Further information on specific events and methods to detect them can be found for canola events MS1, MS8, RF3, GT73, MON88302, KK179 in WO01/031042, WO01/041558, WO01/041558, WO02/036831, WO11/153186, WO13/003558; for cotton events MON1445, MON 15985, MON531 (MON15985), LLCotton25, MON88913, COT102, 281-24-236, 3006-210-23, COT67B,

GHB614, T304-40, GHB119, MON88701, 81910 in WO02/034946, WO02/100163, WO02/100163, WO03/013224, WO04/072235, WO04/039986, WO05/103266, WO05/103266, WO06/128573, WO07/017186, WO08/122406, WO08/151780, WO12/134808, WO13/112527; for corn events GA21, MON810, DLL25, TC1507, MON863, MIR604, LY038, MON88017, 3272, 59122, NK603, MIR 162, MON89034, 98140, 32138, MON87460, 5307, 4114, MON87427, DAS40278, MON87411, 33121, MON87403, MON87419 in WO98/044140, US02/102582, US03/126634, WO04/099447, WO04/011601, WO05/103301, WO05/061720, WO05/059103, WO06/098952, WO06/039376, US2007/292854, WO07/142840, WO07/140256, WO08/112019, WO09/103049, WO09/111263, WO10/077816, WO11/084621, WO11/062904, WO11/022469, WO13/169923, WO14/116854, WO15/053998, WO15/142571; for potato events

E12, F10, J3, J55, V11, X17, Y9 in WO14/178910, WO14/178913, WO14/178941, WO14/179276, WO16/183445, WO17/062831, WO17/062825; for rice events LLRICE06, LLRICE601, LLRICE62 in WO00/026345, WO00/026356, WO00/026345; and for soybean events H7-1, MON89788, A2704-12, A5547-127, DP305423, DP356043, MON87701, MON87769, CV127, MON87705, DAS68416-4, MON87708, MON87712, SYHTOH2, DAS81419, DAS81419 x DAS44406-6, MON87751 in WO04/074492, WO06/130436, WO06/108674, WO06/108675, WO08/054747, WO08/002872, WO09/064652, WO09/102873, WO10/080829, WO10/037016, WO11/066384, WO11/034704, WO12/051199, WO12/082548, WO13/016527, WO13/016516, WO14/201235.

The use of spraying liquids on cultivated plants may result in effects which are specific to a cultivated plant comprising a certain transgene or event. These effects might involve changes in growth behavior or changed resistance to biotic or abiotic stress factors. Such effects may in particular comprise enhanced yield, enhanced resistance or tolerance to insects, nematodes, fungal, bacterial, mycoplasma, viral or viroid pathogens as well as early vigour, early or delayed ripening, cold or heat tolerance as well as changed amino acid or fatty acid spectrum or content.

The spraying liquids are particularly suitable for controlling the following causal agents of plant diseases:

Albugo spp. (white rust) on ornamentals, vegetables (e. g. A. candida) and sunflowers (e. g. A. tragopogonis); Alternaria spp. (Alternaria leaf spot) on vegetables (e.g. A. dauci or A. porri), oilseed rape (A. brassicicola or brassicae), sugar beets (A. tenuis), fruits (e.g. A. grandis), rice, soybeans, potatoes and tomatoes (e. g. A. solani, A. grandis or A. alternata), tomatoes (e. g. A. solani or A. alternata) and wheat (e.g. A. triticina); Aphanomyces spp. on sugar beets and vegetables; Ascochyta spp. on cereals and vegetables, e. g. A. tritici (anthracnose) on wheat and A. hordei on barley; Aureobasidium zeae (syn. Kapatiella zeae) on corn; Bipolaris and Drechslera spp. (teleomorph: Cochliobolus spp.), e. g. Southern leaf blight (D. maydis) or Northern leaf blight (B. zeicola) on corn, e. g. spot blotch (B. sorokiniana) on cereals and e. g. B. oryzae on rice and turfs; Blumeria (formerly Erysiphe) graminis (powdery mildew) on cereals (e. g. on wheat or barley); Botrytis cinerea (teleomorph: Botryotinia fuckeliana: grey mold) on fruits and berries (e. g. strawberries), vegetables (e. g. lettuce, carrots, celery and cabbages); B. squamosa or B. allii on onion family), oilseed rape, ornamentals (e.g. B eliptica), vines, forestry plants and wheat; Bremia lactucae (downy mildew) on lettuce; Ceratocystis (syn. Ophiostoma) spp. (rot or wilt) on broad-leaved trees and evergreens, e. g. C. ulmi (Dutch elm disease) on elms; Cercospora spp. (Cercospora leaf spots) on corn (e. g. Gray leaf spot: C. zeae-maydis), rice, sugar beets (e. g. C. beticola), sugar cane, vegetables, coffee, soybeans (e. g. C. sojina or C. kikuchii) and rice; Cladobotryum (syn. Dactylium) spp. (e.g. C. mycophilum (formerly Dactylium dendroides, teleomorph: Nectria albertinii, Nectria rosella syn. Hypomyces rosellus) on mushrooms; Cladosporium spp. on tomatoes (e. g. C. fulvum: leaf mold) and cereals, e. g. C. herbarum (black ear) on wheat; Claviceps purpurea (ergot) on cereals; Cochliobolus (anamorph: Helminthosporium of Bipolaris) spp. (leaf spots) on corn (C. carbonum), cereals (e. g. C. sativus, anamorph: B. sorokiniana) and rice (e. g. C. miyabeanus, anamorph: H. oryzae); Colletotrichum (teleomorph: Glomerella) spp. (anthracnose) on cotton (e. g. C. gossypii), corn (e. g. C. graminicola: Anthracnose stalk rot), soft fruits, potatoes (e. g. C. coccodes: black dot), beans (e. g. C. lindemuthianum), soybeans (e. g. C. truncatum or C. gloeosporioides), vegetables (e.g. C. lagenarium or C. capsici), fruits (e.g. C. acutatum), coffee (e.g. C. coffeanum or C. kahawae) and C. gloeosporioides on various crops; Corticium spp., e. g. C. sasakii (sheath blight) on rice; Corynespora cassiicola (leaf spots) on soybeans, cotton and ornamentals; Cycloconium spp., e. g. C. oleaginum on olive trees; Cylindrocarpon spp. (e. g. fruit tree canker or young vine decline, teleomorph: Nectria or Neonectria spp.) on fruit trees, vines (e. g. C. liriodendri, teleomorph: Neonectria liriodendri: Black Foot Disease) and ornamentals; Dematophora (teleomorph: Rosellinia) necatrix (root and stem rot) on soybeans; Diaporthe spp., e. g. D. phaseolorum (damping off) on soybeans; Drechslera (syn. Helminthosporium, teleomorph: Pyrenophora) spp. on corn, cereals, such as barley (e. g. D. teres, net blotch) and wheat (e. g. D. tritici-repentis: tan spot), rice and turf; Esca (dieback, apoplexy) on vines, caused by Formitiporia (syn. Phellinus) punctata, F. mediterranea, Phaeomoniella chlamydospora (formerly Phaeoacremonium chlamydosporum), Phaeoacremonium aleophilum and/or Botryosphaeria obtusa; Elsinoe spp. on pome fruits (E. pyri), soft fruits (E. veneta: anthracnose) and vines (E. ampelina: anthracnose); Entyloma oryzae (leaf smut) on rice; Epicoccum spp. (black mold) on wheat; Erysiphe spp. (powdery mildew) on sugar beets (E. betae), vegetables (e. g. E. pisi), such as cucurbits (e. g. E. cichoracearum), cabbages, oilseed rape (e. g. E. cruciferarum); Eutypalata (Eutypa canker or dieback, anamorph: Cytosporina lata, syn. Libertella blepharis) on fruit trees, vines and ornamental woods; Exserohilum (syn. Helminthosporium) spp. on corn (e. g. E. turcicum); Fusarium (teleomorph: Gibberella) spp. (wilt, root or stem rot) on various plants, such as F. graminearum or F. culmorum (root rot, scab or head blight) on cereals (e. g. wheat or barley), F. oxysporum on tomatoes, F. solani (f. sp. glycines now syn. F. virguliforme) and F. tucumaniae and F. brasiliense each causing sudden death syndrome on soybeans, and F. verticillioides on corn; Gaeumannomyces graminis (take-all) on cereals (e. g. wheat or barley) and corn; Gibberella spp. on cereals (e. g. G. zeae) and rice (e. g. G. fujikuroi: Bakanae disease); Glomerella cingulata on vines, pome fruits and other plants and G. gossypii on cotton; Grainstaining complex on rice; Guignardia bidwellii (black rot) on vines; Gymnosporangium spp. on rosaceous plants and junipers, e. g. G. sabinae (rust) on pears; Helminthosporium spp. (syn. Drechslera, teleomorph: Cochliobolus) on corn, cereals, potatoes and rice; Hemileia spp., e. g. H. vastatrix (coffee leaf rust) on coffee; Isariopsis clavispora (syn. Cladosporium vitis) on vines; Macrophomina phaseolina (syn. phaseoli) (root and stem rot) on soybeans and cotton; Microdochium (syn. Fusarium) nivale (pink snow mold) on cereals (e. g. wheat or barley); Microsphaera diffusa (powdery mildew) on soybeans; Monilinia spp., e. g. M. laxa, M. fructicola and M. fructigena (syn. Monilia spp.: bloom and twig blight, brown rot) on stone fruits and other rosaceous plants; Mycosphaerella spp. on cereals, bananas, soft fruits and ground nuts, such as e. g. M. graminicola (anamorph: Zymoseptoria tritici formerly Septoria tritici: Septoria blotch) on wheat or M. fijiensis (syn. Pseudocercospora fijiensis: black Sigatoka disease) and M. musicola on bananas, M. arachidicola (syn. M. arachidis or Cercospora arachidis), M. berkeleyi on peanuts, M. pisi on peas and M. brassiciola on brassicas; Peronospora spp. (downy mildew) on cabbage (e. g. P. brassicae), oilseed rape (e. g. P. parasitica), onions (e. g. P. destructor), tobacco (P. tabacina) and soybeans (e. g. P. manshurica); Phakopsora pachyrhizi and P. meibomiae (soybean rust) on soybeans; Phialophora spp. e. g. on vines (e. g. P. tracheiphila and P. tetraspora) and soybeans (e. g. P. gregata: stem rot); Phoma lingam (syn. Leptosphaeria biglobosa and L. maculans: root and stem rot) on oilseed rape and cabbage, P. betae (root rot, leaf spot and damping-off) on sugar beets and P. zeae-maydis (syn. Phyllostica zeae) on corn; Phomopsis spp. on sunflowers, vines (e. g. P. viticola: can and leaf spot) and soybeans (e. g. stem rot: P. phaseoli, teleomorph: Diaporthe phaseolorum); Physoderma maydis (brown spots) on corn; Phytophthora spp. (wilt, root, leaf, fruit and stem root) on various plants, such as paprika and cucurbits (e. g. P. capsici), soybeans (e. g. P. megasperma, syn. P. sojae), potatoes and tomatoes (e. g. P. infestans: late blight) and broad-leaved trees (e. g. P. ramorum: sudden oak death); Plasmodiophora brassicae (club root) on cabbage, oilseed rape, radish and other plants; Plasmopara spp., e. g. P. viticola (grapevine downy mildew) on vines and P. halstedii on sunflowers; Podosphaera spp. (powdery mildew) on rosaceous plants, hop, pome and soft fruits (e. g. P. leucotricha on apples) and curcurbits (P. xanthii); Polymyxa spp., e. g. on cereals, such as barley and wheat (P. graminis) and sugar beets (P. betae) and thereby transmitted viral diseases; Pseudocercosporella herpotrichoides (syn. Oculimacula yallundae, O. acuformis: eyespot, teleomorph: Tapesia yallundae) on cereals, e. g. wheat or barley; Pseudoperonospora (downy mildew) on various plants, e. g. P. cubensis on cucurbits or P. humili on hop; Pseudopezicula tracheiphila (red fire disease or, rotbrenner', anamorph: Phialophora) on vines; Puccinia spp. (rusts) on various plants, e. g. P. triticina (brown or leaf rust), P. striiformis (stripe or yellow rust), P. hordei (dwarf rust), P. graminis (stem or black rust) or P. recondita (brown or leaf rust) on cereals, such as e. g. wheat, barley or rye, P. kuehnii (orange rust) on sugar cane and P. asparagi on asparagus; Pyrenopeziza spp., e.g. P. brassicae on oilseed rape; Pyrenophora (anamorph: Drechslera) tritici-repentis (tan spot) on wheat or P. teres (net blotch) on barley; Pyricularia spp., e. g. P. oryzae (teleomorph: Magnaporthe grisea: rice blast) on rice and P. grisea on turf and cereals; Pythium spp. (damping-off) on turf, rice, corn, wheat, cotton, oilseed rape, sunflowers, soybeans, sugar beets, vegetables and various other plants (e. g. P. ultimum or P. aphanidermatum) and P. oligandrum on mushrooms; Ramularia spp., e. g. R. collo-cygni (Ramularia leaf spots, Physiological leaf spots) on barley, R. areola (teleomorph: Mycosphaerella areola) on cotton and R. beticola on sugar beets; Rhizoctonia spp. on cotton, rice, potatoes, turf, corn, oilseed rape, potatoes, sugar beets, vegetables and various other plants, e. g. R. solani (root and stem rot) on soybeans, R. solani (sheath blight) on rice or R. cerealis (Rhizoctonia spring blight) on wheat or barley; Rhizopus stolonifer (black mold, soft rot) on strawberries, carrots, cabbage, vines and tomatoes; Rhynchosporium secalis and R. commune (scald) on barley, rye and triticale; Sarocladium oryzae and S. attenuatum (sheath rot) on rice; Sclerotinia spp. (stem rot or white mold) on vegetables (S. minor and S. sclerotiorum) and field crops, such as oilseed rape, sunflowers (e. g. S. sclerotiorum) and soybeans, S. rolfsii (syn. Athelia rolfsii) on soybeans, peanut, vegetables, corn, cereals and ornamentals; Septoria spp. on various plants, e. g. S. glycines (brown spot) on soybeans, S. tritici (syn. Zymoseptoria tritici, Septoria blotch) on wheat and S. (syn. Stagonospora) nodorum (Stagonospora blotch) on cereals; Uncinula (syn. Erysiphe) necator (powdery mildew, anamorph: Oidium tuckeri) on vines; Setosphaeria spp. (leaf blight) on corn (e. g. S. turcicum, syn. Helminthosporium turcicum) and turf; Sphacelotheca spp. (smut) on corn, (e. g. S. reiliana, syn. Ustilago reiliana: head smut), sorghum und sugar cane; Sphaerotheca fuliginea (syn. Podosphaera xanthii: powdery mildew) on cucurbits; Spongospora subterranea (powdery scab) on potatoes and thereby transmitted viral diseases; Stagonospora spp. on cereals, e. g. S. nodorum (Stagonospora blotch, teleomorph: Leptosphaeria [syn. Phaeosphaeria] nodorum, syn. Septoria nodorum) on wheat; Synchytrium endobioticum on potatoes (potato wart disease); Taphrina spp., e. g. T. deformans (leaf curl disease) on peaches and T. pruni (plum pocket) on plums; Thielaviopsis spp. (black root rot) on tobacco, pome fruits, vegetables, soybeans and cotton, e. g. T. basicola (syn. Chalara elegans); Tilletia spp. (common bunt or stinking smut) on cereals, such as e. g. T. tritici (syn. T. caries, wheat bunt) and T. controversa (dwarf bunt) on wheat; Trichoderma harzianum on mushrooms; Typhula incarnata (grey snow mold) on barley or wheat; Urocystis spp., e. g. U. occulta (stem smut) on rye; Uromyces spp. (rust) on vegetables, such as beans (e. g. U. appendiculatus, syn. U. phaseoli), sugar beets (e. g. U. betae or U. beticola) and on pulses (e.g. U. vignae, U. pisi, U.viciae-fabae and U. fabae); Ustilago spp. (loose smut) on cereals (e. g. U. nuda and U. avaenae), corn (e. g. U. maydis: corn smut) and sugar cane; Venturia spp. (scab) on apples (e. g. V. inaequalis) and pears; and Verticillium spp. (wilt) on various plants, such as fruits and ornamentals, vines, soft fruits, vegetables and field crops, e. g. V. longisporum on oilseed rape, V. dahliae on strawberries, oilseed rape, potatoes and tomatoes, and V. fungicola on mushrooms; Zymoseptoria tritici on cereals.

The spraying liquids are particularly suitable for controlling the following causal agents of plant diseases: rusts on soybean and cereals (e.g. Phakopsora pachyrhizi and P. meibomiae on soy; Puccinia tritici and P. striiformis on wheat); molds on specialty crops, soybean, oil seed rape and sunflowers (e.g. Botrytis cinerea on strawberries and vines, Sclerotinia sclerotiorum, S. minor and S. rolfsii on oil seed rape, sunflowers and soybean); Fusarium diseases on cereals (e.g. Fusarium culmorum and F. graminearum on wheat); downy mildews on specialty crops (e.g. Plasmopara viticola on vines, Phytophthora infestans on potatoes); powdery mildews on specialty crops and cereals (e.g. Uncinula necator on vines, Erysiphe spp. on various specialty crops, Blumeria graminis on cereals); and leaf spots on cereals, soybean and corn (e.g. Septoria tritici and S. nodorum on cereals, S. glycines on soybean, Cercospora spp. on corn and soybean).

The spraying liquids may be used for improving the health of a plant. The invention also relates to a method for improving plant health by treating a plant, its propagation material, and/or the locus where the plant is growing or is to grow with an effective amount of compounds I and compositions thereof, respectively.

The term “plant health” is to be understood to denote a condition of the plant and/or its products which is determined by several indicators alone or in combination with each other, such as yield (e. g. increased biomass and/or increased content of valuable ingredients), plant vigor (e. g. improved plant growth and/or greener leaves (“greening effect”)), quality (e. g. improved content or composition of certain ingredients), and tolerance to abiotic and/or biotic stress. The above identified indicators for the health condition of a plant may be interdependent or may result from each other.

The invention is further illustrated by the following non-limiting embodiments.

Embodiment 1: Spraying liquid suitable for spraying plants, comprising:
a. 0.1%-v/v to 2%-v/v of at least one additive selected from the groups i. to x.:
   i. EO-PO block polymers having a HLB (hydrophilic-lipophilic-balance) value of 1, 2, 3, 4, 6, 8, 12, 13, 14, 15, 27 or 29 and an average molecular weight (AMW) between 1500 and 15000,
   ii. methyl esters of unsaturated or saturated C6 to C18 fatty acids,
   iii. ethylenediamine tetra-functional PO/EO block polymers with a molecular weight (AMW) between 2000 and 20000 s,
   iv. C8 to C14 alkyl polyglycosides,
   v. fatty acid polyethylene esters comprising C12 to C18 saturated or unsaturated fatty acids
   vi. ethoxylated triglycerides derived from plant oils,
   vii. alkoxylated alcohols, having from 2 to 80 oxyethylene units and from 2 to 40 oxypropylene units and C4 to C18 alcohols,
   viii. alkyl naphthalene sulfonates,
   ix. alkyl polyglycoside lignosulfonate blends,
   x. polyquaternium polymers,
b. 0.002%-v/v to 2.0%-v/v bacterial spores or vegetative cells,
c. optionally 0.001%-v/v to 10.0%-v/v of further components, and
d. up to 100%-v/v water.

### Embodiment 2

The spraying liquid of embodiment 1, wherein at least one additive is selected from the group comprising EO/PO/EO block polymers with an HLB value of 1, 2, 3, 12, 13, 14, 15, 27 or 29, PO/EO/PO block polymers with an HLB value of 4, 6, 8, 12 or 15, C6-C10 fatty acid methyl ester, C12-C18 fatty acid methyl ester, soya fatty acid methyl ester, oleic acid methyl ester, ethylenediamine tetra-functional PO/EO block polymers with an molecular weight of 3600, 4700 or 15000, C8-C10 alkylpolyglycosides, PEG 400 monolaurate, PEG 400 monooleate, PEG 400 dioleate, soybean oil POE 10, castor oil POE 16, castor oil POE 40, alkoxylated alcohol HLB 9 or 12, butyl alcohol POP 31 POE 31, dibutyl naphthalene sulfonate, or a lignosulfonate C8-C10 alkylpolyglycoside blend.

### Embodiment 3

The spraying liquid of embodiment 1 or 2, wherein at least one additive is selected from the group comprising EO/PO/EO block polymers with an HLB value of 2, 12, or 15, PO/EO/PO block polymer with an HLB value of 4, C6-C10 fatty acid methyl ester, C12C18 fatty acid methyl ester, soya fatty acid methyl ester, oleic acid methyl ester, ethylenediamine tetra-functional PO/EO block polymer with a molecular weight of 15000, C8-C10 alkylpolyglycosides, PEG 400 dioleate, soybean oil POE 10, alkoxylated alcohol with an HLB value of 12, or a dibutyl naphthalene sulfonate.

### Embodiment 4

The spraying liquid of any one of embodiments 1 to 3, wherein at least one additive is selected from the group comprising EO/PO/EO block polymers with an HLB value of 1, 2, 3, 12, 13, 14, 15, 27 or 29, and one or more additive is selected from EO/PO/EO block polymers with an HLB value of 1, 2, 3, 12, 13, 14, 15, 27 or 29, PO/EO/PO block polymers with an HLB value of 4, 6, 8, 12 or 15, C6-C10 fatty acid methyl ester, C12-C18 fatty acid methyl ester, soya fatty acid methyl ester, oleic acid methyl ester, ethylenediamine tetra-functional PO/EO block polymer with an molecular weight of 3600, 4700 or 15000, C8-C10 alkylpolyglycosides, PEG 400 monolaurate, PEG 400 monooleate, PEG 400 dioleate, soybean oil POE 10, castor oil POE 16, castor oil POE 40, alkoxylated alcohol HLB 9 or 12, butyl alcohol POP 31 POE 31, dibutyl naphthalene sulfonate, or a lignosulfonate C8-C10 alkylpolyglycoside blend, wherein the selected additives are not identical.

### Embodiment 5

The spraying liquid of any one of embodiments 1 to 4, wherein at least one additive is selected from the group comprising EO/PO/EO block polymers with an HLB value of 2 or 12, C6-C10 fatty acid methyl ester, soya fatty acid methyl ester, PEG 400 dioleate, soybean oil POE 10, alkoxylated alcohol HLB 12, dibutyl naphthalene sulfonate.

### Embodiment 6

The spraying liquid of any one of embodiments 1 to 5, wherein at least one additive is selected from the group comprising EO/PO/EO block polymers with an HLB value of 2 or 12, PEG 400 dioleate, soybean oil POE 10, alkoxylated alcohol with an HLB value of 12, or dibutyl naphthalene sulfonate.

### Embodiment 7

The spraying liquid of any one of embodiments 1 to 6, wherein the spraying liquid comprises a blend of additives comprising one or more additives selected from the group of ethoxylated triglycerides, preferably selected from ethoxylated soybean oil with POE 10, 30 42 or 60, ethoxylated castor oil with POE 2.5, 5, 7, 16, 18, 20, 25, 30, 33, 35, 36, 40, 44, 60 or 200, ethoxylated rapeseed oil with POE 30 and one or more additives selected from the group of a) to d):
a) EO-PO block polymers having a HLB (hydrophilic-lipophilic-balance) value of 1, 2, 3, 4, 6, 8, 12, 13, 14, 15, 27 or 29 and an average molecular weight between 1500 and 15000,
b) fatty acid polyethylene esters,
c) alkoxylated alcohols,
d) alkyl naphthalene sulfonates,
wherein the % v/v ratio of the additives of the two groups is between 10:1 and 1:1, and if one or more additives of the group of ethoxylated triglycerides and/or group a), b), c) or d) is present, the total amount of all additives per group is calculated to determine the ratio.

### Embodiment 8

The spraying liquid of any one of claims 1 to 7, wherein the spraying liquid comprises a blend of additives as defined in a. to g:
a. an additive blend comprising 1 to 99% v/v of at least one additive selected from: soybean oil POE 10, castor oil POE 16 and castor oil POE 40, preferably at least one or all is soybean oil POE 10, and 1 to 99% v/v of at least one additive selected from: PEG 400 monolaurate, PEG 400 monooleate and PEG 400 dioleate, preferably at least one or all is PEG 400 dioleate, wherein the % v/v of both components add up to 100%,
b. an additive blend comprising 1 to 98% v/v of at least one additive selected from: soybean oil POE 10, castor oil POE 16 and castor oil POE 40, preferably at least one or all is soybean oil POE 10, and 1 to 98 v/v of at least one additive selected from: PEG 400 monolaurate, PEG 400 monooleate and PEG 400 dioleate, preferably at least one or all is PEG 400 dioleate and 1 to 98% v/v of at least one additive selected from: alkoxylated alcohol having an HLB value of 9 or 12, preferably at least one or all has an HLB value of 12, wherein the % v/v of all components add up to 100%.,
c. an additive blend comprising 1 to 99% v/v of at least one additive selected from: soybean oil POE 10, castor oil POE 16 and castor oil POE 40, preferably at least one or all is soybean oil POE 10, and 1 to 99% v/v of dibutyl naphthalene sulfonate, preferably sodium dibutyl naphthalene sulfonate, wherein the % v/v of all components add up to 100%.,
d. an additive blend comprising 1 to 98% v/v of at least one additive selected from: soybean oil POE 10, castor oil POE 16 and castor oil POE 40, preferably at least one or all is soybean oil POE 10, and 1 to 98% v/v of at least one additive selected from: PEG 400 monolaurate, PEG 400 monooleate and PEG 400 dioleate, preferably at least one or all is PEG 400 dioleate, and 1 to 98% v/v of at least one additive selected from: EO/PO/EO block polymers with an HLB value of 1, 2, 3, 12, 13, 14, 15, 27 and 29, preferably at least one or all have an HLB value of 2 or 12, more preferred at least one or all have a HLB value of 2, wherein the % v/v of all components add up to 100%;
e. an additive blend comprising 1 to 95% v/v of at least one additive selected from: soybean oil POE 10, castor oil POE 16 and castor oil POE 40, preferably at least one or all is soybean oil POE 10, and 1 to 95% v/v of at least one additive selected from: PEG 400 monolaurate, PEG 400 monooleate and PEG 400 dioleate, preferably at least one or all is PEG 400 dioleate, and 1 to 95% v/v dibutyl naphthalene sulfonate, preferably sodium dibutyl naphthalene sulfonate, and 1 to 95% v/v of at least one additive selected from: alkoxylated alcohol having an HLB value of 9 or 12, preferably at least one or all has an HLB value of 12, and 1 to % v/v at least one selected from: EO/PO/EO block polymers with an HLB value of 1, 2, 3, 12, 13, 14, 15, 27 and 29, preferably at least one has an HLB value of 2, 12, or 15, more preferred at least one has an HLB value of 2, wherein the % v/v of all components add up to 100%,
f. an additive blend comprising 1 to 97% v/v of at least one additive selected from: soybean oil POE 10, castor oil POE 16 and castor oil POE 40, preferably at least one or all is soybean oil POE 10, and 1 to 97% v/v dibutyl naphthalene sulfonate, preferably sodium dibutyl naphthalene sulfonate, and 1 to 97% v/v an alkoxylated alcohol having an HLB value of 9 or 12, preferably at least one or all has an HLB value of 12, wherein the % v/v of all components add up to 100%;
g. an additive blend comprising 1 to 97% v/v of at least one additive selected from: EO/PO/EO block polymers with an HLB value of 1, 2, 3, 12, 13, 14, 15, 27 and 29, preferably at least one or all have an HLB value of 2, or 12, more preferred at least one or all have a HLB value of 2, and 1 to 97% v/v of at least one additive selected from: PEG 400 monolaurate, PEG 400 monooleate and PEG 400 dioleate, preferably at least one or all is PEG 400 dioleate, and 1 to 97% v/v of at least one additive selected from: soybean oil POE 10, castor oil POE 16 and castor oil POE 40, preferably at least one or all is soybean oil POE 10, wherein the % v/v of all components add up to 100%.

### Embodiment 9

The spraying liquid of any one of embodiments 1 to 8, wherein the additive is a blend comprising
a. 45 to 55% v/v of at least one additive selected from: soybean oil POE 10, castor oil POE 16 and castor oil POE 40, preferably at least one or all is soybean oil POE 10, and 45 to 55% v/v of at least one additive selected from: PEG 400 monolaurate, PEG 400 monooleate and PEG 400 dioleate, preferably at least one or all is PEG 400 dioleate, wherein the % v/v of both components add up to 100%, or
b. 40 to 75% v/v of at least one additive selected from: soybean oil POE 10, castor oil POE 16 and castor oil POE 40, preferably at least one or all is soybean oil POE 10, 15 to 40% v/v of at least one additive selected from: PEG 400 monolaurate, PEG 400 monooleate and PEG 400 dioleate, preferably at least one or all is PEG 400 dioleate, and 10 to 20% v/v of at least one additive selected from: alkoxylated alcohol having an HLB value of 9 or 12, preferably at least one or all has an HLB value of 12 wherein the % v/v of both components add up to 100%, or
c. 60 to 75% v/v of at least one additive selected from: soybean oil POE 10, castor oil POE 16 or castor oil POE 40, preferably a soybean oil POE 10, and 25 to 40% v/v dibutyl naphthalene sulfonate, preferably sodium dibutyl naphthalene sulfonate, wherein the % v/v of all components add up to 100%, or
d. 40 to 60% v/v of at least one additive selected from: soybean oil POE 10, castor oil POE 16 and castor oil POE 40, preferably at least one or all is soybean oil POE 10, and 15 to 40% v/v of at least one additive selected from: PEG 400 monolaurate, PEG 400 monooleate and PEG 400 dioleate, preferably at least one or all is PEG 400 dioleate, and 15 to 30% v/v of an EO/PO/EO block polymers with an HLB value of 12 and 15 to 30% v/v of an EO/PO/EO block polymers with an HLB value of 2, wherein the % v/v of all components add up to 100%, or
e. 20 to 78% v/v of at least one additive selected from: soybean oil POE 10, castor oil POE 16 and castor oil POE 40, preferably at least one or all is soybean oil POE 10, and 1 to 20% v/v at least one additive selected from: PEG 400 monolaurate, PEG 400 monooleate and PEG 400 dioleate, preferably at least one or all is PEG 400 dioleate, and 10 to 40% v/v dibutyl naphthalene sulfonate, preferably sodium dibutyl naphthalene sulfonate, and 10 to 40% of at least one additive selected from: alkoxylated alcohol having an HLB value of 9 or 12, preferably at least one or all has an HLB value of 12, and 1 to 20% v/v at least one selected from: EO/PO/EO block polymers with an HLB value of 1, 2, 3, 12, 13, 14, 15, 27 and 29, preferably at least one has an HLB value of 2, 12, or 15, more preferred at least one has an HLB value of 2, or
f. 30 to 70% v/v of at least one additive selected from: soybean oil POE 10, castor oil POE 16 and castor oil POE 40, preferably at least one or all is soybean oil POE 10, and 15 to 40% v/v dibutyl naphthalene sulfonate, preferably sodium dibutyl naphthalene sulfonate, and 15 to 40% v/v alkoxylated alcohol having an HLB value of 9 or 12, preferably at least one or all has an HLB value of 12, wherein the % v/v of all components add up to 100%, or
g. 10 to 30% v/v of at least one additive selected from: EO/PO/EO block polymers with an HLB value of 1, 2, 3, 12, 13, 14, 15, 27 and 29, preferably at least one or all have an HLB value of 2 or 12, more preferred at least one or all have a HLB value of 2, and 10 to 30% v/v of at least one additive selected from: PEG 400 monolaurate, PEG 400 monooleate and PEG 400 dioleate, preferably at least one or all is PEG 400 dioleate, and 40 to 80% v/v of at least one additive selected from: soybean oil POE 10, castor oil POE 16 and castor oil POE 40, preferably at least one or all is soybean oil POE 10, wherein the % v/v of all components add up to 100%.

### Embodiment 10

The spraying liquid of any one of claims 1 to 9, wherein the bacterial spores or vegetative cells are from the genus Bacillus, Lysinibacillus, Paenibacillus or Streptomyces.

### Embodiment 11

The spraying liquid of any one of embodiments 1 or 10, wherein the bacterial spores or vegetative cells are from the species Bacillus subtilis, Bacillus mycoides, Bacillus licheniformis, Bacillus firmus, Bacillus amyloliquefaciens, Bacillus velezenis, Bacillus mycoides, Bacillus simplex, Bacillus thuringiensis, Bacillus pumilus, Paenibacillus polymyxa, Paenibacillus peoriae, Paenibacillus kribbensis, Paenibacillus terrae or Streptomyces lydicus.

### Embodiment 12

The spraying liquid of any one of embodiments 1 to 11, wherein the bacterial spores or vegetative cells are from the species Bacillus subtilis, Bacillus amyloliquefaciens or Bacillus velezenis.

### Embodiment 13

The spraying liquid of any one of embodiments 1 to 9, wherein the bacterial spores or vegetative cells are from the genus Pseudomonas, Burkholderia, Paraburkholderia or Rhizobium.

### Embodiment 14

The spraying liquid of any one of embodiments 1 to 9 or embodiment 13, wherein the bacterial spores or vegetative cells are from the species Pseudomonas fluorescens, Pseudomonas chlororaphis, Burkholderia rinojensis, Paraburkholderia phytofirmans, Rhizobium leguminosarum, Rhizobium tropici, Rhizobium loti, Rhizobium trifolii, Rhizobium meliloti, Rhizobium fredii, Bradyrhizobium japonicum or Bradyrhizobium elkanii.

### Embodiment 15

The spraying liquid of any one of embodiments 1 to 9 or embodiment 12 or 13, wherein the bacterial vegetative cells are from the species Pseudomonas fluorescens.

### Embodiment 16

The spraying liquid of any one of embodiments 13 to 15, wherein at least one additive is selected from C6-C10 fatty acid methyl ester, C12-C18 fatty acid methyl ester, soya fatty acid methyl ester, oleic acid methyl ester, PEG 400 dioleate, soybean oil POE 10, and polyquaternium-7.

### Embodiment 17

The spraying liquid of any one of embodiments 12 to 15, wherein at least one additive is selected from C6-C10 fatty acid methyl ester, oleic acid methyl ester, PEG 400 dioleate and soybean oil POE 10.

### Embodiment 18.1

An additive blend comprising one or more additives selected from the group of ethoxylated triglycerides, preferably selected from ethoxylated soybean oil with POE 10, 30, 42 or 60 or mixtures thereof, ethoxylated castor oil with POE 2.5, 5, 7, 16, 18, 20, 25, 30, 33, 35, 36, 40, 44, 60 or 200, ethoxylated rapeseed oil with POE 30 and one or more additives selected from the group of A1) to D1):
A1) EO-PO block polymers having a HLB (hydrophilic-lipophilic-balance) value of 1, 2, 3, 4, 6, 8, 12, 13, 14, 15, 27 or 29 and an average molecular weight between 1500 and 15000,
B1) fatty acid polyethylene esters,
C1) alkoxylated alcohols,
D1) alkyl naphthalene sulfonates,
wherein the % v/v ratio of the additives of the two groups is between 10:1 and 1:1, and if one or more additives of the group of ethoxylated triglycerides and/or group A1), B1), C1) or D1) is present, the total amount of all additives per group is calculated to determine the ratio,

### Embodiment 18.2:

An additive blend comprising one or more additives selected from the group of EO-PO block polymers having a HLB (hydrophilic-lipophilic-balance) value of 1, 2, 3, 4, 6, 8, 12, 13, 14, 15, 27 or 29 and an average molecular weight between 1500 and 15000 and one or more additives selected from the groups of A2) to C2):
A2) fatty acid methyl esters preferably C6-C10 methyl caproate-caprylate-caprate, C8-C10 methyl caprylate-caprate, C8 methyl caprylate, C10 methyl caprate, C12 methyl laurate, methyl coconate, palm kernel methyl ester, C14 methyl myristate, C 16 methyl palmitate, C18 methyl stearate, methyl sunflowerate, palm oil methyl ester, methyl rapeate, methyl soyate and C18:1 methyl oleate more preferred C6-C10 fatty acid methyl ester, C12-C18 fatty acid methyl ester, soya fatty acid methyl ester, oleic acid methyl ester. More preferred are C6-C10 fatty acid methyl ester, soya fatty acid methyl ester, preferably a methyl soyate, and oleic acid methyl ester, preferably a C18:1 methyl oleate;
B2) ethylenediamine tetra-functional PO/EO block polymers, preferably ethylenediamine tetra-functional PO/EO block polymers with a molecular weight (AMW) between 2000 and 20000, more preferred between 3000 and 18000, even more preferred they have a molecular weight of 3600, 4700 or 15000 and preferably have an HLB of 3 or 24,
C2) fatty acid polyethylene esters, preferably PEG 400 monolaurate, PEG 400 monooleate, PEG 400 dioleate more preferred is PEG 400 dioleate.
wherein the % v/v ratio of the additives of the group of EO-PO block polymers and the one or more additives of the groups of A2), B2) or C2) is between 10:1 and 1:1, and if one or more additives of the group of EO-PO block polymers and/or group A2), B3), or C2) is present, the total amount of all additives per group is calculated to determine the ratio,

### Embodiment 18.3

An additive blend comprising one or more additives selected from the group of EO/PO/EO block polymers having a HLB (hydrophilic-lipophilic-balance) value of 1, 2, 3, 4, 6, 8, 12, 13, 14, 15, 27 or 29 and an average molecular weight between 1500 and 15000 and
one or more additives selected from the group of PO/EO/PO block polymers with an HLB value of 4, 6, 8, or 12 and an average molecular weight between 1500 and 4000,
wherein the % v/v ratio of the EO/PO/EO block polymers and the PO/EO/PO block polymers is between 100:1 and 1:1, and if one or more additives of the group of EO/PO/EO block polymers and/or the group of PO/EO/PO block polymers is present, the total amount of all additives per group is calculated to determine the ratio.

### Embodiment 19

An additive blend comprising a blend of additives as defined in any one of the alternatives a. to g.:
h. comprising 1 to 99% v/v of at least one additive selected from: soybean oil POE 10, castor oil POE 16 and castor oil POE 40, preferably at least one or all is soybean oil POE 10, and 1 to 99% v/v of at least one additive selected from: PEG 400 monolaurate, PEG 400 monooleate and PEG 400 dioleate, preferably at least one or all is PEG 400 dioleate,
i. comprising 1 to 98% v/v of at least one additive selected from: soybean oil POE 10, castor oil POE 16 and castor oil POE 40, preferably at least one or all is soybean oil POE 10, and 1 to 98 v/v of at least one additive selected from: PEG 400 monolaurate, PEG 400 monooleate and PEG 400 dioleate, preferably at least one or all is PEG 400 dioleate and 1 to 98% v/v of at least one additive selected from: alkoxylated alcohol having an HLB value of 9 or 12, preferably at least one or all has an HLB value of 12,
j. comprising 1 to 99% v/v of at least one additive selected from: soybean oil POE 10, castor oil POE 16 and castor oil POE 40, preferably at least one or all is soybean oil POE 10, and 1 to 99% v/v of dibutyl naphthalene sulfonate, preferably sodium dibutyl naphthalene sulfonate,
k. comprising 1 to 98% v/v of at least one additive selected from: soybean oil POE 10, castor oil POE 16 and castor oil POE 40, preferably at least one or all is soybean oil POE 10, and 1 to 98% v/v of at least one additive selected from: PEG 400 monolaurate, PEG 400 monooleate and PEG 400 dioleate, preferably at least one or all is PEG 400 dioleate, and 1 to 98% v/v of at least one additive selected from: EO/PO/EO block polymers with an HLB value of 1, 2, 3, 12, 13, 14, 15, 27 and 29, preferably at least one or all have an HLB value of 2 or 12, more preferred at least one or all have a HLB value of 2,
l. comprising 1 to 95% v/v of at least one additive selected from: soybean oil POE 10, castor oil POE 16 and castor oil POE 40, preferably at least one or all is soybean oil POE 10, and 1 to 95% v/v of at least one additive selected from: PEG 400 monolaurate, PEG 400 monooleate and PEG 400 dioleate, preferably at least one or all is PEG 400 dioleate, and 1 to 95% v/v dibutyl naphthalene sulfonate, preferably sodium dibutyl naphthalene sulfonate, and 1 to 95% v/v of at least one additive selected from: alkoxylated alcohol having an HLB value of 9 or 12, preferably at least one or all has an HLB value of 12, and 1 to % v/v at least one selected from: EO/PO/EO block polymers with an HLB value of 1, 2, 3, 12, 13, 14, 15, 27 and 29, preferably at least one has an HLB value of 2, 12, or 15, more preferred at least one has an HLB value of 2,
m. comprising 1 to 97% v/v of at least one additive selected from: soybean oil POE 10, castor oil POE 16 and castor oil POE 40, preferably at least one or all is soybean oil POE 10, and 1 to 97% v/v dibutyl naphthalene sulfonate, preferably sodium dibutyl naphthalene sulfonate, and 1 to 97% v/v an alkoxylated alcohol having an HLB value of 9 or 12, preferably at least one or all has an HLB value of 12,
n. comprising 1 to 97% v/v of at least one additive selected from: EO/PO/EO block polymers with an HLB value of 1, 2, 3, 12, 13, 14, 15, 27 and 29, preferably at least one or all have an HLB value of 2, or 12, more preferred at least one or all have a HLB value of 2, and 1 to 97% v/v of at least one additive selected from: PEG 400 monolaurate, PEG 400 monooleate and PEG 400 dioleate, preferably at least one or all is PEG 400 dioleate, and 1 to 97% v/v of at least one additive selected from: soybean oil POE 10, castor oil POE 16 and castor oil POE 40, preferably at least one or all is soybean oil POE 10,
   wherein the % v/v of all components of additive blends a. to g. and optionally water add up to 100%.

### Embodiment 20

An additive blend of embodiment 19, comprising a blend of additives as defined in any one of the alternatives a. to g.:
a. 45 to 55% v/v of at least one additive selected from: soybean oil POE 10, castor oil POE 16 and castor oil POE 40, preferably at least one or all is soybean oil POE 10, and 45 to 55% v/v of at least one additive selected from: PEG 400 monolaurate, PEG 400 monooleate and PEG 400 dioleate, preferably at least one or all is PEG 400 dioleate,
b. 40 to 75% v/v of at least one additive selected from: soybean oil POE 10, castor oil POE 16 and castor oil POE 40, preferably at least one or all is soybean oil POE 10, 15 to 40% v/v of at least one additive selected from: PEG 400 monolaurate, PEG 400 monooleate and PEG 400 dioleate, preferably at least one or all is PEG 400 dioleate, and 10 to 20% v/v of at least one additive selected from: alkoxylated alcohol having an HLB value of 9 or 12, preferably at least one or all has an HLB value of 12,
c. 60 to 75% v/v of at least one additive selected from: soybean oil POE 10, castor oil POE 16 or castor oil POE 40, preferably a soybean oil POE 10, and 25 to 40% v/v dibutyl naphthalene sulfonate, preferably sodium dibutyl naphthalene sulfonate,
d. 40 to 60% v/v of at least one additive selected from: soybean oil POE 10, castor oil POE 16 and castor oil POE 40, preferably at least one or all is soybean oil POE 10, and 15 to 40% v/v of at least one additive selected from: PEG 400 monolaurate, PEG 400 monooleate and PEG 400 dioleate, preferably at least one or all is PEG 400 dioleate, and 15 to 30% v/v of an EO/PO/EO block polymers with an HLB value of 12 and 15 to 30% v/v of an EO/PO/EO block polymers with an HLB value of 2,
e. 20 to 78% v/v of at least one additive selected from: soybean oil POE 10, castor oil POE 16 and castor oil POE 40, preferably at least one or all is soybean oil POE 10, and 1 to 20% v/v at least one additive selected from: PEG 400 monolaurate, PEG 400 monooleate and PEG 400 dioleate, preferably at least one or all is PEG 400 dioleate, and 10 to 40% v/v dibutyl naphthalene sulfonate, preferably sodium dibutyl naphthalene sulfonate, and 10 to 40% of at least one additive selected from: alkoxylated alcohol having an HLB value of 9 or 12, preferably at least one or all has an HLB value of 12, and 1 to 20% v/v at least one selected from: EO/PO/EO block polymers with an HLB value of 1, 2, 3, 12, 13, 14, 15, 27 and 29, preferably at least one has an HLB value of 2, 12, or 15, more preferred at least one has an HLB value of 2,
f. 30 to 70% v/v of at least one additive selected from: soybean oil POE 10, castor oil POE 16 and castor oil POE 40, preferably at least one or all is soybean oil POE 10, and 15 to 40% v/v dibutyl naphthalene sulfonate, preferably sodium dibutyl naphthalene sulfonate, and 15 to 40% v/v alkoxylated alcohol having an HLB value of 9 or 12, preferably at least one or all has an HLB value of 12,
g. 40 to 70% v/v of at least one additive selected from: soybean oil POE 10, castor oil POE 16 or castor oil POE 40, preferably a soybean oil POE 10, and 10 to 50% v/v dibutyl naphthalene sulfonate, preferably sodium dibutyl naphthalene sulfonate,
   wherein the % v/v of all components of additive blends a. to g. and optionally water add up to 100%.

### Embodiment 21

Kit of at least two parts to prepare a spraying liquid suitable for spraying plants or for preparing a seed treatment composition for treating seeds of plants, wherein the bacterial spores or vegetative cells of embodiments 1 to 17 are provided in a first concentrated form and at least one of the additive blends listed in embodiments 18 to 20 is provided in at least one further concentrated form and wherein the relative amounts of the bacterial spores or vegetative cells and the relative amounts of the additive in the kit are adapted to provide the amounts described in embodiment 1.

### Embodiment 22

Kit of at least two parts according to embodiment 21, wherein the spraying liquid for spraying plants or for preparing a seed treatment composition for treating seeds of plants is prepared to be used in a method to control phytopathogenic fungi or phytopathogenic bacteria.

### Embodiment 23

Method to control phytopathogenic fungi or phytopathogenic bacteria wherein an additive blend as described in any one of embodiments 18 to 20 or a kit of at least two parts as described in embodiments 21 or 22 is used to prepare a spraying liquid for spraying plants or for preparing a seed treatment composition for treating seeds and the spraying liquid for spraying plants is sprayed on plants or the plant seeds are treated with the seed treatment composition for treating seeds.

### Embodiment 24

Method to control phytopathogenic fungi or phytopathogenic bacteria according to embodiment 23, wherein the plants are selected from: potatoes, sugar beets, tobacco, wheat, rye, barley, oats, rice, corn, cotton, soybeans, oilseed rape, legumes, sunflowers, coffee or sugar cane; fruits; vines; table grapes, grapes for wine making or table grapes, ornamentals; or vegetables, such as cucumbers, tomatoes, pepper, beans or squashes.

### Embodiment 25

Method to control phytopathogenic fungi or phytopathogenic bacteria according to embodiments 23 or 24, wherein the plants are selected from the group comprising corn, small-grain cereals, strawberry, apple, pumpkin, cucumber, grape, bean, tomato.

### Embodiment 26

Method to control phytopathogenic fungi according to any one of embodiments 23 to 25, wherein the fungi are selected from: basidiomycetes, ascomycetes, deuteromycetes or oomycetes.

### Embodiment 27

Method to control phytopathogenic fungi according to any one of embodiments 23 to 26, wherein the fungi are selected from the group comprising the genera Pythium, Plasmopara, Phytophthora, Rhizoctonia, Botrytis, Venturia, Erysiphe, Setosphaeria and Puccinia.

### Embodiment 28

Method to control phytopathogenic bacteria according to embodiments 23 or 24, wherein the bacteria are selected from the group comprising the genera Pseudomonas.

### Embodiment 29

Use of an additive blend as described in any one of embodiments 18 to 20 to enhance the pesticidal effectiveness of a pesticidal microorganism in a method to control phytopathogenic fungi or phytopathogenic bacteria as described in any one of embodiments 23 to 27.

### Embodiment 30

Use of an additive blend as described in any one of embodiments 18 to 20 to prepare a spraying liquid as described in any one of embodiments 1 to 17.

### Embodiment 31

Use of a spraying liquid as described in any one of embodiments 1 to 17 in a method as described in any one of embodiments 23 to 27.

### Embodiment 32

Use of a kit of at least two parts as described in any one of embodiments 21 or 22 to prepare a spraying liquid as described in any one of embodiments 1 to 17.

Further non-limiting embodiments listing preferred ratios of additives and additive blends to the amount of pesticidal microorganisms are described in the examples.

### Examples

### Example 1: Test for Additive Compatibility With Microbial Growth

To determine the influence of additives on microbial growth (i.e. Compatibility), each target organism was exposed to the additives in the following general steps:
1. A buffered spore/cell solution is prepared
2. A 10% stock solution of additive is diluted to desired concentrations and prepared as samples with buffered spores (test) and without spores (control)
3. After spores are exposed to additive solutions for a time period, these solutions are diluted into tryptic soy broth (nutrient) and allowed to incubate at 30 C for 24-48 hours.

4. The Optical Density (OD) of five replicate samples is measured every 30 minutes and graphed to determine the slope of the exponential growth phase of the organism's growth curve.

If the micro-organism's growth slope showed no significant difference, the conclusion was that the combination of micro-organism and additive was NEUTRAL. If there was a reduction of micro-organism growth slope, the conclusion was that the combination was SUPPRESSIVE. If there was a significant increase of micro-organism growth slope (i.e. a rate increase>1.6X), the conclusion was that the combination was ENHANCING.

The testing was performed with both living cell cultures obtained from the American Type Culture Collection (ATCC) and with commercial products sold for fungicide applications:
Bacillus subtilis QST 713 (Agraquest/Bayer, ATCC: 55614);
Bacillus firmus I-1582 (Bayer, ATCC: 8247);
Pseudomonas fluorescens A506 (NuFarm, ATCC: 31948);
Serifel® Biofungicide (Bacillus amyloliquefaciens strain MBI 600, sold by BASF Corporation); BlightBan® A506 Biofungicide (Pseudomonas fluorescens strain A506, sold by NuFarm Americas Inc.);
Subtilex® NG Biofungicide (Bacillus subtilis strain MBI 600, sold by BASF Corporation).

The following is a specific protocol as carried out for one organism.

All surfaces, buffer solutions, growth media and containers must be sterile to avoid contamination. A spore solution of the Bacillus subtilis (2-5×10¹⁰ CFU/g) was prepared in Phosphate Buffered Saline (PBS)+0.1% Tween 80 by first adding 1 g of dry spore concentrate to a total volume of 10 ml 1×PBS+0.1% Tween 80 in a sterile screw capped vial to yield a stock solution that was 2-5×10⁹ CFU/ml. This solution was further diluted with 1×PBS+0.1% Tween 80 to make a spore stock solution that is 2-5×10⁷ CFU/ml. A 10% stock solution of an additive to test is first prepared in 1×PBS+0.1% Tween 80 to a total volume of 50 ml in a sterile 50 ml screw capped tube. The stock solution is used to prepare two sets of additive dilutions in sterile 15 ml tubes with caps as defined in the table below. One set will be used as the additive+spore samples and the other set will be the additive control (no spores).

| | | | | Volume Spore | Volume |
|---|---|---|---|---|---|
| Additive | | Volume 10% | Volume of | Stock solution 2 − | 1 × PBS + |
| Test | Additive | Additive stock | 1 × PBS + 0.1% | 5 × 10⁷ CFU/ml), | 0.1% Tween |
| Solutions | Conc, % | solution, ml | Tween 80, ml | ml | 80, ml |
| Additive + | 5 | 5 | 4 | 1 | 0 |
| Spore | | | | | |
| Additive + | 4 | 4 | 5 | 1 | 0 |
| Spore | | | | | |
| Additive + | 3 | 3 | 6 | 1 | 0 |
| Spore | | | | | |
| Additive + | 2 | 2 | 7 | 1 | 0 |
| Spore | | | | | |
| Additive + | 1 | 1 | 8 | 1 | 0 |
| Spore | | | | | |
| Additive + | 0.5 | 0.5 | 8.5 | 1 | 0 |
| Spore | | | | | |
| Additive + | 0.1 | 0.1 | 8.9 | 1 | 0 |
| Spore | | | | | |
| Additive + | 0.01 | 0.01 | 8.99 | 1 | 0 |
| Spore | | | | | |
| Additive + | 0 | 0 | 9 | 1 | 0 |
| Spore | | | | | |
| Additive | 5 | 5 | 4 | 0 | 1 |
| Control | | | | | |
| Additive | 4 | 4 | 5 | 0 | 1 |
| Control | | | | | |
| Additive | 3 | 3 | 6 | 0 | 1 |
| Control | | | | | |
| Additive | 2 | 2 | 7 | 0 | 1 |
| Control | | | | | |
| Additive | 1 | 1 | 8 | 0 | 1 |
| Control | | | | | |
| Additive | 0.5 | 0.5 | 8.5 | 0 | 1 |
| Control | | | | | |
| Additive | 0.1 | 0.1 | 8.9 | 0 | 1 |
| Control | | | | | |
| Additive | 0.01 | 0.01 | 8.99 | 0 | 1 |
| Control | | | | | |
| Additive | 0 | 0 | 9 | 0 | 1 |
| Control | | | | | |

After these two sets have been prepared, they are placed on an orbital shaker set at 225-300 rpm and held at 16° C. for 4 hours.

A sterile 96 well plate is prepared as outlined below to achieve serial dilutions of the test solutions. This plate is referred to as the Serial Dilution Plate.

| Plate | Additive + | 5% | | 4% | | 3% | | 2% | | 1% | | 0.5% | | 0.1% | | 0.01% | | 0% | |
|---|---|---|---|---|---|---|---|---|---|---|---|---|---|---|---|---|---|---|---|
| Row | Spore | Additive | | Additive | | Additive | | Additive | | Additive | | Additive | | Additive | | Additive | | Additive | |
| A | | 300 | μl | 300 | μl | 300 | μl | 300 | μl | 300 | μl | 300 | μl | 300 | μl | 300 | μl | 300 | μl |
| B | | 30 | μl | 30 | μl | 30 | μl | 30 | μl | 30 | μl | 30 | μl | 30 | μl | 30 | μl | 30 | μl |
| C | | 30 | μl | 30 | μl | 30 | μl | 30 | μl | 30 | μl | 30 | μl | 30 | μl | 30 | μl | 30 | μl |
| | Additive | 5% | | 4% | | 3% | | 2% | | 1% | | 0.5% | | 0.1% | | 0.01% | | 0% | |
| | Control | Additive | | Additive | | Additive | | Additive | | Additive | | Additive | | Additive | | Additive | | Additive | |
| D | | 300 | μl | 300 | μl | 300 | μl | 300 | μl | 300 | μl | 300 | μl | 300 | μl | 300 | μl | 300 | μl |
| E | | 30 | μl | 30 | μl | 30 | μl | 30 | μl | 30 | μl | 30 | μl | 30 | μl | 30 | μl | 30 | μl |
| F | | 30 | μl | 30 | μl | 30 | μl | 30 | μl | 30 | μl | 30 | μl | 30 | μl | 30 | μl | 30 | μl |

In Row A, 300 μl of each Additive Test Solution (with spores) corresponding to the additive concentrate is added into the well. To Rows B, C, E and F, 270 μl of sterile tryptic soy broth is added. 30 μl from each well of Row A is pipetted into the corresponding well of Row B to bring the total volume in the wells of Row B to 300 μl. The 96 well plate is shaken after every addition to a row before proceeding. 30 μl from each well of Row B is pipetted into the corresponding well of Row C to bring the total volume of the wells of Row C to 300 μl.

In Row D, 300 μl of each Additive Test Solution labeled Additive Control corresponding to the additive concentration in added into the well. 30 μl of each well of Row D is pipetted into the corresponding well of Row E to bring the total volume in the wells of Row E to 300 μl. 30 μl of each well of Row E is pipetted into the corresponding well of Row F to bring the total volume of the wells of Row F to 300 μl.

A Growth Results 96 well plate is prepared by add 270 μl of sterile tryptic soy broth to each test well. To each well in the first 4 rows of the 96 well plate, 30 μl of Row C of the Serial Dilution Plate is added. These four rows are replicates of the Additive Test Solution with Spores. To each well in the final 4 rows (E-H) of the 96 well plate, 30 μl of Row F of the Serial Dilution Plate is added.

| Plate | | 5% | 4% | 3% | 2% | 1% | 0.5% | 0.1% | 0.01% | 0% |
|---|---|---|---|---|---|---|---|---|---|---|
| Row | Replicate | Additive | Additive | Additive | Additive | Additive | Additive | Additive | Additive | Additive |
| A | Additive + | 30 μl | 30 μl | 30 μl | 30 μl | 30 μl | 30 μl | 30 μl | 30 μl | 30 μl |
| | Spore 1 | | | | | | | | | |
| B | Additive + | 30 μl | 30 μl | 30 μl | 30 μl | 30 μl | 30 μl | 30 μl | 30 μl | 30 μl |
| | Spore 2 | | | | | | | | | |
| C | Additive + | 30 μl | 30 μl | 30 μl | 30 μl | 30 μl | 30 μl | 30 μl | 30 μl | 30 μl |
| | Spore 3 | | | | | | | | | |
| D | Additive + | 30 μl | 30 μl | 30 μl | 30 μl | 30 μl | 30 μl | 30 μl | 30 μl | 30 μl |
| | Spore 4 | | | | | | | | | |
| E | Additive 1 | 30 μl | 30 μl | 30 μl | 30 μl | 30 μl | 30 μl | 30 μl | 30 μl | 30 μl |
| F | Additive 2 | 30 μl | 30 μl | 30 μl | 30 μl | 30 μl | 30 μl | 30 μl | 30 μl | 30 μl |
| G | Additive 3 | 30 μl | 30 μl | 30 μl | 30 μl | 30 μl | 30 μl | 30 μl | 30 μl | 30 μl |
| H | Additive 4 | 30 μl | 30 μl | 30 μl | 30 μl | 30 μl | 30 μl | 30 μl | 30 μl | 30 μl |

When the setup of the Results Plate is completed, it is incubated at 30° C. for 24 hours without shaking. The optical density (OD) at 620 nm of each well is recorded every 30 minutes using a 96 well plate reader.

The resulting data is then analysed by first taking the mean OD620 nm for each time point taken at each concentration for both the “additive+spore” and “additive blank”. The mean OD620 nm of the “additive blank” is subtracted from the mean OD620 nm “additive+spore” to get a true average growth. Once all true means are found, a linear trendline is created using time versus true mean absorbance at 620 nm. The rate of growth is obtained from the slope using Y=M(x)+B. The slope represents the rate of growth. The rate of growth of the concentrations of additive with the spores is compared to the 0% (untreated) sample slope. The percentage growth for each concentration of additive spore mixture can be calculated with the following equation.

((Mean Slope of x Concentration)−(Mean Slope of Untreated Sample))/Mean Slope of Untreated Sample)*100=Change in Growth Slope (%)

The following table summarizes the results of these laboratory evaluations, as represented in this application. N represents a Neutral result; S represents a Suppressive result and E represents an Enhancing result. Not all additives were tested with all organisms.

TABLE 1

| | | | Pseudomonas | Bacillus | Bacillus | Bacillus |
|---|---|---|---|---|---|---|
| Group | Additive Name | | fluorescens | subtilis | firmus | amyloliquefaciens |
| i-1 | EO/PO/EO Block Polymer HLB 12 | nonionic | — | E | E | E |
| i-2 | EO/PO/EO Block Polymer HLB 3 | nonionic | N | N | N | N |
| i-3 | EO/PO/EO Block Polymer HLB 15 | nonionic | N | N | N | N |
| i-4 | EO/PO/EO Block Polymer HLB 29 | nonionic | S | N | — | N |
| i-5 | EO/PO/EO Block Polymer HLB 2 | nonionic | N | E | E | E |
| i-6 | EO/PO/EO Block Polymer HLB 14 | nonionic | — | N | N | N |
| i-7 | EO/PO/EO Block Polymer HLB 1 | nonionic | — | N | N | N |
| i-8 | EO/PO/EO Block Polymer HLB 13 | nonionic | N | N | N | N |
| i-9 | EO/PO/EO Block Polymer HLB 27 | nonionic | N | N | — | N |
| i-10 | PO/EO/PO Block Polymer HLB 15 | nonionic | — | N | — | N |
| i-11 | PO/EO/PO Block Polymer HLB 6 | nonionic | — | N | — | N |
| i-12 | PO/EO/PO Block Polymer HLB 12 | nonionic | — | N | — | N |
| i-13 | PO/EO/PO Block Polymer HLB 4 | nonionic | S | N | N | N |
| i-14 | PO/EO/PO Block Polymer HLB 8 | nonionic | — | N | — | N |
| ii-1 | C6-C10 fatty acid methyl ester | solvent | N | N | N | N |
| ii-2 | C12-C18 fatty acid methyl ester | solvent | N | N | S | N |
| ii-3 | Soya fatty acid methyl ester | solvent | N | N | N | E |
| ii-4 | Oleic acid methyl ester | solvent | N | N | N | E |
| iii-1 | Ethylenediamine Tetra- | nonionic | N | N | N | N |
| | functional PO/EO Block | | | | | |
| | Polymer mol wt. 3600 | | | | | |
| iii-2 | Ethylenediamine Tetra- | nonionic | N | N | N | N |
| | functional PO/EO Block | | | | | |
| | Polymer mol wt. 4700 | | | | | |
| iii-3 | Ethylenediamine Tetra- | nonionic | N | N | N | — |
| | functional PO/EO Block | | | | | |
| | Polymer mol wt. 15000 | | | | | |
| iv-1 | C8-C10 alkylpolyglycoside | nonionic | N | N | N | N |
| iv-2 | C12-C16 alkylpolyglycoside | nonionic | N | S | S | S |
| v-1 | PEG 400 monolaurate | nonionic | S | N | — | N |
| v-2 | PEG 400 monooleate | nonionic | S | N | — | N |
| v-3 | PEG 400 dioleate | nonionic | E | E | E | E |
| vi-1 | Soybean Oil POE 10 | nonionic | E | N | S | N |
| vi-2 | Soybean Oil POE 30 | nonionic | N | S | N | S |
| vi-3 | Castor Oil POE 16 | nonionic | N | N | N | N |
| vi-4 | Castor Oil POE 40 | nonionic | — | N | N | N |
| vii-1 | Alkoxylated alcohol HLB 2.8 | nonionic | — | S | S | S |
| vii-2 | Alkoxylated alcohol HLB 10.6 | nonionic | — | S | S | S |
| vii-3 | Alkoxylated alcohol HLB 8 | nonionic | — | S | S | S |
| vii-4 | Alkoxylated alcohol HLB 12 | nonionic | — | E | — | E |
| vii-5 | Alkoxylated alcohol HLB 3.4 | nonionic | — | S | — | S |
| vii-6 | Alkoxylated alcohol HLB 9 | nonionic | — | N | — | N |
| vii-7 | Butyl alcohol POP 31 POE 31 | nonionic | N | N | N | N |
| viii-1 | Dibutyl Naphthalene | anionic | — | E | — | E |
| | Sulfonate, Sodium | | | | | |
| ix-1 | Lignosulfonate C8-C10 | anionic | — | N | — | N |
| | alkylpolyglycoside blend | | | | | |
| x-1 | Polyquaternium-7 | cationic | E | E | E | — |
| xi-1 | Tridecyl alcohol POE 3 | nonionic | S | S | S | S |
| xi-2 | Tridecyl alcohol POE 9 | nonionic | S | S | S | S |
| xi-3 | 2-propylheptanol POE 5 | nonionic | S | S | S | S |
| xi-4 | 2-propylheptanol Alkoxylate | nonionic | S | S | S | S |
| | with 5 moles EO | | | | | |
| xi-5 | 2-propylheptanol Alkoxylate | nonionic | S | S | S | S |
| | with 7 moles EO | | | | | |
| xii-1 | Sorbitan monooleate | nonionic | E | S | S | S |
| xii-2 | Sorbitan monolaurate POE 20 | nonionic | S | S | S | S |
| xii-3 | Sorbitan monooleate POE 20 | nonionic | N | N | S | N |
| xii-4 | Sorbitan trioleate POE 20 | nonionic | S | N | S | S |
| xiii-1 | Tallow amine POE15 | nonionic | E | S | S | S |
| xiii-2 | Tallow amine POE20 | nonionic | — | S | S | S |
| xiv-1 | Polyacrylic acid, Sodium | anionic | S | N | N | N |
| xv-1 | Dimethyl lactamide | solvent | N | S | N | S |
| xv-2 | C8-C10 dimethyl amide | solvent | S | S | S | S |
| xv-3 | C12-C14 dimethyl amide | solvent | — | S | S | S |
| xvi-1 | C8-C10 fatty alcohol | solvent | S | S | S | S |
| xvi-2 | Olleyl-Cetyl alcohol (80% Oleyl) | solvent | N | N | S | N |
| xvi-3 | Olleyl-Cetyl alcohol (90% Oleyl) | solvent | S | S | S | S |
| xvii-1 | Sodium Lauryl ether sulfate | anionic | S | S | S | S |
| xvii-2 | Sodium dodecylbenzene sulfonate | anionic | S | S | S | S |
| xvii-3 | Calcium dodecylbenzene sulfonate | anionic | N | N | S | — |
| xviii-1 | Magnesium Oleate | anionic | S | S | S | — |
| xviii-2 | Sodium Tallowate | anionic | S | — | — | — |
| xix-1 | Tridecyl alcohol POE 10 | anionic | S | S | S | S |
| | phosphate ester | | | | | |
| xix-2 | Phosphate Ester of | anionic | S | S | S | S |
| | Ethoxylated Alcohol | | | | | |
| xix-3 | Isodecyl alcohol POE6 | anionic | S | S | S | S |
| | phosphate ester | | | | | |

### Example 2

Eight solutions were prepared as described in Table 2a. These solutions were sprayed on first year grape vines showing no signs of Downy Mildew (Plasmopara viticola). The bio-fungicide in the trial was Serifel Biofungicide (Bacillus amyloliquefaciens sold by BASF Corporation). Six of the solutions included Additive Blend 1 (a 50:50 blend of POE 10 soybean oil and PEG 400 dioleate). Forum® Fungicide (dimethomorph sold by BASF Corporation) was sprayed as the synthetic benchmark comparison.

TABLE 2a

| Sample Name | “0” | “0.25” | “0.5” | “1.0” | Forum | “A” | “B” | “C” |
|---|---|---|---|---|---|---|---|---|
| Serifel ® | 0.38 g | 0.38 g | 0.38 g | 0.38 g | — | — | — | — |
| Biofungicide | | | | | | | | |
| Additive Blend 1 | — | 2.39 g | 4.78 g | 9.56 g | — | 2.39 g | 4.78 g | 9.56 g |
| Forum ® Fungicide | — | — | — | — | 0.54 g | — | — | — |
| Water (grams) | 999.62 | 997.23 | 994.84 | 990.06 | 999.46 | 997.61 | 995.22 | 990.44 |

Four applications were made over a month to the same grape vines. At the end of the month, the infection rates of Downy Mildew were evaluated by comparing to untreated grape vines. Table 2b demonstrates the improved control of Downy Mildew (lower %-Infection) when Additive Blend 1 was added to Serifel® Biofungicide spray solutions as well as the relative rate effects.

TABLE 2b

| Sample | Untreated | “0” | “0.25” | “0.5” | “1.0” | “Forum” | “A” | “B” | “C” |
|---|---|---|---|---|---|---|---|---|---|
| %-Infection | 12.2 | 8.3 | 6.8 | 2.8 | 1.3 | 1.6 | 4.0 | 2.75 | 3.5 |
| Downy Mildew | | | | | | | | | |
| Std Dev | 7.4 | 3.56 | 3.95 | 2.45 | 2.5 | 1.25 | 4.2 | 2.06 | 3.32 |
| Number of | 0 | 2.0E10 | 2.0E10 | 2.0E10 | 2.0E10 | 0 | 0 | 0 | 0 |
| CFU applied | | | | | | | | | |
| Ratio | 0:0 | 1:0 | 1:6.37 | 1:12.75 | 1:25.5 | 0:0 | 0:6.37 | 0:12.75 | 0:25.5 |
| Serifel:Additive | | | | | | | | | |

### Example 3

Eight solutions were prepared as described in Table 3a. These solutions were sprayed on spinach seedlings showing no signs of Downy Mildew (Peronospora effusa). The bio-fungicide in the trial was Serifel® Biofungicide (Bacillus amyloliquefaciens sold by BASF Corporation). Three of the solutions included Additive Blend 6 (a 40:16:20:24 blend of POE 10 Soybean Oil, Di-isobutyl Naphthalene Sulfonate-Sodium salt, an alkoxylated alcohol having an HLB value of 12 and water). Ridomil® Gold Copper Fungicide (mefenoxam and copper hydroxide sold by Syngenta Crop Protection), Quadris® Fungicide (azoxystrobin sold by Syngenta Crop Protection), Actigard® Fungicide (acibenzolar-S-methyl sold by Syngenta Crop Protection), Orondis® Ultra Fungicide (mandipropamid and oxathiapiprolin sold by Syngenta Crop Protection), Zampro® Fungicide (dimethomorph sold by BASF Corporation), Reason® 500 Fungicide (fenamidone sold by Bayer Crop Science) and Revus® Fungicide (mandipropamid sold by Syngenta Crop Protection) were sprayed as the synthetic benchmark comparisons.

TABLE 3a

| Sample Name | “0” | “0.25” | “0.5” | “1.0” | “Synth 1” | “Synth 2” | “Synth 3” | “Synth 4” |
|---|---|---|---|---|---|---|---|---|
| Serifel ® | 1.50 g | 1.50 g | 1.50 g | 1.50 g | — | — | — | — |
| Biofungicide | | | | | | | | |
| Additive Blend 6 | — | 2.50 g | 5.00 g | 10.0 g | — | — | — | — |
| Ridomil ® Gold | — | — | — | — | 3.0 g | — | — | — |
| Copper Fungicide | | | | | | | | |
| Quadris ® Fungicide | — | — | — | — | 1.8 g | | | — |
| Actigard ® Fungicide | — | — | — | — | — | 0.11 g | 0.11 g | 0.11 g |
| Orondis ® Ultra | — | — | — | — | — | 0.93 g | — | — |
| Zampro ® Fungicide | — | — | — | — | | — | 2.43 g | |
| Reason ® 500 | — | — | — | — | — | — | — | 0.97 g |
| Revus ® Fungicide | — | — | — | — | — | — | — | 1.37 g |
| Water (grams) | 999.62 | 997.23 | 994.84 | 990.06 | 995.20 | 998.96 | 997.46 | 998.63 |

Six applications were made over seven-day intervals to the same plots of spinach. At the time of spinach emergence, “Synth-1” was sprayed on all “Synth” plots. On weeks one and four “Synth2” was applied to “Synth” plots, on weeks 2 and 5 “Synth-3” was applied and on weeks 3 and 6 “Synth-4” was applied. At the end of the trial, the infection rates of Downy Mildew were evaluated by comparing to untreated spinach plots. Table 3b demonstrates the improved control of Downy Mildew (lower %-Infection) when Additive Blend 6 was added to Serifel® Biofungicide spray solutions as well as the relative rate effects.

TABLE 3b

| Sample | Untreated | “0” | “0.25” | “0.5” | “1.0” | “Synth” |
|---|---|---|---|---|---|---|
| %-Infection | 0.177 | 0.141 | 0.044 | 0.175 | 0.125 | 0.010 |
| Downy Mildew | | | | | | |
| Std Dev | 0.125 | 0.171 | 0.025 | 0.241 | 0.071 | 0.014 |
| Number of | 0 | 8.25E10 | 8.25E10 | 8.25E10 | 8.25E10 | 0 |
| CFU applied | | | | | | |
| Ratio | 0:0 | 1:0 | 1:1.67 | 1:3.33 | 1:6.67 | 0:0 |
| Serifel:Additive | | | | | | |

### Example 4

Eight solutions were prepared as described in Table 4a. These solutions were sprayed on sweet corn showing no signs of Northern Corn Leaf Blight (Setosphaeria turcicum). The bio-fungicide in the trial was Serifel® Biofungicide (Bacillus amyloliquefaciens sold by BASF Corporation). Six of the solutions included Additive Blend 3 (a 50:25:25 blend of POE 10 Soybean Oil, Di-isobutyl Naphthalene Sulfonate-Sodium salt and water). Headline® Fungicide (pyraclostrobin sold by BASF Corporation) was sprayed as the synthetic benchmark comparison.

TABLE 4a

| Sample Name | “0” | “0.25” | “0.5” | “1.0” | Headline | “A” | “B” | “C” |
|---|---|---|---|---|---|---|---|---|
| Serifel ® | 0.38 g | 0.38 g | 0.38 g | 0.38 g | — | — | — | — |
| Biofungicide | | | | | | | | |
| Additive Blend 3 | — | 2.39 g | 4.78 g | 9.56 g | — | 2.39 g | 4.78 g | 9.56 g |
| Headline ® | — | — | — | — | 4.93 g | — | — | — |
| Fungicide | | | | | | | | |
| Water (grams) | 999.62 | 997.23 | 994.84 | 990.06 | 999.46 | 997.61 | 995.22 | 990.44 |

Four applications were made over a month to the same sweet corn plots. At the end of the month, the infection rates of Northern Corn Leaf Blight were evaluated by comparing to untreated corn plots. The following table demonstrates the improved control of Northern Corn Leaf Blight (lower %-Infection) when Additive Blend 3 was added to Serifel® Biofungicide spray solutions as well as the relative rate effects.

TABLE 4b

| Sample | Untreated | “0” | “0.25” | “0.5” | “1.0” | “Headline” | “A” | “B” | “C” |
|---|---|---|---|---|---|---|---|---|---|
| %-Infection | 6.43 | 5.54 | 3.4 | 3.2 | 3.0 | 1.5 | 5.2 | 4.2 | 2.6 |
| Northern Corn | | | | | | | | | |
| Leaf Blight | | | | | | | | | |
| Std Dev | 0.83 | 1.30 | 0.55 | 0.45 | 0 | 0.85 | 0.45 | 0.45 | 0.89 |
| Number of | 0 | 2.0E10 | 2.0E10 | 2.0E10 | 2.0E10 | 0 | 0 | 0 | 0 |
| CFU applied | | | | | | | | | |
| Ratio | 0:0 | 1:0 | 1:6.37 | 1:12.75 | 1:25.5 | 0:0 | 0:6.37 | 0:12.75 | 0:25.5 |
| Serifel:Additive | | | | | | | | | |

### Example 5

Eight solutions were prepared as described in Table 5a. These solutions were sprayed on pumpkin plants showing no signs of Powdery Mildew (Podosphaera xanthii). The bio-fungicide in the trial was Serifel® Biofungicide (Bacillus amyloliquefaciens sold by BASF Corporation). Six of the solutions included methyl soyate. Pristine® Fungicide (pyraclostrobin and boscalid sold by BASF Corporation) was sprayed as the synthetic benchmark comparison.

TABLE 5a

| Sample Name | “0” | “0.25” | “0.5” | “1.0” | Pristine | “A” | “B” | “C” |
|---|---|---|---|---|---|---|---|---|
| Serifel ® | 0.38 g | 0.38 g | 0.38 g | 0.38 g | — | — | — | — |
| Biofungicide | | | | | | | | |
| Methyl Soyate | — | 2.39 g | 4.78 g | 9.56 g | — | 2.39 g | 4.78 g | 9.56 g |
| Pristine ® | — | — | — | — | 14.8 g | — | — | — |
| Fungicide | | | | | | | | |
| Water (grams) | 999.62 | 997.23 | 994.84 | 990.06 | 985.2 | 997.61 | 995.22 | 990.44 |

Four applications were made over a month to the same pumpkin plants. At the end of the month, the infection rates of Powdery Mildew were evaluated by comparing to untreated pumpkin plants. Table 5b demonstrates the improved control of Powdery Mildew (lower %-Infection) when methyl soyate was added to Serifel® Biofungicide spray solutions as well as the relative rate effects

TABLE 5b

| Sample | Untreated | “0” | “0.25” | “0.5” | “1.0” | “Pristine” | “A” | “B” | “C” |
|---|---|---|---|---|---|---|---|---|---|
| %-Infection | 21.8 | 17.8 | 15 | 12.5 | 2.5 | 3.8 | 10.0 | 9.0 | 9.5 |
| Powdery Mildew | | | | | | | | | |
| Std Dev | 3.66 | 3.14 | 0 | 2.89 | 0.58 | 1.25 | 0 | 1.15 | 1.00 |
| Number of | 0 | 2.0E10 | 2.0E10 | 2.0E10 | 2.0E10 | 0 | 0 | 0 | 0 |
| CFU applied | | | | | | | | | |
| Ratio | 0:0 | 1:0 | 1:6.37 | 1:12.75 | 1:25.5 | 0:0 | 0:6.37 | 0:12.75 | 0:25.5 |
| Serifel:Additive | | | | | | | | | |

### Example 6

Eight solutions were prepared as described in Table 6a. These solutions were sprayed on first year grape vines showing no signs of Downy Mildew (Plasmopara viticola). The bio-fungicide in the trial was Serifel® Biofungicide (Bacillus amyloliquefaciens sold by BASF Corporation). Six of the solutions included PEG 400 dioleate. Forum® Fungicide (dimethomorph sold by BASF Corporation) was sprayed as the synthetic benchmark comparison.

TABLE 6a

| Sample Name | “0” | “0.25” | “0.5” | “1.0” | Forum | “A” | “B” | “C” |
|---|---|---|---|---|---|---|---|---|
| Serifel ® | 0.38 g | 0.38 g | 0.38 g | 0.38 g | — | — | — | — |
| Biofungicide | | | | | | | | |
| PEG 400 dioleate | — | 2.39 g | 4.78 g | 9.56 g | — | 2.39 g | 4.78 g | 9.56 g |
| Forum ® Fungicide | — | — | — | — | 0.54 g | — | — | — |
| Water (grams) | 999.62 | 997.23 | 994.84 | 990.06 | 985.2 | 997.61 | 995.22 | 990.44 |

Four applications were made over a month to the same grape vines. At the end of the month, the infection rates of Downy Mildew were evaluated by comparing to untreated grape vines. Table 6b demonstrates the improved control of Downy Mildew (lower %-Infection) when PEG 400 dioleate was added to Serifel® Biofungicide spray solutions as well as the relative rate effects.

TABLE 6b

| Sample | Untreated | “0” | “0.25” | “0.5” | “1.0” | “Forum” | “A” | “B” | “C” |
|---|---|---|---|---|---|---|---|---|---|
| %-Infection | 12.2 | 8.3 | 2.0 | 2.2 | 0 | 1.6 | 2.5 | 2.5 | 2.0 |
| Downy Mildew | | | | | | | | | |
| Std Dev | 7.4 | 3.56 | 1.41 | 1.50 | 0 | 1.25 | 2.89 | 2.89 | 2.45 |
| Number of | 0 | 2.0E10 | 2.0E10 | 2.0E10 | 2.0E10 | 0 | 0 | 0 | 0 |
| CFU applied | | | | | | | | | |
| Ratio | 0:0 | 1:0 | 1:6.37 | 1:12.75 | 1:25.5 | 0:0 | 0:6.37 | 0:12.75 | 0:25.5 |
| Serifel:Additive | | | | | | | | | |

### Example 7

Five solutions were prepared as described in Table 7a. These solutions were sprayed on flowering strawberry plants showing no signs of Gray Mold (Botrytis cinerea). The bio-fungicide in the trial was Serifel® Biofungicide (Bacillus amyloliquefaciens sold by BASF Corporation). Three of the solutions included POE 10 Soybean Oil. Elevate® Fungicide (fenhexamid sold by BASF Corporation) was sprayed as the synthetic benchmark comparison.

TABLE 7a

| Sample Name | “0” | “0.25” | “0.5” | “1.0” | Elevate |
|---|---|---|---|---|---|
| Serifel ® Biofungicide | 0.38 g | 0.38 g | 0.38 g | 0.38 g | — |
| POE 10 Soybean Oil | — | 2.39 g | 4.78 g | 9.56 g | — |
| Elevate ® Fungicide | — | — | — | — | 1.80 g |
| Water (grams) | 999.62 | 997.23 | 994.84 | 990.06 | 998.2 |

Six applications were made at 7-day intervals to the same strawberry plants. After two weeks the plants were harvested of all berries. The berries were stored at room temperature for 3 days and then evaluated for Gray Mold. The same stored berries were evaluated again on day 5 of storage. At the end of each following week, berries were harvested, and the number of marketable berries evaluated at 3 days and 5 days after harvest. Table 7b demonstrates the improved control of Gray Mold (Higher %-Marketable Berries) when POE 10 Soybean Oil was added to Serifel® Biofungicide spray solutions as well as the relative rate effects.

TABLE 7b

| Sample | Untreated | “0” | “0.25” | “0.5” | “1.0” | “Elevate” |
|---|---|---|---|---|---|---|
| %-Marketable | 91.0 | 96.2 | 97.6 | 96.3 | 99.7 | 98.6 |
| @3 days | | | | | | |
| Std Dev | 9.4 | 4.9 | 4.1 | 2.9 | 0.55 | 1.4 |
| %-Marketable | 81.0 | 87.0 | 96.1 | 90.2 | 98.6 | 82.0 |
| @5 days | | | | | | |
| Std Dev | 17.3 | 13.8 | 5.0 | 8.3 | 2.2 | 22.5 |
| Number of | 0 | 2.0E10 | 2.0E10 | 2.0E10 | 2.0E10 | 0 |
| CFU applied | | | | | | |
| Ratio | 0:0 | 1:0 | 1:6.37 | 1:12.75 | 1:25.5 | 0:0 |
| Serifel:Additive | | | | | | |

### Example 8

Five solutions were prepared as described in Table 8a. These solutions were sprayed on flowering strawberry plants showing no signs of Gray Mold (Botrytis cinerea). The bio-fungicide in the trial was Serifel® Biofungicide (Bacillus amyloliquefaciens sold by BASF Corporation). Three of the solutions included PEG 400 diolate. Elevate® Fungicide (fenhexamid sold by BASF Corporation) was sprayed as the synthetic benchmark comparison.

TABLE 8a

| Sample Name | “0” | “0.25” | “0.5” | “1.0” | Elevate |
|---|---|---|---|---|---|
| Serifel ® Biofungicide | 0.38 g | 0.38 g | 0.38 g | 0.38 g | — |
| PEG 400 dioleate | — | 2.39 g | 4.78 g | 9.56 g | — |
| Elevate ® Fungicide | — | — | — | — | 1.80 g |
| Water (grams) | 999.62 | 997.23 | 994.84 | 990.06 | 998.2 |

Six applications were made at 7-day intervals to the same strawberry plants. After two weeks the plants were harvested of all berries. The berries were stored at room temperature for 3 days and then evaluated for Gray Mold. The same stored berries were evaluated again on day 5 of storage. At the end of each following week, berries were harvested, and the number of marketable berries evaluated at 3 days and 5 days after harvest. Table 8b demonstrates the improved control of Gray Mold (Higher %-Marketable Berries) when PEG 400 dioleate was added to Serifel® Biofungicide spray solutions as well as the relative rate effects.

TABLE 8b

| Sample | Untreated | “0” | “0.25” | “0.5” | “1.0” | “Elevate” |
|---|---|---|---|---|---|---|
| %-Marketable | 91.0 | 96.2 | 98.5 | 97.6 | 93.6 | 98.6 |
| @3 days | | | | | | |
| Std Dev | 9.4 | 4.9 | 1.3 | 4.0 | 12.4 | 1.4 |
| %-Marketable | 81.0 | 87.0 | 89.9 | 86.4 | 91.1 | 82.0 |
| @5 days | | | | | | |
| Std Dev | 17.3 | 13.8 | 8.7 | 18.7 | 10.8 | 22.5 |
| Number of | 0 | 2.0E10 | 2.0E10 | 2.0E10 | 2.0E10 | 0 |
| CFU applied | | | | | | |
| Ratio | 0:0 | 1:0 | 1:6.37 | 1:12.75 | 1:25.5 | 0:0 |
| Serifel:Additive | | | | | | |

### Example 9

Eight solutions were prepared as described in Table 9a. These solutions were sprayed on tomato plants showing no signs of Bacterial Speck (Pseudomonas syringae pv). The bio-fungicide in the trial was Serifel® Biofungicide (Bacillus amyloliquefaciens sold by BASF Corporation). Six of the solutions included Additive Blend 5 (a 25:7.5:16:20:7.5:24 blend of POE 10 Soybean Oil, PEG 400 dioleate, Di-isobutyl Naphthalene Sulfonate Sodium, an alkoxylated alcohol having an HLB value of 12, EO/PO/EO block co-polymer [molecular weight 2750 and HLB 2] and water). Kocide® 2000 Fungicide (copper hydroxide sold by Certis USA LLC) was sprayed as the synthetic benchmark comparison.

TABLE 9a

| Sample Name | “0” | “0.25” | “0.5” | “1.0” | Kocide | “A” | “B” | “C” |
|---|---|---|---|---|---|---|---|---|
| Serifel ® | 0.38 g | 0.38 g | 0.38 g | 0.38 g | — | — | — | — |
| Biofungicide | | | | | | | | |
| Additive Blend 5 | — | 2.39 g | 4.78 g | 9.56 g | — | 2.39 g | 4.78 g | 9.56 g |
| Kocide ® Fungicide | — | — | — | — | 1.20 g | — | — | — |
| Water (grams) | 999.62 | 997.23 | 994.84 | 990.06 | 998.8 | 997.61 | 995.22 | 990.44 |

Four applications were made over a month to the same tomato plants, which had been inoculated with the pathogen. At the end of the month, the infection rates of Bacterial Speck were evaluated by comparing to untreated tomato plants. Table 9b demonstrates the improved control of Bacterial Speck (lower %-Infection) when Additive Blend 5 was added to Serifel® Biofungicide spray solutions as well as the relative rate effects.

TABLE 9b

| Sample | Untreated | “0” | “0.25” | “0.5” | “1.0” | “Kocide” | “A” | “B” | “C” |
|---|---|---|---|---|---|---|---|---|---|
| %-Infection | 29 | 26.9 | 20 | 15 | 11.7 | 5.2 | 23.3 | 21.7 | 16.7 |
| Bacterial Speck | | | | | | | | | |
| Std Dev | 15.1 | 8 | 10 | 5 | 2.9 | 4.4 | 5.8 | 10.4 | 11.5 |
| Number of | 0 | 2.0E10 | 2.0E10 | 2.0E10 | 2.0E10 | 0 | 0 | 0 | 0 |
| CFU applied | | | | | | | | | |
| Ratio | 0:0 | 1:0 | 1:6.37 | 1:12.75 | 1:25.5 | 0:0 | 0:6.37 | 0:12.75 | 0:25.5 |
| Serifel:Additive | | | | | | | | | |

### Example 10

Eight solutions were prepared as described in Table 10a. These solutions were sprayed on tomato plants showing no signs of Bacterial Speck (Pseudomonas syringae pv). The bio-fungicide in the trial was Serifel® Biofungicide (Bacillus amyloliquefaciens sold by BASF Corporation). Six of the solutions included a C13-C15 EO/PO alkoxylated alcohol (HLB 12). Kocide® 2000 Fungicide (copper hydroxide sold by Certis USA LLC) was sprayed as the synthetic benchmark comparison.

TABLE 10a

| Sample Name | “0” | “0.25” | “0.5” | “1.0” | Kocide | “A” | “B” | “C” |
|---|---|---|---|---|---|---|---|---|
| Serifel ® | 0.38 g | 0.38 g | 0.38 g | 0.38 g | — | — | — | — |
| Biofungicide | | | | | | | | |
| Alkoxylated alcohol | — | 2.39 g | 4.78 g | 9.56 g | — | 2.39 g | 4.78 g | 9.56 g |
| Kocide ® Fungicide | — | — | — | — | 1.20 g | — | — | — |
| Water (grams) | 999.62 | 997.23 | 994.84 | 990.06 | 998.8 | 997.61 | 995.22 | 990.44 |

Four applications were made over a month to the same tomato plants, which had been inoculated with the pathogen. At the end of the month, the infection rates of Bacterial Speck were evaluated by comparing to untreated tomato plants. Table 10b demonstrates the improved control of Bacterial Speck (lower %-Infection) when the C13-C15 EO/PO alkoxylated alcohol (HLB 12) was added to Serifel® Biofungicide spray solutions as well as the relative rate effects.

TABLE 10b

| Sample | Untreated | “0” | “0.25” | “0.5” | “1.0” | “Kocide” | “A” | “B” | “C” |
|---|---|---|---|---|---|---|---|---|---|
| %-Infection | 29 | 26.9 | 16.7 | 13.3 | 10 | 5.2 | 14.7 | 13.8 | 15.4 |
| Bacterial Speck | | | | | | | | | |
| Std Dev | 15.1 | 7.1 | 11 | 5.8 | 0 | 4.4 | 2.9 | 5.8 | 6.2 |
| Number of | 0 | 2.0E10 | 2.0E10 | 2.0E10 | 2.0E10 | 0 | 0 | 0 | 0 |
| CFU applied | | | | | | | | | |
| Ratio | 0:0 | 1:0 | 1:6.37 | 1:12.75 | 1:25.5 | 0:0 | 0:6.37 | 0:12.75 | 0:25.5 |
| Serifel:Additive | | | | | | | | | |

### Example 11

Eight solutions were prepared as described in Table 11a. These solutions were sprayed on flowering primrose plants showing no signs of Gray Mold (Botrytis cinerea). The bio-fungicide in the trial was Serifel® Biofungicide (Bacillus amyloliquefaciens sold by BASF Corporation). Six of the solutions included a C13-C15 EO/PO alkoxylated alcohol (HLB 12). Elevate® Fungicide (fenhexamid sold by BASF Corporation) was sprayed as the synthetic benchmark comparison.

TABLE 11a

| Sample Name | “0” | “0.25” | “0.5” | “1.0” | Elevate | “A” | “B” | “C” |
|---|---|---|---|---|---|---|---|---|
| Serifel ® Biofungicide | 0.38 g | 0.38 g | 0.38 g | 0.38 g | — | — | — | — |
| Alkoxylated alcohol | — | 2.39 g | 4.78 g | 9.56 g | — | 2.39 g | 4.78 g | 9.56 g |
| Elevate ® Fungicide | — | — | — | — | 1.80 g | — | — | — |
| Water (grams) | 999.62 | 997.23 | 994.84 | 990.06 | 998.2 | 997.61 | 995.22 | 990.44 |

At the end of the month, the infection rates of Gray Mold were evaluated by comparing to untreated primrose plants. Table 11b demonstrates the improved control of Gray Mold (lower %-Infection) when the C13-C15 EO/PO alkoxylated alcohol (HLB 12) was added to Serifel® Biofungicide spray solutions as well as the relative rate effects.

TABLE 11b

| Sample | Untreated | “0” | “0.25” | “0.5” | “1.0” | “Elevate” | “A” | “B” | “C” |
|---|---|---|---|---|---|---|---|---|---|
| %-Infection | 35 | 29 | 24.2 | 20.8 | 14.2 | 6.7 | 23.3 | 21.7 | 34.2 |
| Gray Mold | | | | | | | | | |
| Std Dev | 2.5 | 2.7 | 1.8 | 1.6 | 0.9 | 0.7 | 2.8 | 1.8 | 1 |
| Number of | 0 | 2.0E10 | 2.0E10 | 2.0E10 | 2.0E10 | 0 | 0 | 0 | 0 |
| CFU applied | | | | | | | | | |
| Ratio | 0:0 | 1:0 | 1:6.37 | 1:12.75 | 1:25.5 | 0:0 | 0:6.37 | 0:12.75 | 0:25.5 |
| Serifel:Additive | | | | | | | | | |

### Example 12

Eight solutions were prepared as described in Table 12a. These solutions were sprayed on tomato plants showing heavy infections of Late Blight (Phytophthora infestans). The bio-fungicide in the trial was Serifel® Biofungicide (Bacillus amyloliquefaciens sold by BASF Corporation). Six of the solutions included an EO/PO/EO block co-polymer (molecular weight 2750 and HLB 2). Forum® Fungicide (dimethomorph sold by BASF Corporation) was sprayed as the synthetic benchmark comparison.

TABLE 12a

| Sample Name | “0” | “0.25” | “0.5” | “1.0” | Forum | “A” | “B” | “C” |
|---|---|---|---|---|---|---|---|---|
| Serifel ® Biofungicide | 0.38 g | 0.38 g | 0.38 g | 0.38 g | — | — | — | — |
| EO/PO/EO Block Polymer | — | 2.39 g | 4.78 g | 9.56 g | — | 2.39 g | 4.78 g | 9.56 g |
| Forum ® Fungicide | — | — | — | — | 0.54 g | — | — | — |
| Water (grams) | 999.62 | 997.23 | 994.84 | 990.06 | 985.2 | 997.61 | 995.22 | 990.44 |

Four applications were made over a month to the same tomato plants. At the end of the month, the infection rates of Late Blight were evaluated by comparing to untreated tomato plants. Table 12b demonstrates the improved control of Late Blight (lower %-Infection) when EO/PO/EO block co-polymer (molecular weight 2750 and HLB 2) was added to Serifel® Biofungicide spray solutions as well as the relative rate effects.

TABLE 12b

| Sample | Untreated | “0” | “0.25” | “0.5” | “1.0” | “Forum” | “A” | “B” | “C” |
|---|---|---|---|---|---|---|---|---|---|
| %-Infection | 75.2 | 62.8 | 20 | 13.3 | 16.7 | 9.5 | 23.3 | 18.3 | 30.3 |
| Late Blight | | | | | | | | | |
| Std Dev | 21.6 | 22.4 | 10 | 5.8 | 5.8 | 6.4 | 15.3 | 7.6 | 11.5 |
| Number of | 0 | 2.0E10 | 2.0E10 | 2.0E10 | 2.0E10 | 0 | 0 | 0 | 0 |
| CFU applied | | | | | | | | | |
| Ratio | 0:0 | 1:0 | 1:6.37 | 1:12.75 | 1:25.5 | 0:0 | 0:6.37 | 0:12.75 | 0:25.5 |
| Serifel:Additive | | | | | | | | | |

### Example 13

Eleven solutions were prepared as described in Table 13a. These solutions were applied to cucumber seed pots (8 pots per treatment) before germination. The pots had been filled with Pythium inoculated soil and 10 fresh cucumber seeds. The bio-fungicide in the trial was BlightBan® A506 Biofungicide (Pseudomonas fluorescens sold by NuFarm Americas Inc.). Ten of the solutions included a C6-C10 Fatty acid methyl ester.

TABLE 13a

| Sample Name | “0” | “0.1” | “0.25” | “1.0” | “2.5” | “10” |
|---|---|---|---|---|---|---|
| BlightBan ® A506 | 0.17 g | 0.17 g | 0.17 g | 0.17 g | 0.17 g | 0.17 g |
| C6-C10 Methyl Ester | — | 0.96 g | 2.39 g | 9.56 g | 23.9 g | 95.6 g |
| Water (grams) | 999.83 | 998.87 | 997.44 | 990.27 | 975.93 | 904.23 |

| Sample Name | “A” | “B” | “C” | “D” | “E” |
|---|---|---|---|---|---|
| BlightBan ® A506 | — | — | — | — | — |
| C6-C10 Methyl Ester | 0.96 g | 2.39 g | 9.56 g | 23.9 g | 95.6 g |
| Water (grams) | 999.04 | 997.61 | 990.44 | 976.10 | 904.4 |

After the solutions were poured on the pots, each pot was bottom watered and allowed to stand until 28 days. The number of seedlings emerging from the soil were counted and the vigor of each seedling rated as for biomass and root development. These pots were evaluated by comparing to untreated pots as well as pots of cucumber seed in sterilized soil. Table 13b demonstrates the improved control of Pythium (higher emergence) when C6-C10 Fatty acid methyl ester was added to BlightBan® A506 Biofungicide treatment solutions as well as the relative rate effects.

TABLE 13b

| Sample | Untreated | “0” | “0.1” | “0.25” | “1.0” | “2.5” | “10” |
|---|---|---|---|---|---|---|---|
| Cucumber Seeds | 0.77 | 1.88 | 2.88 | 2.86 | 3.00 | 1.50 | 0.25 |
| Germinated | | | | | | | |
| Number of | 0 | 1.7E9 | 1.7E9 | 1.7E9 | 1.7E9 | 1.7E9 | 1.7E9 |
| CFU applied | | | | | | | |
| Ratio Blight- | 0:0 | 1:0 | 1:5.62 | 1:14.1 | 1:56.2 | 1:140.6 | 1:562.4 |
| Ban:Additive | | | | | | | |

| Sample | Sterilized | “A” | “B” | “C” | “D” | “E” |
|---|---|---|---|---|---|---|
| Cucumber Seed | 2.0 | 1.88 | 3.13 | 0.75 | 0.88 | 0.5 |
| Germinated | | | | | | |
| Number of | 0 | 0 | 0 | 0 | 0 | 0 |
| CFU applied | | | | | | |
| Ratio | 0:0 | 0:5.62 | 0:14.1 | 0:56.2 | 0:140.6 | 0:562.4 |
| BlightBan:Additive | | | | | | |

### Example 14

Eleven solutions were prepared as described in Table 14a. These solutions were applied to cucumber seed pots (8 pots per treatment) before germination. The pots had been filled with Pythium inoculated soil and 10 fresh cucumber seeds. The bio-fungicide in the trial was BlightBan® A506 Biofungicide (Pseudomonas fluorescens sold by NuFarm Americas Inc.). Ten of the solutions included Soya fatty acid methyl ester.

TABLE 14a

| Sample Name | “0” | “0.1” | “0.25” | “1.0” | “2.5” | “10” |
|---|---|---|---|---|---|---|
| BlightBan ® A506 | 0.52 g | 0.52 g | 0.52 g | 0.52 g | 0.52 g | 0.52 g |
| Soya Methyl Ester | — | 0.96 g | 2.39 g | 9.56 g | 23.9 g | 95.6 g |
| Water (grams) | 999.48 | 998.52 | 997.09 | 989.92 | 975.58 | 903.88 |

| Sample Name | “A” | “B” | “C” | “D” | “E” |
|---|---|---|---|---|---|
| BlightBan ® A506 | — | — | — | — | — |
| Soya Methyl Ester | 0.96 g | 2.39 g | 9.56 g | 23.9 g | 95.6 g |
| Water (grams) | 999.04 | 997.61 | 990.44 | 976.10 | 904.4 |

After the solutions were poured on the pots, each pot was bottom watered and allowed to stand until 28 days. The number of seedlings emerging from the soil were counted and the vigor of each seedling rated as for biomass and root development. These pots were evaluated by comparing to untreated pots as well as pots of cucumber seed in sterilized soil. Table 14b demonstrates the improved control of Pythium (higher emergence) when Soya Fatty acid methyl ester was added to BlightBan® A506 Biofungicide treatment solutions as well as the relative rate effects.

TABLE 14b

| Sample | Untreated | “0” | “0.1” | “0.25” | “1.0” | “2.5” | “10” |
|---|---|---|---|---|---|---|---|
| Cucumber Seed | 0.77 | 1.88 | 2.63 | 3.13 | 3.25 | 1.63 | 0 |
| Germinated | | | | | | | |
| Number of | 0 | 5.2E9 | 5.2E9 | 5.2E9 | 5.2E9 | 5.2E9 | 5.2E9 |
| CFU applied | | | | | | | |
| Ratio | 0:0 | 1:0 | 1:1.85 | 1:4.60 | 1:18.5 | 1:46.0 | 1:184.6 |
| BlightBan:Additive | | | | | | | |

| Sample | Sterilized | “A” | “B” | “C” | “D” | “E” |
|---|---|---|---|---|---|---|
| Cucumber Seed | 2.0 | 1.38 | 1.25 | 0.25 | 1.00 | 0.38 |
| Germinated | | | | | | |
| Number of | 0 | 0 | 0 | 0 | 0 | 0 |
| CFU applied | | | | | | |
| Ratio | 0:0 | 0:1.85 | 0:4.6 | 0:18.5 | 0:46.0 | 0:184.6 |
| BlightBan:Additive | | | | | | |

### Example 15

Eleven solutions were prepared as described in Table 15a. These solutions were applied to cucumber seed pots (8 pots per treatment) before germination. The pots had been filled with Pythium inoculated soil and 10 fresh cucumber seeds. The bio-fungicide in the trial was BlightBan® A506 Biofungicide (Pseudomonas fluorescens sold by NuFarm Americas Inc.). Ten of the solutions included Oleic acid methyl ester.

TABLE 15a

| Sample Name | “0” | “0.1” | “0.25” | “1.0” | “2.5” | “10” |
|---|---|---|---|---|---|---|
| BlightBan ® A506 | 0.17 g | 0.17 g | 0.17 g | 0.17 g | 0.17 g | 0.17 g |
| Oleic Acid Methyl Ester | — | 0.96 g | 2.39 g | 9.56 g | 23.9 g | 95.6 g |
| Water (grams) | 999.83 | 998.87 | 997.44 | 990.27 | 975.93 | 904.23 |

| Sample Name | “A” | “B” | “C” | “D” | “E” |
|---|---|---|---|---|---|
| BlightBan ® A506 | — | — | — | — | — |
| Oleic Acid Methyl Ester | 0.96 g | 2.39 g | 9.56 g | 23.9 g | 95.6 g |
| Water (grams) | 999.04 | 997.61 | 990.44 | 976.10 | 904.4 |

After the solutions were poured on the pots, each pot was bottom watered and allowed to stand until 28 days. The number of seedlings emerging from the soil were counted and the vigor of each seedling rated as for biomass and root development. These pots were evaluated by comparing to untreated pots as well as pots of cucumber seed in sterilized soil. Table 15b demonstrates the improved control of Pythium (higher emergence) when Oleic acid methyl ester was added to BlightBan® A506 Biofungicide treatment solutions as well as the relative rate effects.

TABLE 15b

| Sample | Untreated | “0” | “0.1” | “0.25” | “1.0” | “2.5” | “10” |
|---|---|---|---|---|---|---|---|
| Cucumber Seed | 0.77 | 1.75 | 2.50 | 2.50 | 2.75 | 1.88 | 0 |
| Germinated | | | | | | | |
| Number of | 0 | 1.7E9 | 1.7E9 | 1.7E9 | 1.7E9 | 1.7E9 | 1.7E9 |
| CFU applied | | | | | | | |
| Ratio | 0:0 | 1:0 | 1:5.62 | 1:14.1 | 1:56.2 | 1:140.6 | 1:562.4 |
| BlightBan:Additive | | | | | | | |

| Sample | Sterilized | “A” | “B” | “C” | “D” | “E” |
|---|---|---|---|---|---|---|
| Cucumber Seed | 2.0 | 1.38 | 1.38 | 2.13 | 1.25 | 0.38 |
| Germinated | | | | | | |
| Number of | 0 | 0 | 0 | 0 | 0 | 0 |
| CFU applied | | | | | | |
| Ratio | 0:0 | 0:5.62 | 0:14.1 | 0:56.2 | 0:140.6 | 0:562.4 |
| BlightBan:Additive | | | | | | |

### Example 16

Eleven solutions were prepared as described in Table 16a. These solutions were applied to cucumber seed pots (8 pots per treatment) before germination. The pots had been filled with Pythium inoculated soil and 10 fresh cucumber seeds. The bio-fungicide in the trial was BlightBan® A506 Biofungicide (Pseudomonas fluorescens sold by NuFarm Americas Inc.). Ten of the solutions included Oleic acid methyl ester.

TABLE 16a

| Sample Name | “0” | “0.1” | “0.25” | “1.0” | “2.5” | “10” |
|---|---|---|---|---|---|---|
| BlightBan ® A506 | 0.52 g | 0.52 g | 0.52 g | 0.52 g | 0.52 g | 0.52 g |
| Oleic Acid Methyl Ester | — | 0.96 g | 2.39 g | 9.56 g | 23.9 g | 95.6 g |
| Water (grams) | 999.48 | 998.52 | 997.09 | 989.92 | 975.58 | 903.88 |

| Sample Name | “A” | “B” | “C” | “D” | “E” |
|---|---|---|---|---|---|
| BlightBan ® A506 | — | — | — | — | — |
| Oleic Acid Methyl Ester | 0.96 g | 2.39 g | 9.56 g | 23.9 g | 95.6 g |
| Water (grams) | 999.04 | 997.61 | 990.44 | 976.10 | 904.4 |

After the solutions were poured on the pots, each pot was bottom watered and allowed to stand until 28 days. The number of seedlings emerging from the soil were counted and the vigor of each seedling rated as for biomass and root development. These pots were evaluated by comparing to untreated pots as well as pots of cucumber seed in sterilized soil. Table 16b demonstrates the improved control of Pythium (higher emergence) when Oleic acid methyl ester was added to BlightBan® A506 Biofungicide treatment solutions as well as the relative rate effects.

TABLE 16b

| Sample | Untreated | “0” | “0.1” | “0.25” | “1.0” | “2.5” | “10” |
|---|---|---|---|---|---|---|---|
| Cucumber Seed | 0.77 | 1.75 | 2.0 | 0.75 | 1.0 | 0.38 | 0.25 |
| Germinated | | | | | | | |
| Number of | 0 | 5.2E9 | 5.2E9 | 5.2E9 | 5.2E9 | 5.2E9 | 5.2E9 |
| CFU applied | | | | | | | |
| Ratio | 0:0 | 1:0 | 1:1.85 | 1:4.60 | 1:18.5 | 1:46.0 | 1:184.6 |
| BlightBan:Additive | | | | | | | |

| Sample | Sterilized | “A” | “B” | “C” | “D” | “E” |
|---|---|---|---|---|---|---|
| Cucumber Seed | 2.0 | 1.38 | 1.38 | 2.13 | 1.25 | 0.38 |
| Germinated | | | | | | |
| Number of | 0 | 0 | 0 | 0 | 0 | 0 |
| CFU applied | | | | | | |
| Ratio | 0:0 | 0:1.85 | 0:4.6 | 0:18.5 | 0:46.0 | 0:184.6 |
| BlightBan:Additive | | | | | | |

### Example 17

Eleven solutions were prepared as described in Table 17a. These solutions were applied to cucumber seed pots (8 pots per treatment) before germination. The pots had been filled with Pythium inoculated soil and 10 fresh cucumber seeds. The bio-fungicide in the trial was Subtilex® NG Biofungicide (Bacillus subtilis sold by BASF Corporation). Ten of the solutions included PEG 400 Dioleate.

TABLE 17a

| Sample Name | “0” | “0.1” | “0.25” | “1.0” | “2.5” | “10” |
|---|---|---|---|---|---|---|
| Subtilex ® NG | 0.30 g | 0.30 g | 0.30 g | 0.30 g | 0.30 g | 0.30 g |
| PEG 400 Dioleate | — | 0.96 g | 2.39 g | 9.56 g | 23.9 g | 95.6 g |
| Water (grams) | 999.70 | 998.74 | 997.31 | 990.14 | 975.80 | 904.10 |

| Sample Name | “A” | “B” | “C” | “D” | “E” |
|---|---|---|---|---|---|
| Subtilex ® NG | — | — | — | — | — |
| PEG 400 Dioleate | 0.96 g | 2.39 g | 9.56 g | 23.9 g | 95.6 g |
| Water (grams) | 999.04 | 997.61 | 990.44 | 976.10 | 904.4 |

After the solutions were poured on the pots, each pot was bottom watered and allowed to stand until 28 days. The number of seedlings emerging from the soil were counted and the vigor of each seedling rated as for biomass and root development. These pots were evaluated by comparing to untreated pots as well as pots of cucumber seed in sterilized soil. Table 17b demonstrates the improved control of Pythium (higher emergence) when PEG 400 Dioleate was added to Subtilex® NG Biofungicide treatment solutions as well as the relative rate effects.

TABLE 17b

| Sample | Untreated | “0” | “0.1” | “0.25” | “1.0” | “2.5” | “10” |
|---|---|---|---|---|---|---|---|
| Cucumber Seed | 0.875 | 0.375 | 1.00 | 1.125 | 1.375 | 1.50 | 0.875 |
| Germinated | | | | | | | |
| Number of | 0 | 1.65E10 | 1.65E10 | 1.65E10 | 1.65E10 | 1.65E10 | 1.65E10 |
| CFU applied | | | | | | | |
| Ratio | 0:0 | 1:0 | 1:3.2 | 1:8 | 1:32 | 1:80 | 1:320 |
| Subtilex:Additive | | | | | | | |

| Sample | Sterilized | “A” | “B” | “C” | “D” | “E” |
|---|---|---|---|---|---|---|
| Cucumber Seed | 1.125 | 1.125 | 1.5 | 1.25 | 0.75 | 0.5 |
| Germinated | | | | | | |
| Number of | 0 | 0 | 0 | 0 | 0 | 0 |
| CFU applied | | | | | | |
| Ratio | 0:0 | 0:3.2 | 0:8 | 0:32 | 0:80 | 0:320 |
| Subtilex:Additive | | | | | | |

1. A spraying liquid suitable for spraying plants, comprising:
a. 0.1%-v/v to 2%-v/v of at least one additive selected from the group consisting of i to x:
i. EO-PO block polymers having a HLB (hydrophilic-lipophilic-balance) value of 1, 2, 3, 4, 6, 8, 12, 13, 14, 15, 27 or 29 and an average molecular weight (AMW) between 1500 and 15000,
ii. methyl esters of unsaturated or saturated C6 to C18 fatty acids,
iii. ethylenediamine tetra-functional PO/EO block polymers with a molecular weight (AMW) between 2000 and 20000 s,
iv. C8 to C14 alkyl polyglycosides,
v. fatty acid polyethylene esters comprising C12 to C18 saturated or unsaturated fatty acids
vi. ethoxylated triglycerides derived from plant oils,
vii. alkoxylated alcohols, having from 2 to 80 oxyethylene units and from 2 to 40 oxypropylene units and C4 to C18 alcohols,
viii. alkyl naphthalene sulfonates,
ix. alkyl polyglycoside lignosulfonate blends, and
x. polyquaternium polymers,
b. 0.002%-v/v to 2.0%-v/v bacterial spores or vegetative cells,
c. optionally 0.001%-v/v to 10.0%-v/v of further components, and
d. up to 100%-v/v water.

2. The spraying liquid of claim 1, wherein at least one additive is selected from the group consisting of EO/PO/EO block polymers with an HLB value of 1, 2, 3, 12, 13, 14, 15, 27 or 29, PO/EO/PO block polymers with an HLB value of 4, 6, 8, 12 or 15, C6-C10 fatty acid methyl ester, C12-C18 fatty acid methyl ester, soya fatty acid methyl ester, oleic acid methyl ester, ethylenediamine tetrafunctional PO/EO block polymers with an molecular weight of 3600, 4700 or 15000, C8-C10 alkylpolyglycosides, PEG 400 monolaurate, PEG 400 monooleate, PEG 400 dioleate, soybean oil POE 10, castor oil POE 16, castor oil POE 40, alkoxylated alcohol HLB 9 or 12, butyl alcohol POP 31 POE 31, dibutyl naphthalene sulfonate, and a lignosulfonate C8-C10 alkylpolyglycoside blend.

3. The spraying liquid of claim 1, wherein the spraying liquid comprises a blend of additives comprising one or more additives selected from the group consisting of ethoxylated triglycerides and one or more additives selected from the group consisting of a) to d):
a) EO-PO block polymers having a HLB (hydrophilic-lipophilic-balance) value of 1, 2, 3, 4, 6, 8, 12, 13, 14, 15, 27 or 29 and an average molecular weight between 1500 and 15000,
b) fatty acid polyethylene esters,
c) alkoxylated alcohols, and
d) alkyl naphthalene sulfonates,
wherein the % v/v ratio of the additives of the two groups is between 10:1 and 1:1, and if one or more additives of the group of ethoxylated triglycerides and/or group a), b), c) or d) is present, the total amount of all additives per group is calculated to determine the ratio.

4. The spraying liquid of claim 1, wherein the spraying liquid comprises a blend of additives as defined in a to g:
a. an additive blend comprising 1 to 99% v/v of at least one additive selected from the group consisting of: soybean oil POE 10, castor oil POE 16 and castor oil POE 40, and 1 to 99% v/v of at least one additive selected from the group consisting of: PEG 400 monolaurate, PEG 400 monooleate and PEG 400 dioleate, wherein the % v/v of both components add up to 100%;
b. an additive blend comprising 1 to 98% v/v of at least one additive selected from the group consisting of: soybean oil POE 10, castor oil POE 16 and castor oil POE 40, and 1 to 98% v/v of at least one additive selected from the group consisting of: PEG 400 monolaurate, PEG 400 monooleate and PEG 400 dioleate;
c. an additive blend comprising 1 to 99% v/v of at least one additive selected from the group consisting of: soybean oil POE 10, castor oil POE 16 and castor oil POE 40, and 1 to 99% v/v of dibutyl naphthalene sulfonate, wherein the % v/v of all components add up to 100%;
d. an additive blend comprising 1 to 98% v/v of at least one additive selected from the group consisting of: soybean oil POE 10, castor oil POE 16 and castor oil POE 40 and 1 to 98% v/v of at least one additive selected from the group consisting of: PEG 400 monolaurate, PEG 400 monooleate and PEG 400 dioleate, and 1 to 98% v/v of at least one additive selected from the group consisting of: EO/PO/EO block polymers with an HLB value of 1, 2, 3, 12, 13, 14, 15, 27 and 29, wherein the % v/v of all components add up to 100%;
e. an additive blend comprising 1 to 95% v/v of at least one additive selected from the group consisting of: soybean oil POE 10, castor oil POE 16 and castor oil POE 40, and 1 to 95% v/v of at least one additive selected from the group consisting of: PEG 400 monolaurate, PEG 400 monooleate and PEG 400 dioleate and 1 to 95% v/v dibutyl naphthalene sulfonate, and 1 to 95% v/v of at least one additive selected from the group consisting of: alkoxylated alcohol having an HLB value of 9 and 12, and 1 to 95% v/v at least one selected from the group consisting of: EO/PO/EO block polymers with an HLB value of 1, 2, 3, 12, 13, 14, 15, 27 and 29, wherein the % v/v of all components add up to 100%;
f. an additive blend comprising 1 to 97% v/v of at least one additive selected from the group consisting of: soybean oil POE 10, castor oil POE 16, castor oil POE 40, 1% to 97% v/v dibutyl naphthalene sulfonate, and 1 to 97% v/v an alkoxylated alcohol having an HLB value of 9 or 12, wherein the % v/v of all components add up to 100%;
g. an additive blend comprising 1 to 97% v/v of at least one additive selected from the group consisting of: EO/PO/EO block polymers with an HLB value of 1, 2, 3, 12, 13, 14, 15, 27 and 29, and 1 to 97% v/v of at least one additive selected from the group consisting of: PEG 400 monolaurate, PEG 400 monooleate and PEG 400 dioleate, and 1 to 97% v/v of at least one additive selected from the group consisting of: soybean oil POE 10, castor oil POE 16 and castor oil POE 40 wherein the % v/v of all components add up to 100%.

5. The spraying liquid of claim 1, wherein the bacterial spores or vegetative cells are from the genus Bacillus, Lysinibacilllus, Paenibacillus or Streptomyces.

6. The spraying liquid of claim 1, wherein the bacterial spores or vegetative cells are from the genus Pseudomonas, Burkholderia, Paraburkholderia or Rhizobium.

7. An additive blend comprising one or more additives selected from the group consisting of ethoxylated triglycerides and one or more additives selected from the group consisting of A1) to D1):
A1) EO-PO block polymers having a HLB (hydrophilic-lipophilic-balance) value of 1, 2, 3, 4, 6, 8, 12, 13, 14, 15, 27 or 29 and an average molecular weight between 1500 and 15000,
B1) fatty acid polyethylene esters,
C1) alkoxylated alcohols, and
D1) alkyl naphthalene sulfonates,
wherein the % v/v ratio of the additives of the two groups is between 10:1 and 1:1, and if one or more additives of the group of ethoxylated triglycerides and/or group A1), B1), C1) or D1) is present, the total amount of all additives per group is calculated to determine the ratio,
or comprising one or more additives selected from the group consisting of EO-PO block polymers having a HLB (hydrophilic-lipophilic-balance) value of 1, 2, 3, 4, 6, 8, 12, 13, 14, 15, 27 and 29 and an average molecular weight between 1500 and 15000 and one or more additives selected from the group consisting of A2) to C2):
A2) fatty acid methyl esters;
B2) ethylenediamine tetra-functional PO/EO block polymers;
C2) fatty acid polyethylene esters,
wherein the % v/v ratio of the additives of the group of EO-PO block polymers and the one or more additives of the groups of A2), B2) or C2) is between 10:1 and 1:1, and if one or more additives of the group of EO-PO block polymers and/or group A2), B3), or C2) is present, the total amount of all additives per group is calculated to determine the ratio,
or comprising one or more additives selected from the group consisting of EO/PO/EO block polymers having a HLB (hydrophilic-lipophilic-balance) value of 1, 2, 3, 4, 6, 8, 12, 13, 14, 15, 27 or 29 and an average molecular weight between 1500 and 15000 and
one or more additives selected from the group consisting of PO/EO/PO block polymers with an HLB value of 4, 6, 8, or 12 and an average molecular weight between 1500 and 4000,
wherein the % v/v ratio of the EO/PO/EO block polymers and the PO/EO/PO block polymers is between 100:1 and 1:1, and if one or more additives of the group of EO/PO/EO block polymers and/or the group of PO/EO/PO block polymers is present, the total amount of all additives per group is calculated to determine the ratio.

8. An additive blend comprising a blend of additives as defined in any one of the alternatives a to g:
a. comprising 1 to 99% v/v of at least one additive selected from the group consisting of: soybean oil POE 10, castor oil POE 16 and castor oil POE 40, and 1 to 99% v/v of at least one additive selected from the group consisting of: PEG 400 monolaurate, PEG 400 monooleate and PEG 400 dioleate,
b. comprising 1 to 98% v/v of at least one additive selected from the group consisting of: soybean oil POE 10, castor oil POE 16 and castor oil POE 40, and 1 to 98% v/v of at least one additive selected from the group consisting of: PEG 400 monolaurate, PEG 400 monooleate and PEG 400 dioleate,
c. comprising 1 to 99% v/v of at least one additive selected from the group consisting of: soybean oil POE 10, castor oil POE 16, castor oil POE 40, and 1 to 99% v/v of dibutyl naphthalene sulfonate,
d. comprising 1 to 98% v/v of at least one additive selected from the group consisting of: soybean oil POE 10, castor oil POE 16 and castor oil POE 40, and 1 to 98% v/v of at least one additive selected from the group consisting of: PEG 400 monolaurate, PEG 400 monooleate and PEG 400 dioleate, and 1 to 98% v/v of at least one additive selected from the group consisting of: EO/PO/EO block polymers with an HLB value of 1, 2, 3, 12, 13, 14, 15, 27 and 29,
e. comprising 1 to 95% v/v of at least one additive selected from the group consisting of: soybean oil POE 10, castor oil POE 16 and castor oil POE 40, and 1 to 95% v/v of at least one additive selected from the group consisting of: PEG 400 monolaurate, PEG 400 monooleate, and PEG 400 dioleate, and 1 to 95% v/v dibutyl naphthalene sulfonate, and 1 to 95% v/v of at least one additive selected from the group consisting of: alkoxylated alcohol having an HLB value of 9 or 12, and 1 to 95% v/v at least one selected from the group consisting of: EO/PO/EO block polymers with an HLB value of 1, 2, 3, 12, 13, 14, 15, 27 and 29,
f. comprising 1 to 97% v/v of at least one additive selected from the group consisting of: soybean oil POE 10, castor oil POE 16, and castor oil POE 40, 1 to 97% v/v dibutyl naphthalene sulfonate, and 1 to 97% v/v an alkoxylated alcohol having an HLB value of 9 or 12,
g. comprising 1 to 97% v/v of at least one additive selected from the group consisting of: EO/PO/EO block polymers with an HLB value of 1, 2, 3, 12, 13, 14, 15, 27 and 29, and 1 to 97% v/v of at least one additive selected from the group consisting of: PEG 400 monolaurate, PEG 400 monooleate and PEG 400 dioleate, and 1 to 97% v/v of at least one additive selected from the group consisting of: soybean oil POE 10, castor oil POE 16 and castor oil POE 40,
wherein the % v/v of all components of additive blends a to g and optionally water add up to 100%.

9. A kit of at least two parts to prepare a spraying liquid suitable for spraying plants or for preparing a seed treatment composition for treating seeds of plants, wherein bacterial spores or vegetative cells are provided in a first concentrated form and at least one of the additive blends of claim 7 is provided in at least one further concentrated form and wherein the relative amounts of the bacterial spores or vegetative cells are 0.002%-v/v to 2.0%-v/v and the relative amounts of the additive are 0.1%-v/v to 2%-v/v.

10. A method to control phytopathogenic fungi or phytopathogenic bacteria wherein a kit of at least two parts as claimed in claim 9 is used to prepare a spraying liquid for spraying plants or for preparing a seed treatment composition for treating seeds and the spraying liquid for spraying plants is sprayed on plants or the plant seeds are treated with the seed treatment composition for treating seeds.

11. A method to control phytopathogenic fungi or phytopathogenic bacteria, the method comprising using the additive blend of claim 7, to enhance the pesticidal effectiveness of a pesticidal microorganism.

12. A method of using the additive blend of claim 7, the method comprising using the additive blend to prepare a spraying liquid.

13. A method of using the spraying liquid of claim 1, the method comprising using the spraying liquid to control phytopathogenic fungi or phytopathogenic bacteria.

14. A method of using the kit of at least two parts of claim 9, the method comprising using the kit to prepare a spraying liquid.

15. A kit of at least two parts to prepare a spraying liquid suitable for spraying plants or for preparing a seed treatment composition for treating seeds of plants, wherein bacterial spores or vegetative cells are provided in a first concentrated form and at least one of the additive blends of claim 8 is provided in at least one further concentrated form and wherein the relative amounts of the bacterial spores or vegetative cells are 0.002%-v/v to 2.0%-v/v and the relative amounts of the additive are 0.1%-v/v to 2%-v/v.

16. A method to control phytopathogenic fungi or phytopathogenic bacteria wherein a kit of at least two parts as claimed in claim 15 is used to prepare a spraying liquid for spraying plants or for preparing a seed treatment composition for treating seeds and the spraying liquid for spraying plants is sprayed on plants or the plant seeds are treated with the seed treatment composition for treating seeds.

17. A method to control phytopathogenic fungi or phytopathogenic bacteria, the method comprising using the additive blend of claim 8, to enhance the pesticidal effectiveness of a pesticidal microorganism.

18. A method of using the additive blend of claim 8, the method comprising using the additive blend to prepare a spraying liquid.

19. A method of using the kit of at least two parts of claim 15, the method comprising using the kit to prepare a spraying liquid.

20. The spraying liquid of claim 1, wherein the spraying liquid comprises a blend of additives comprising one or more additives selected from the group consisting of ethoxylated soybean oil with POE 10, 30, 42 or 60, ethoxylated castor oil with POE 2.5, 5, 7, 16, 18, 20, 25, 30, 33, 35, 36, 40, 44, 60 or 200, and ethoxylated rapeseed oil with POE 30 and one or more additives selected from the group consisting of a) to d):
a) EO-PO block polymers having a HLB (hydrophilic-lipophilic-balance) value of 1, 2, 3, 4, 6, 8, 12, 13, 14, 15, 27 or 29 and an average molecular weight between 1500 and 15000,
b) fatty acid polyethylene esters,
c) alkoxylated alcohols, and
d) alkyl naphthalene sulfonates,
wherein the % v/v ratio of the additives of the two groups is between 10:1 and 1:1, and if one or more additives of the group of ethoxylated triglycerides and/or group a), b), c) or d) is present, the total amount of all additives per group is calculated to determine the ratio.
